(19) United States
(12) Patent Application Publication
(10) Pub. No.: US 2016/0204352 A1
(43) Pub. Date: Jul. 14, 2016

(54) COMPOUND HAVING TRIPHENYLENE RING STRUCTURE, AND ORGANIC ELECTROLUMINESCENT DEVICE

(71) Applicants: HODOGAYA CHEMICAL CO., LTD., Tokyo (JP); KYUSHU UNIVERSITY, NATIONAL UNIVERSITY CORPORATION, Fukuoka-shi (JP)

(72) Inventors: Chihaya Adachi, Fukuoka-shi (JP); Takuma Yasuda, Fukuoka-shi (JP); Katsuyuki Shizu, Fukuoka-shi (JP); Takehiro Takahashi, Tokyo (JP); Kazunori Togashi, Tokyo (JP)

(21) Appl. No.: 14/914,403

(22) PCT Filed: Aug. 11, 2014
(86) PCT No.: PCT/JP2014/004172

§ 371 Date: Feb. 25, 2016

(30) Foreign Application Priority Data
Aug. 27, 2013 (JP) 2013-175995

(51) Int. Cl. H01L 51/00; C09K 11/06; C07D 209/88; C07D 265/38; C07D 219/02

(52) U.S. Cl. CPC H01L 51/0054; C07D 265/38; C07D 219/02; C07D 209/88; C09K 11/06; H01L 51/0071; H01L 51/0072; C09K 2211/1033; C09K 2211/1029; H01L 51/5012

(57) ABSTRACT

A compound that emits fluorescence and delayed fluorescence is provided as a material for an organic electroluminescent device of high efficiency, and an organic photoluminescent device and an organic electroluminescent device of high efficiency and high luminance are provided using this compound. The compound of a general formula (1) having a triphenylene ring structure is used as a constituent material of at least one organic layer in the organic electroluminescent device that includes a pair of electrodes, and one or more organic layers sandwiched between the pair of electrodes.

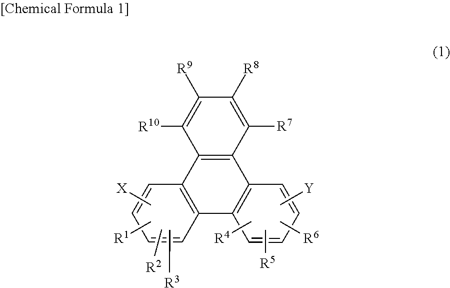

### TECHNICAL FIELD

The present invention relates to compounds suitable for an organic electroluminescent device which is a preferred self-luminous device for various display devices, and relates to the organic electroluminescent device. Specifically, this invention relates to compounds having a triphenylene ring structure, and organic electroluminescent devices using the compounds.

### BACKGROUND ART

The organic electroluminescent device is a self-luminous device and has been actively studied for their brighter, superior visibility and the ability to display clearer images in comparison with liquid crystal devices.

In an attempt to improve the device luminous efficiency, there have been developed devices that use phosphorescent materials to generate phosphorescence, specifically that make use of the emission from the triplet excitation state. According to the excitation state theory, phosphorescent materials are expected to greatly improve luminous efficiency as much as about four times that of the conventional fluorescence.

In 1993, M. A. Baldo et al. at Princeton University realized 8% external quantum efficiency with a phosphorescent device using an iridium complex.

Devices that use light emission caused by thermally activated delayed fluorescence (TADF) have also been developed. In 2011, Adachi et al. at Kyushu University, National University Corporation realized 5.3% external quantum efficiency with a device using a thermally activated delayed fluorescent material (refer to Non-Patent Document 1, for example).

In an organic electroluminescent device, carriers are injected from each of both electrodes, i.e., positive and negative electrodes to a light-emitting substance to generate a light-emitting substance in an excited state so as to emit light. It is generally said that in the case of a carrier injection type organic EL device, 25% of generated excitons are excited to an excited singlet state and the remaining 75% are excited to an excited triplet state. Accordingly, it is conceivable that utilization of light to be emitted from the excited triplet state, i.e., phosphorescence should provide higher energy use efficiency. However, in the phosphorescence, the excited triplet state has a long lifetime, and hence deactivation of energy occurs through saturation of an excited state and interactions with excitons in an excited triplet state, with the result that a high quantum yield is not obtained in many cases in general.

In view of the foregoing, an organic electroluminescent device utilizing a material which emits delayed fluorescence is conceivable. A certain kind of fluorescent substance emits fluorescence via intersystem crossing or the like leading to energy transition to an excited triplet state and the subsequent reverse intersystem crossing to an excited singlet state through triplet-triplet annihilation or thermal energy absorption. In the organic electroluminescent device, it is considered that the latter material which emits thermally activated delayed fluorescence is particularly useful. In this case, when a delayed fluorescent material is utilized in the organic electroluminescent device, excitons in an excited singlet state emit fluorescence as per normal. On the other hand, excitons in an excited triplet state absorb heat produced from a device and undergo intersystem crossing to an excited singlet to emit fluorescence. The fluorescence in this case is light emission from the excited singlet and hence is light emission at the same wavelength as fluorescence. However, the fluorescence has a longer lifetime of light to be emitted, i.e., a longer emission lifetime than those of normal fluorescence and phosphorescence by virtue of reverse intersystem crossing from an excited triplet state to an excited singlet state, and hence is observed as fluorescence delayed as compared to the normal fluorescence and phosphorescence. This can be defined as delayed fluorescence. Through the use of such thermally activated type exciton transfer mechanism, i.e., through thermal energy absorption after carrier injection, the ratio of a compound in an excited singlet state, which has usually been generated only at a ratio of 25%, can be increased to 25% or more. The use of a compound which emits intense fluorescence and delayed fluorescence even at a low temperature of less than 100° C. results in sufficient intersystem crossing from an excited triplet state to an excited singlet state by means of heat of a device, contributing to emission of delayed fluorescence. Thus, the luminous efficiency is drastically improved (refer to Patent Document 1 and Patent Document 2, for example).

Compounds of the following general formula (X) having a triphenylene structure are proposed as a light-emitting material and a hole transport material (refer to Patent Document 3, for example).

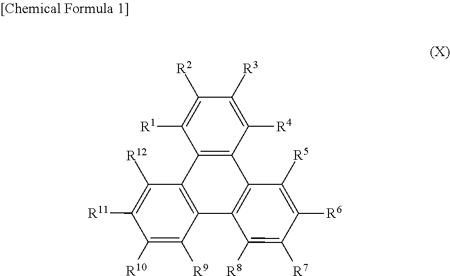

Although the above compounds are disclosed as a light-emitting material attaining light emission of the compounds themselves, emission of delayed fluorescence is neither disclosed nor suggested.

### CITATION LIST

### Patent Documents

### Patent Document 1: JP-A-2004-241374

### Patent Document 2: JP-A-2006-024830

### Patent Document 3: JP-A-11-251063

### Non-Patent Documents

Non-Patent Document 1: Appl. Phys. Let., 98, 083302 (2011)
Non-Patent Document 2: Synth. Commun., 11, 513 (1981)
Non-Patent Document 3: Appl. Phys. Let., 101, 093306 (2012)
Non-Patent Document 4: Chem. Commun., 48, 11392 (2012)

### Non-Patent Document 5: NATURE 492, 235 (2012)

Non-Patent Document 6: Organic EL Symposium, the 1st Regular presentation Preprints, 19 (2005)

### SUMMARY OF THE INVENTION

### Problems to be Solved by the Invention

An object of the present invention is to provide a compound that emits fluorescence and delayed fluorescence as a material for an organic electroluminescent device of high efficiency, and to provide an organic photoluminescent device (hereinafter referred to as an organic PL device) and an organic electroluminescent device of high efficiency and high luminance using this compound.

### Means for Solving the Problems

To achieve the above object, the present inventors have noted compounds having a triphenylene ring structure with heterocyclic structures such as a phenoxazine ring and a phenothiazine ring, and designed and chemically synthesized compounds using, as indexes, a difference between excited triplet energy and excited singlet energy (ΔE_{ST}), and oscillator strength (f) which are obtained by theoretical calculation. As a result of actually measuring the emission (PL) spectrums of the chemically synthesized compounds, the present inventors found new compounds having a triphenylene ring structure which emit delayed fluorescence. The present inventors produced various test organic electroluminescent devices using these compounds, and the present invention was completed after thorough evaluations of the device characteristics.

1) The present invention is a compound of the following general formula (1) having a triphenylene ring structure.

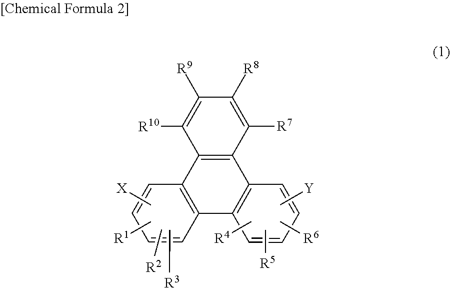

In the formula, X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group. Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group. R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

2) The present invention is the compound having a triphenylene ring structure according to 1), the compound being represented by the following general formula (1-1).

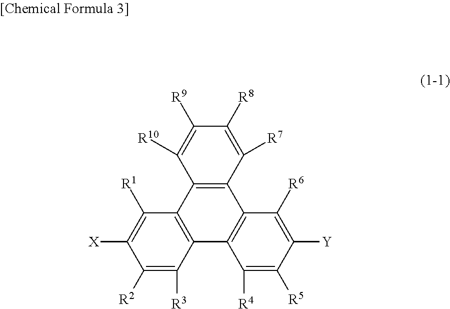

In the formula, X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group. Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group. R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

3) The present invention is the compound having a triphenylene ring structure according to 1), the compound being represented by the following general formula (1-2).

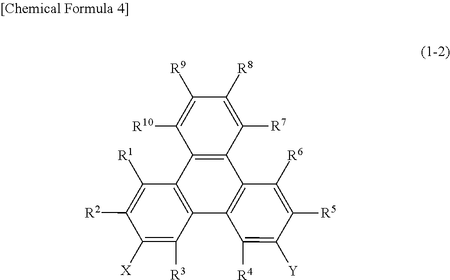

In the formula, X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group. Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group. R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

4) The present invention is the compound having a triphenylene ring structure according to 1), the compound being represented by the following general formula (1-3).

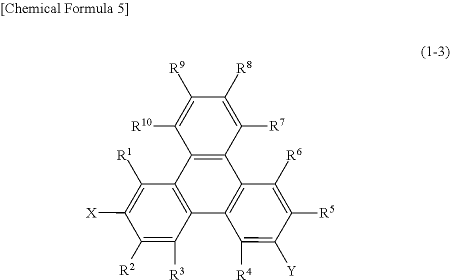

In the formula, X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group. Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group. R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

5) The present invention is the compound having a triphenylene ring structure according to 1), the compound being represented by the following general formula (1-4).

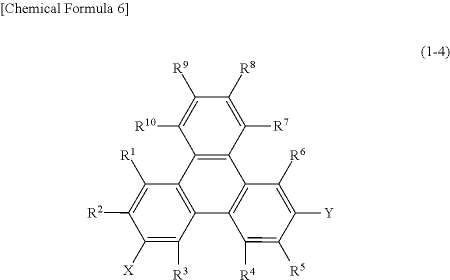

In the formula, X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group. Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group. R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

6) The present invention is the compound having a triphenylene ring structure according to any one of 1) to 5), wherein X in the general formula (1), (1-1), (1-2), (1-3), or (1-4) is a monovalent group selected from substituted or unsubstituted phenoxazinyl, substituted or unsubstituted phenothiazinyl, substituted or unsubstituted acridinyl, substituted or unsubstituted phenazinyl, and carbazolyl having, as a substituent, a disubstituted amino group substituted with an aromatic hydrocarbon group or a condensed polycyclic aromatic group.

7) The present invention is the compound having a triphenylene ring structure according to any one of 1) to 5), wherein Y in the general formula (1), (1-1), (1-2), (1-3), or (1-4) is a monovalent group selected from substituted or unsubstituted phenoxazinyl, substituted or unsubstituted phenothiazinyl, substituted or unsubstituted acridinyl, substituted or unsubstituted phenazinyl, and carbazolyl having, as a substituent, a disubstituted amino group substituted with an aromatic hydrocarbon group or a condensed polycyclic aromatic group.

8) The present invention is the compound having a triphenylene ring structure according to any one of 1) to 5), wherein X and Y in the general formula (1), (1-1), (1-2), (1-3), or (1-4) are a monovalent group selected from substituted or unsubstituted phenoxazinyl, substituted or unsubstituted phenothiazinyl, substituted or unsubstituted acridinyl, substituted or unsubstituted phenazinyl, and carbazolyl having, as a substituent, a disubstituted amino group substituted with an aromatic hydrocarbon group or a condensed polycyclic aromatic group.

9) The present invention is the compound having a triphenylene ring structure according to any one of 1) to 6), wherein Y in the general formula (1), (1-1), (1-2), (1-3), or (1-4) is a hydrogen atom or a deuterium atom.

10) The present invention is a light-emitting material including the compound having a triphenylene ring structure according to any one of 1) to 9).

11) The present invention is the light-emitting material according to 10) that emits delayed fluorescence.

12) The present invention is an organic electroluminescent device that includes a pair of electrodes, and one or more organic layers sandwiched between the pair of electrodes, wherein the compound having a triphenylene ring structure according to any one of 1) to 9) is used as a constituent material of at least one organic layer.

13) The present invention is the organic electroluminescent device according to 12) in which the organic layer produced with the compound having a triphenylene ring structure is a light emitting layer.

14) The present invention is the organic electroluminescent device according to 12) or 13) in which the organic layer produced with the compound having a triphenylene ring structure emits delayed fluorescence.

15) The present invention is the organic electroluminescent device according to 12) in which the organic layer produced with the compound having a triphenylene ring structure is an electron transport layer.

16) The present invention is the organic electroluminescent device according to 12) in which the organic layer produced with the compound having a triphenylene ring structure is a hole blocking layer.

Specific examples of the “aromatic hydrocarbon group”, the “aromatic heterocyclic group”, or the “condensed polycyclic aromatic group” in the “substituted or unsubstituted aromatic hydrocarbon group”, the “substituted or unsubstituted aromatic heterocyclic group”, or the “substituted or unsubstituted condensed polycyclic aromatic group” represented by X in the general formula (1) include phenyl, biphenylyl, terphenylyl, naphthyl, anthryl, phenanthryl, fluorenyl, indenyl, pyrenyl, perylenyl, fluoranthenyl, triphenylenyl, pyridyl, furyl, pyrrolyl, thienyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzoazepinyl, dibenzofuranyl, dibenzothienyl, acridinyl, phenazinyl, phenoxazinyl, phenoselenazinyl, phenothiazinyl, phenotellurazinyl, phenophosphazinyl, and carbolinyl.

Specific examples of the “substituent” in the “substituted aromatic hydrocarbon group”, the “substituted aromatic heterocyclic group”, or the “substituted condensed polycyclic aromatic group” represented by X in the general formula (1) include a deuterium atom; cyano; nitro; halogen atoms such as a fluorine atom, a chlorine atom, a bromine atom, and an iodine atom; linear or branched alkyls of 1 to 6 carbon atoms such as methyl, ethyl, n-propyl, isopropyl, n-butyl, isobutyl, tert-butyl, n-pentyl, isopentyl, neopentyl, and n-hexyl; linear or branched alkyloxy of 1 to 6 carbon atoms such as methyloxy, ethyloxy, and propyloxy; alkenyls such as allyl; aryloxys such as phenyloxy and tolyloxy; arylalkyloxys such as benzyloxy and phenethyloxy; aromatic hydrocarbon groups or condensed polycyclic aromatic groups such as phenyl, biphenylyl, terphenylyl, naphthyl, anthracenyl, phenanthryl, fluorenyl, indenyl, pyrenyl, perylenyl, fluoranthenyl, and triphenylenyl; aromatic heterocyclic groups such as pyridyl, thienyl, furyl, pyrrolyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzofuranyl, dibenzothienyl, phenoxazinyl, phenothiazinyl, and carbolinyl; arylvinyls such as styryl and naphthylvinyl; acyls such as acetyl and benzoyl; dialkylamino groups such as dimethylamino and diethylamino; disubstituted amino groups such as diphenylamino and dinaphthylamino, substituted with aromatic hydrocarbon groups or condensed polycyclic aromatic groups; diaralkylamino groups such as dibenzylamino and diphenethylamino; disubstituted amino groups such as dipyridylamino and dithienylamino, substituted with aromatic heterocyclic groups; dialkenylamino groups such as diallylamino; and disubstituted amino groups substituted with a substituent selected from alkyl, an aromatic hydrocarbon group, a condensed polycyclic aromatic group, aralkyl, an aromatic heterocyclic group, and alkenyl. These substituents may be further substituted with the exemplified substituents above. These substituents may bind to each other via a single bond, substituted or unsubstituted methylene, an oxygen atom, or a sulfur atom to form a ring.

Examples of the “aromatic hydrocarbon group”, the “aromatic heterocyclic group”, or the “condensed polycyclic aromatic group” in the “disubstituted amino group substituted with a group selected from an aromatic hydrocarbon group, an aromatic heterocyclic group, and a condensed polycyclic aromatic group” represented by X in the general formula (1) include the same groups exemplified as the groups for the “aromatic hydrocarbon group”, the “aromatic heterocyclic group”, or the “condensed polycyclic aromatic group” in the “substituted or unsubstituted aromatic hydrocarbon group”, the “substituted or unsubstituted aromatic heterocyclic group”, or the “substituted or unsubstituted condensed polycyclic aromatic group” represented by X in the general formula (1). These groups may have a substituent, and examples of the substituent include the same substituents exemplified as the “substituent” in the “substituted aromatic hydrocarbon group”, the “substituted aromatic heterocyclic group”, or the “substituted condensed polycyclic aromatic group” represented by X in the general formula (1), and possible embodiments may also be the same embodiments as the exemplified embodiments.

X in the general formula (1) is preferably the “substituted or unsubstituted aromatic heterocyclic group” or the “substituted or unsubstituted condensed polycyclic aromatic group”, far preferably, the “substituted or unsubstituted aromatic heterocyclic group”, particularly preferably, phenoxazinyl, phenothiazinyl, acridinyl, phenazinyl, or carbazolyl having, as a substituent, a disubstituted amino group substituted with an aromatic hydrocarbon group or a condensed polycyclic aromatic group, even more particularly preferably, a phenoxazin-10-yl group, a phenothiazin-10-yl group, a 9,9-dimethylacridan-10-yl group, 10-phenylphenazin-9-yl group, or carbazolyl having diphenylamino as a substituent.

A substituent for these groups is preferably carbazolyl or a disubstituted amino group substituted with an aromatic hydrocarbon group, far preferably, carbazolyl or diphenylamino.

Specific examples of the “linear or branched alkyl of 1 to 6 carbon atoms”, the “cycloalkyl of 5 to 10 carbon atoms”, or the “linear or branched alkenyl of 2 to 6 carbon atoms” in the “linear or branched alkyl of 1 to 6 carbon atoms that may have a substituent”, the “cycloalkyl of 5 to 10 carbon atoms that may have a substituent”, or the “linear or branched alkenyl of 2 to 6 carbon atoms that may have a substituent” represented by Y in the general formula (1) include methyl, ethyl, n-propyl, isopropyl, n-butyl, isobutyl, tert-butyl, n-pentyl, isopentyl, neopentyl, n-hexyl, cyclopentyl, cyclohexyl, 1-adamantyl, 2-adamantyl, vinyl, allyl, isopropenyl, and 2-butenyl. These groups may bind to each other via a single bond, substituted or unsubstituted methylene, an oxygen atom, or a sulfur atom to form a ring.

Specific examples of the “substituent” in the “linear or branched alkyl of 1 to 6 carbon atoms that has a substituent”, the “cycloalkyl of 5 to 10 carbon atoms that has a substituent”, or the “linear or branched alkenyl of 2 to 6 carbon atoms that has a substituent” represented by Y in the general formula (1) include a deuterium atom; cyano; nitro; halogen atoms such as a fluorine atom, a chlorine atom, a bromine atom, and an iodine atom; linear or branched alkyloxys of 1 to 6 carbon atoms such as methyloxy, ethyloxy, and propyloxy; alkenyls such as allyl; aryloxys such as phenyloxy and tolyloxy; arylalkyloxys such as benzyloxy and phenethyloxy; aromatic hydrocarbon groups or condensed polycyclic aromatic groups such as phenyl, biphenylyl, terphenylyl, naphthyl, anthracenyl, phenanthryl, fluorenyl, indenyl, pyrenyl, perylenyl, fluoranthenyl, and triphenylenyl; and aromatic heterocyclic groups such as pyridyl, thienyl, furyl, pyrrolyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzofuranyl, dibenzothienyl, and carbolinyl. These substituents may be further substituted with the exemplified substituents above. These substituents may bind to each other via a single bond, substituted or unsubstituted methylene, an oxygen atom, or a sulfur atom to form a ring.

Specific examples of the “linear or branched alkyloxy of 1 to 6 carbon atoms” or the “cycloalkyloxy of 5 to 10 carbon atoms” in the “linear or branched alkyloxy of 1 to 6 carbon atoms that may have a substituent” or the “cycloalkyloxy of 5 to 10 carbon atoms that may have a substituent” represented by Y in the general formula (1) include methyloxy, ethyloxy, n-propyloxy, isopropyloxy, n-butyloxy, tert-butyloxy, n-pentyloxy, n-hexyloxy, cyclopentyloxy, cyclohexyloxy, cycloheptyloxy, cyclooctyloxy, 1-adamantyloxy, and 2-adamantyloxy. These groups may bind to each other via a single bond, substituted or unsubstituted methylene, an oxygen atom, or a sulfur atom to form a ring.

Specific examples of the “substituent” in the “linear or branched alkyloxy of 1 to 6 carbon atoms that has a substituent” or the “cycloalkyloxy of 5 to 10 carbon atoms that has a substituent” represented by Y in the general formula (1) include a deuterium atom; cyano; nitro; halogen atoms such as a fluorine atom, a chlorine atom, a bromine atom, and an iodine atom; linear or branched alkyloxys of 1 to 6 carbon atoms such as methyloxy, ethyloxy, and propyloxy; alkenyls such as allyl; aryloxys such as phenyloxy and tolyloxy; arylalkyloxys such as benzyloxy and phenethyloxy; aromatic hydrocarbon groups or condensed polycyclic aromatic groups such as phenyl, biphenylyl, terphenylyl, naphthyl, anthracenyl, phenanthryl, fluorenyl, indenyl, pyrenyl, perylenyl, fluoranthenyl, and triphenylenyl; and aromatic heterocyclic groups such as pyridyl, thienyl, furyl, pyrrolyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzofuranyl, dibenzothienyl, and carbolinyl. These substituents may be further substituted with the exemplified substituents above. These substituents may bind to each other via a single bond, substituted or unsubstituted methylene, an oxygen atom, or a sulfur atom to form a ring.

Examples of the “aromatic hydrocarbon group”, the “aromatic heterocyclic group”, or the “condensed polycyclic aromatic group” in the “substituted or unsubstituted aromatic hydrocarbon group”, the “substituted or unsubstituted aromatic heterocyclic group”, or the “substituted or unsubstituted condensed polycyclic aromatic group” represented by Y in the general formula (1) include the same groups exemplified as the groups for the “aromatic hydrocarbon group”, the “aromatic heterocyclic group”, or the “condensed polycyclic aromatic group” in the “substituted or unsubstituted aromatic hydrocarbon group”, the “substituted or unsubstituted aromatic heterocyclic group”, or the “substituted or unsubstituted condensed polycyclic aromatic group” represented by X in the general formula (1). These groups may have a substituent, and examples of the substituent include the same substituents exemplified as the “substituent” in the “substituted aromatic hydrocarbon group”, the “substituted aromatic heterocyclic group”, or the “substituted condensed polycyclic aromatic group” represented by X in the general formula (1), and possible embodiments may also be the same embodiments as the exemplified embodiments.

Specific examples of the “aryloxy” in the “substituted or unsubstituted aryloxy” represented by Y in the general formula (1) include phenyloxy, biphenylyloxy, terphenylyloxy, naphthyloxy, anthryloxy, phenanthryloxy, fluorenyloxy, indenyloxy, pyrenyloxy, and perylenyloxy.

Specific examples of the “substituent” in the “substituted aryloxy” represented by Y in the general formula (1) include a deuterium atom; trifluoromethyl; cyano; nitro; halogen atoms such as a fluorine atom, a chlorine atom, a bromine atom, and an iodine atom; linear or branched alkyls of 1 to 6 carbon atoms such as methyl, ethyl, n-propyl, isopropyl, n-butyl, isobutyl, tert-butyl, n-pentyl, isopentyl, neopentyl, and n-hexyl; linear or branched alkyloxys of 1 to 6 carbon atoms such as methyloxy, ethyloxy, and propyloxy; alkenyls such as allyl; aralkyls such as benzyl, naphthylmethyl, and phenethyl; aryloxys such as phenyloxy and tolyloxy; arylalkyloxys such as benzyloxy and phenethyloxy; aromatic hydrocarbon groups or condensed polycyclic aromatic groups such as phenyl, biphenylyl, terphenylyl, naphthyl, anthracenyl, phenanthryl, fluorenyl, indenyl, pyrenyl, perylenyl, fluoranthenyl, and triphenylenyl; aromatic heterocyclic groups such as pyridyl, thienyl, furyl, pyrrolyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzofuranyl, dibenzothienyl, and carbolinyl; arylvinyls such as styryl and naphthylvinyl; acyls such as acetyl and benzoyl; dialkylamino groups such as dimethylamino and diethylamino; disubstituted amino groups such as diphenylamino and dinaphthylamino, substituted with aromatic hydrocarbon groups or condensed polycyclic aromatic groups; diaralkylamino groups such as dibenzylamino and diphenethylamino; disubstituted amino groups such as dipyridylamino and dithienylamino, substituted with aromatic heterocyclic groups; dialkenylamino groups such as diallylamino; and disubstituted amino groups substituted with a substituent selected from alkyl, an aromatic hydrocarbon group, a condensed polycyclic aromatic group, aralkyl, an aromatic heterocyclic group, and alkenyl. These substituents may be further substituted with the exemplified substituents above. These substituents may bind to each other via a single bond, substituted or unsubstituted methylene, an oxygen atom, or a sulfur atom to form a ring.

Examples of the “aromatic hydrocarbon group”, the “aromatic heterocyclic group”, or the “condensed polycyclic aromatic group” in the “disubstituted amino group substituted with a group selected from an aromatic hydrocarbon group, an aromatic heterocyclic group, and a condensed polycyclic aromatic group” represented by Y in the general formula (1) include the same groups exemplified as the groups for the “aromatic hydrocarbon group”, the “aromatic heterocyclic group”, or the “condensed polycyclic aromatic group” in the “substituted or unsubstituted aromatic hydrocarbon group”, the “substituted or unsubstituted aromatic heterocyclic group”, or the “substituted or unsubstituted condensed polycyclic aromatic group” represented by X in the general formula (1). These groups may have a substituent, and examples of the substituent include the same substituents exemplified as the “substituent” in the “substituted aromatic hydrocarbon group”, the “substituted aromatic heterocyclic group”, or the “substituted condensed polycyclic aromatic group” represented by X in the general formula (1), and possible embodiments may also be the same embodiments as the exemplified embodiments.

Y in the general formula (1) is preferably a hydrogen atom, the “substituted or unsubstituted aromatic heterocyclic group”, or the “substituted or unsubstituted condensed polycyclic aromatic group”, far preferably, a hydrogen atom or the “substituted or unsubstituted aromatic heterocyclic group”, particularly preferably, phenoxazinyl, phenothiazinyl, acridinyl, phenazinyl, or carbazolyl having, as a substituent, a disubstituted amino group substituted with an aromatic hydrocarbon group or a condensed polycyclic aromatic group, even more particularly preferably, a phenoxazin-10-yl group, a phenothiazin-10-yl group, a 9,9-dimethylacridan-10-yl group, 10-phenylphenazin-9-yl group, or carbazolyl having diphenylamino as a substituent.

A substituent for these groups is preferably carbazolyl or a disubstituted amino group substituted with an aromatic hydrocarbon group, far preferably, carbazolyl or diphenylamino.

Specific examples of the “linear or branched alkyl of 1 to 6 carbon atoms”, the “cycloalkyl of 5 to 10 carbon atoms”, or the “linear or branched alkenyl of 2 to 6 carbon atoms” in the “linear or branched alkyl of 1 to 6 carbon atoms that may have a substituent”, the “cycloalkyl of 5 to 10 carbon atoms that may have a substituent”, or the “linear or branched alkenyl of 2 to 6 carbon atoms that may have a substituent” represented by R¹ to R¹⁰ in the general formula (1) include methyl, ethyl, n-propyl, isopropyl, n-butyl, isobutyl, tert-butyl, n-pentyl, isopentyl, neopentyl, n-hexyl, cyclopentyl, cyclohexyl, 1-adamantyl, 2-adamantyl, vinyl, allyl, isopropenyl, and 2-butenyl. These groups may bind to each other via a single bond, substituted or unsubstituted methylene, an oxygen atom, or a sulfur atom to form a ring.

Specific examples of the “substituent” in the “linear or branched alkyl of 1 to 6 carbon atoms that has a substituent”, the “cycloalkyl of 5 to 10 carbon atoms that has a substituent”, or the “linear or branched alkenyl of 2 to 6 carbon atoms that has a substituent” represented by R¹ to R¹⁰ in the general formula (1) include a deuterium atom; cyano; nitro; halogen atoms such as a fluorine atom, a chlorine atom, a bromine atom, and an iodine atom; linear or branched alkyloxys of 1 to 6 carbon atoms such as methyloxy, ethyloxy, and propyloxy; alkenyls such as allyl; aryloxys such as phenyloxy and tolyloxy; arylalkyloxys such as benzyloxy and phenethyloxy; aromatic hydrocarbon groups or condensed polycyclic aromatic groups such as phenyl, biphenylyl, terphenylyl, naphthyl, anthracenyl, phenanthryl, fluorenyl, indenyl, pyrenyl, perylenyl, fluoranthenyl, and triphenylenyl; and aromatic heterocyclic groups such as pyridyl, thienyl, furyl, pyrrolyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzofuranyl, dibenzothienyl, and carbolinyl. These substituents may be further substituted with the exemplified substituents above. These substituents may bind to each other via a single bond, substituted or unsubstituted methylene, an oxygen atom, or a sulfur atom to form a ring.

Specific examples of the “linear or branched alkyloxy of 1 to 6 carbon atoms” or the “cycloalkyloxy of 5 to 10 carbon atoms” in the “linear or branched alkyloxy of 1 to 6 carbon atoms that may have a substituent” or the “cycloalkyloxy of 5 to 10 carbon atoms that may have a substituent” represented by R¹ to R¹⁰ in the general formula (1) include methyloxy, ethyloxy, n-propyloxy, isopropyloxy, n-butyloxy, tert-butyloxy, n-pentyloxy, n-hexyloxy, cyclopentyloxy, cyclohexyloxy, cycloheptyloxy, cyclooctyloxy, 1-adamantyloxy, and 2-adamantyloxy. These groups may bind to each other via a single bond, substituted or unsubstituted methylene, an oxygen atom, or a sulfur atom to form a ring.

Specific examples of the “substituent” in the “linear or branched alkyloxy of 1 to 6 carbon atoms that has a substituent” or the “cycloalkyloxy of 5 to 10 carbon atoms that has a substituent” represented by R¹ to R¹⁰ in the general formula (1) include a deuterium atom; cyano; nitro; halogen atoms such as a fluorine atom, a chlorine atom, a bromine atom, and an iodine atom; linear or branched alkyloxys of 1 to 6 carbon atoms such as methyloxy, ethyloxy, and propyloxy; alkenyls such as allyl; aryloxys such as phenyloxy and tolyloxy; arylalkyloxys such as benzyloxy and phenethyloxy; aromatic hydrocarbon groups or condensed polycyclic aromatic groups such as phenyl, biphenylyl, terphenylyl, naphthyl, anthracenyl, phenanthryl, fluorenyl, indenyl, pyrenyl, perylenyl, fluoranthenyl, and triphenylenyl; and aromatic heterocyclic groups such as pyridyl, thienyl, furyl, pyrrolyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzofuranyl, dibenzothienyl, and carbolinyl. These substituents may be further substituted with the exemplified substituents above. These substituents may bind to each other via a single bond, substituted or unsubstituted methylene, an oxygen atom, or a sulfur atom to form a ring.

Specific examples of the “aromatic hydrocarbon group”, the “aromatic heterocyclic group”, or the “condensed polycyclic aromatic group” in the “substituted or unsubstituted aromatic hydrocarbon group”, the “substituted or unsubstituted aromatic heterocyclic group”, or the “substituted or unsubstituted condensed polycyclic aromatic group” represented by R¹ to R¹⁰ in the general formula (1) include phenyl, biphenylyl, terphenylyl, naphthyl, anthryl, phenanthryl, fluorenyl, indenyl, pyrenyl, perylenyl, fluoranthenyl, triphenylenyl, pyridyl, furyl, pyrrolyl, thienyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzofuranyl, dibenzothienyl, phenoxazinyl, phenothiazinyl, and carbolinyl. These groups may bind to each other via a single bond, substituted or unsubstituted methylene, an oxygen atom, or a sulfur atom to form a ring.

Specific examples of the “substituent” in the “substituted aromatic hydrocarbon group”, the “substituted aromatic heterocyclic group”, or the “substituted condensed polycyclic aromatic group” represented by R¹ to R¹⁰ in the general formula (1) include a deuterium atom; trifluoromethyl; cyano; nitro; halogen atoms such as a fluorine atom, a chlorine atom, a bromine atom, and an iodine atom; linear or branched alkyls of 1 to 6 carbon atoms such as methyl, ethyl, n-propyl, isopropyl, n-butyl, isobutyl, tert-butyl, n-pentyl, isopentyl, neopentyl, and n-hexyl; linear or branched alkyloxys of 1 to 6 carbon atoms such as methyloxy, ethyloxy, and propyloxy; alkenyls such as allyl; aralkyls such as benzyl, naphthylmethyl, and phenethyl; aryloxys such as phenyloxy and tolyloxy; arylalkyloxys such as benzyloxy and phenethyloxy; aromatic hydrocarbon groups or condensed polycyclic aromatic groups such as phenyl, biphenylyl, terphenylyl, naphthyl, anthracenyl, phenanthryl, fluorenyl, indenyl, pyrenyl, perylenyl, fluoranthenyl, and triphenylenyl; aromatic heterocyclic groups such as pyridyl, thienyl, furyl, pyrrolyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzofuranyl, dibenzothienyl, phenoxazinyl, phenothiazinyl, and carbolinyl; arylvinyls such as styryl and naphthylvinyl; acyls such as acetyl and benzoyl; dialkylamino groups such as dimethylamino and diethylamino; disubstituted amino groups such as diphenylamino and dinaphthylamino, substituted with aromatic hydrocarbon groups or condensed polycyclic aromatic groups; diaralkylamino groups such as dibenzylamino and diphenethylamino; disubstituted amino groups such as dipyridylamino and dithienylamino, substituted with aromatic heterocyclic groups; dialkenylamino groups such as diallylamino; and disubstituted amino groups substituted with a substituent selected from alkyl, an aromatic hydrocarbon group, a condensed polycyclic aromatic group, aralkyl, an aromatic heterocyclic group, and alkenyl. These substituents may be further substituted with the exemplified substituents above. These substituents may bind to each other via a single bond, substituted or unsubstituted methylene, an oxygen atom, or a sulfur atom to form a ring.

Specific examples of the “aryloxy” in the “substituted or unsubstituted aryloxy” represented by R¹ to R¹⁰ in the general formula (1) include phenyloxy, biphenylyloxy, terphenylyloxy, naphthyloxy, anthryloxy, phenanthryloxy, fluorenyloxy, indenyloxy, pyrenyloxy, and perylenyloxy. These groups may bind to each other via a single bond, substituted or unsubstituted methylene, an oxygen atom, or a sulfur atom to form a ring.

Specific examples of the “substituent” in the “substituted aryloxy” represented by R¹ to R¹⁰ in the general formula (1) include a deuterium atom; trifluoromethyl; cyano; nitro; halogen atoms such as a fluorine atom, a chlorine atom, a bromine atom, and an iodine atom; linear or branched alkyls of 1 to 6 carbon atoms such as methyl, ethyl, n-propyl, isopropyl, n-butyl, isobutyl, tert-butyl, n-pentyl, isopentyl, neopentyl, and n-hexyl; linear or branched alkyloxys of 1 to 6 carbon atoms such as methyloxy, ethyloxy, and propyloxy; alkenyls such as allyl; aralkyls such as benzyl, naphthylmethyl, and phenethyl; aryloxys such as phenyloxy and tolyloxy; arylalkyloxys such as benzyloxy and phenethyloxy; aromatic hydrocarbon groups or condensed polycyclic aromatic groups such as phenyl, biphenylyl, terphenylyl, naphthyl, anthracenyl, phenanthryl, fluorenyl, indenyl, pyrenyl, perylenyl, fluoranthenyl, and triphenylenyl; aromatic heterocyclic groups such as pyridyl, thienyl, furyl, pyrrolyl, quinolyl, isoquinolyl, benzofuranyl, benzothienyl, indolyl, carbazolyl, benzoxazolyl, benzothiazolyl, quinoxalyl, benzoimidazolyl, pyrazolyl, dibenzofuranyl, dibenzothienyl, and carbolinyl; arylvinyls such as styryl and naphthylvinyl; acyls such as acetyl and benzoyl; dialkylamino groups such as dimethylamino and diethylamino; disubstituted amino groups such as diphenylamino and dinaphthylamino, substituted with aromatic hydrocarbon groups or condensed polycyclic aromatic groups; diaralkylamino groups such as dibenzylamino and diphenethylamino; disubstituted amino groups such as dipyridylamino and dithienylamino, substituted with aromatic heterocyclic groups; dialkenylamino groups such as diallylamino; and disubstituted amino groups substituted with a substituent selected from alkyl, an aromatic hydrocarbon group, a condensed polycyclic aromatic group, aralkyl, an aromatic heterocyclic group, and alkenyl. These substituents may be further substituted with the exemplified substituents above. These substituents may bind to each other via a single bond, substituted or unsubstituted methylene, an oxygen atom, or a sulfur atom to form a ring.

Examples of the “aromatic hydrocarbon group”, the “aromatic heterocyclic group”, or the “condensed polycyclic aromatic group” in the “disubstituted amino group substituted with a group selected from an aromatic hydrocarbon group, an aromatic heterocyclic group, and a condensed polycyclic aromatic group” represented by R¹ to R¹⁰ in the general formula (1) include the same groups exemplified as the groups for the “aromatic hydrocarbon group”, the “aromatic heterocyclic group”, or the “condensed polycyclic aromatic group” in the “substituted or unsubstituted aromatic hydrocarbon group”, the “substituted or unsubstituted aromatic heterocyclic group”, or the “substituted or unsubstituted condensed polycyclic aromatic group” represented by X in the general formula (1). These groups may have a substituent, and examples of the substituent include the same substituents exemplified as the “substituent” in the “substituted aromatic hydrocarbon group”, the “substituted aromatic heterocyclic group”, or the “substituted condensed polycyclic aromatic group” represented by X in the general formula (1), and possible embodiments may also be the same embodiments as the exemplified embodiments.

The compounds of the general formula (1) having a triphenylene ring structure of the present invention can emit delayed fluorescence and have a stable thin-film state as well as high luminous efficiency because of a small difference between excited triplet energy and excited singlet energy (ΔE_{ST}), and a comparatively high oscillator strength (f) which are obtained by theoretical calculation.

The compounds of the general formula (1) having a triphenylene ring structure of the present invention can be used as a constituent material of the light emitting layer of an organic electroluminescent device (hereinafter referred to as an organic EL device). With the use of the compounds of the present invention that emit delayed fluorescence, the luminous efficiency is dramatically improved.

The compounds of the general formula (1) having a triphenylene ring structure of the present invention can be used as a constituent material of the electron transport layer of an organic EL device. The use of the material having higher electron injectability and mobility than the conventional materials has effects of improving the electron transport efficiency from the electron transport layer to the light emitting layer to improve the luminous efficiency while lowering a driving voltage to improve the durability of the organic EL device.

The compounds of the general formula (1) having a triphenylene ring structure of the present invention can also be used as a constituent material of the hole blocking layer of an organic EL device. The use of the material having an excellent hole blocking ability and superior electron transportability and higher stability in the thin-film state than the conventional materials has effects of lowering the driving voltage and improving the current resistance while maintaining high luminous efficiency, thereby improving the maximum emission luminance of the organic EL device.

### Effects of the Invention

The compounds having a triphenylene ring structure of the present invention are useful as a light-emitting material (a dopant compound) of the light emitting layer of an organic EL device or as a constituent material of the electron transport layer or the hole blocking layer of an organic EL device. The compounds can emit delayed fluorescence, have a stable thin-film state and excel in heat resistance. The organic EL device produced by using the compounds can have high efficiency, high luminance, and low driving voltage.

### BRIEF DESCRIPTION OF THE DRAWINGS

FIG. 1 is a ¹H-NMR chart of the compound of Example 1 of the present invention (Compound 1).

FIG. 2 is a ¹H-NMR chart of the compound of Example 2 of the present invention (Compound 5).

FIG. 3 is a ¹H-NMR chart of the compound of Example 3 of the present invention (Compound 20).

FIG. 4 is a ¹H-NMR chart of the compound of Example 4 of the present invention (Compound 21).

FIG. 5 is a ¹H-NMR chart of the compound of Example 5 of the present invention (Compound 24).

FIG. 6 is a diagram illustrating the configuration of the EL device of Example 13.

### MODE FOR CARRYING OUT THE INVENTION

The compounds having a triphenylene ring structure of the present invention may be synthesized by using, for example, the following method. First, a triphenylene derivative having a halogen substituent can be synthesized by a reaction of a 1,1′:2′,1″-terphenyl derivative having a halogen substituent such as bromine or iodine, with molybdenum chloride. The triphenylene derivative having a halogen substituent is reacted with a corresponding boric acid ester synthesized from a corresponding aryl halide in a cross-coupling reaction such as Suzuki coupling (refer to Non-Patent Document 2, for example) or in a condensation reaction such as a Buchwald-Hartwig reaction in order to attain the synthesis of the compound having a triphenylene ring structure of the present invention.

The compounds having a triphenylene ring structure of the present invention may be synthesized also by the following method. First, triphenylene is synthesized by a reaction of a 1,1′:2′,1″-terphenyl derivative with molybdenum chloride, followed by bromination using N-bromosuccinimide or the like to synthesize a triphenylene derivative having a bromo group. The compound having a triphenylene ring structure of the present invention can then be synthesized by conducting a cross-coupling reaction such as Suzuki coupling or a condensation reaction such as a Buchwald-Hartwig reaction in the same manner as mentioned above.

A bromo compound having substituents different in substituted positions and the number of substitution can be obtained by changing reagents and conditions of bromination.

The following presents specific examples of preferred compounds among the compounds of the general formula (1) having a triphenylene ring structure. The present invention, however, is not restricted to these compounds.

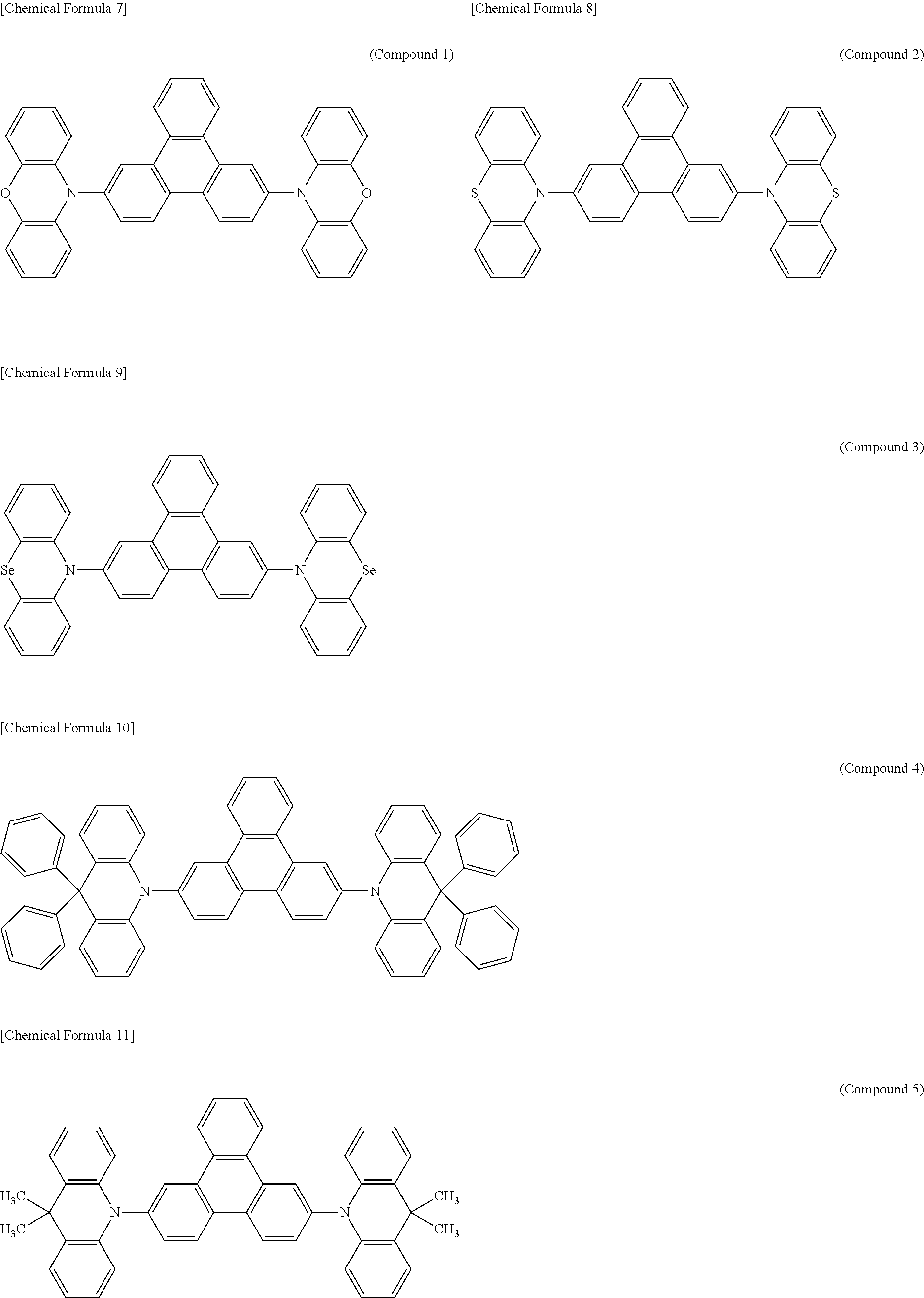

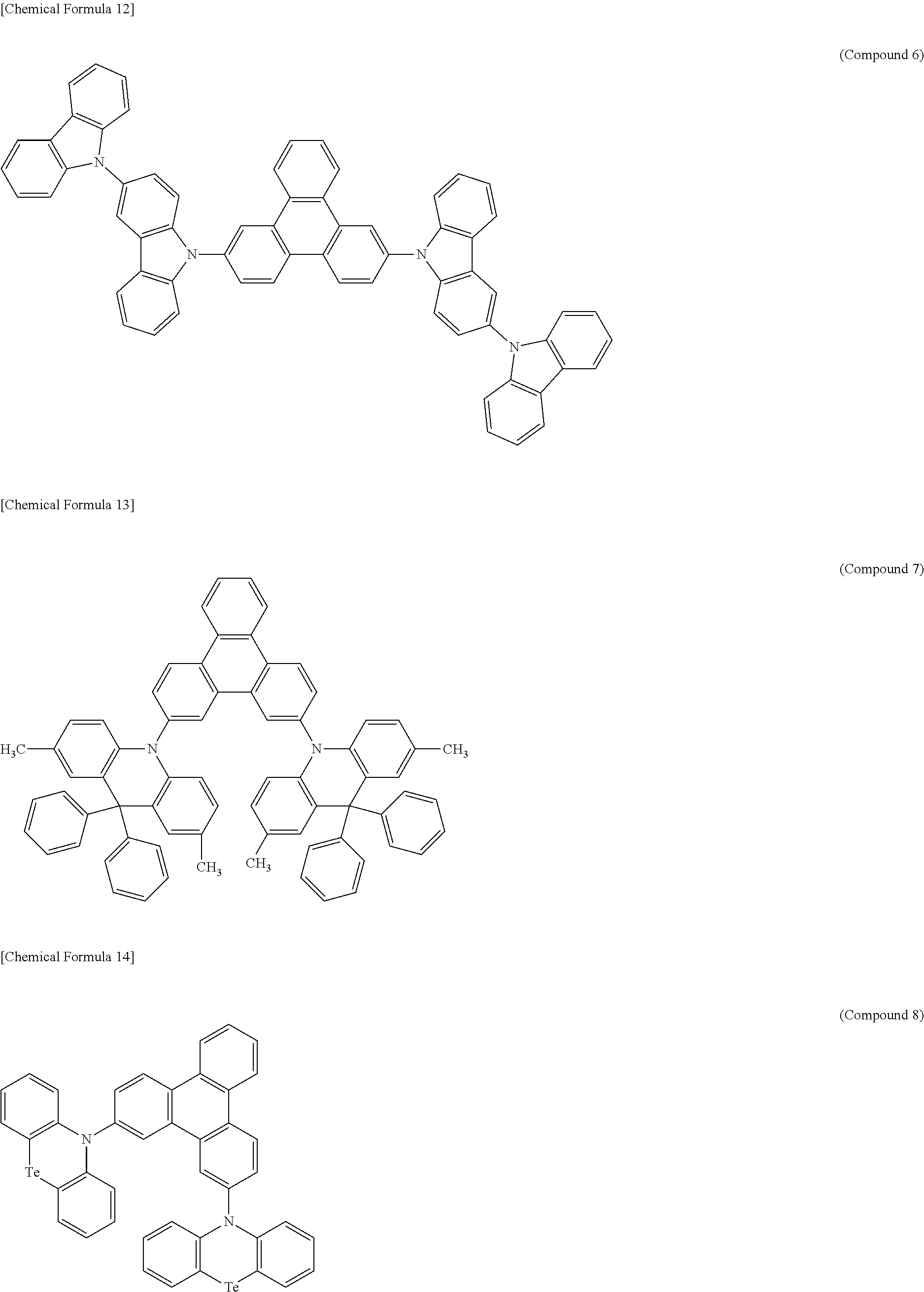

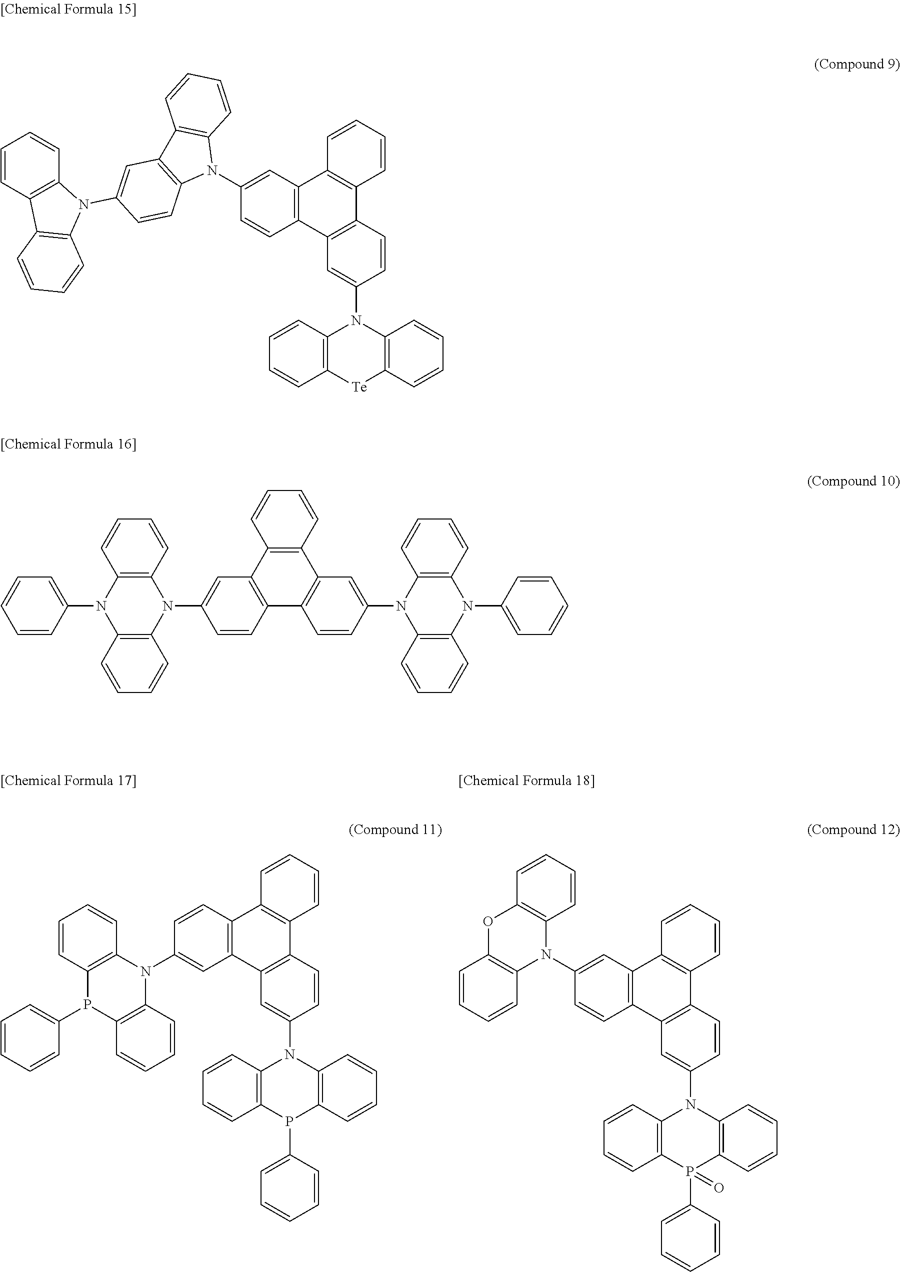

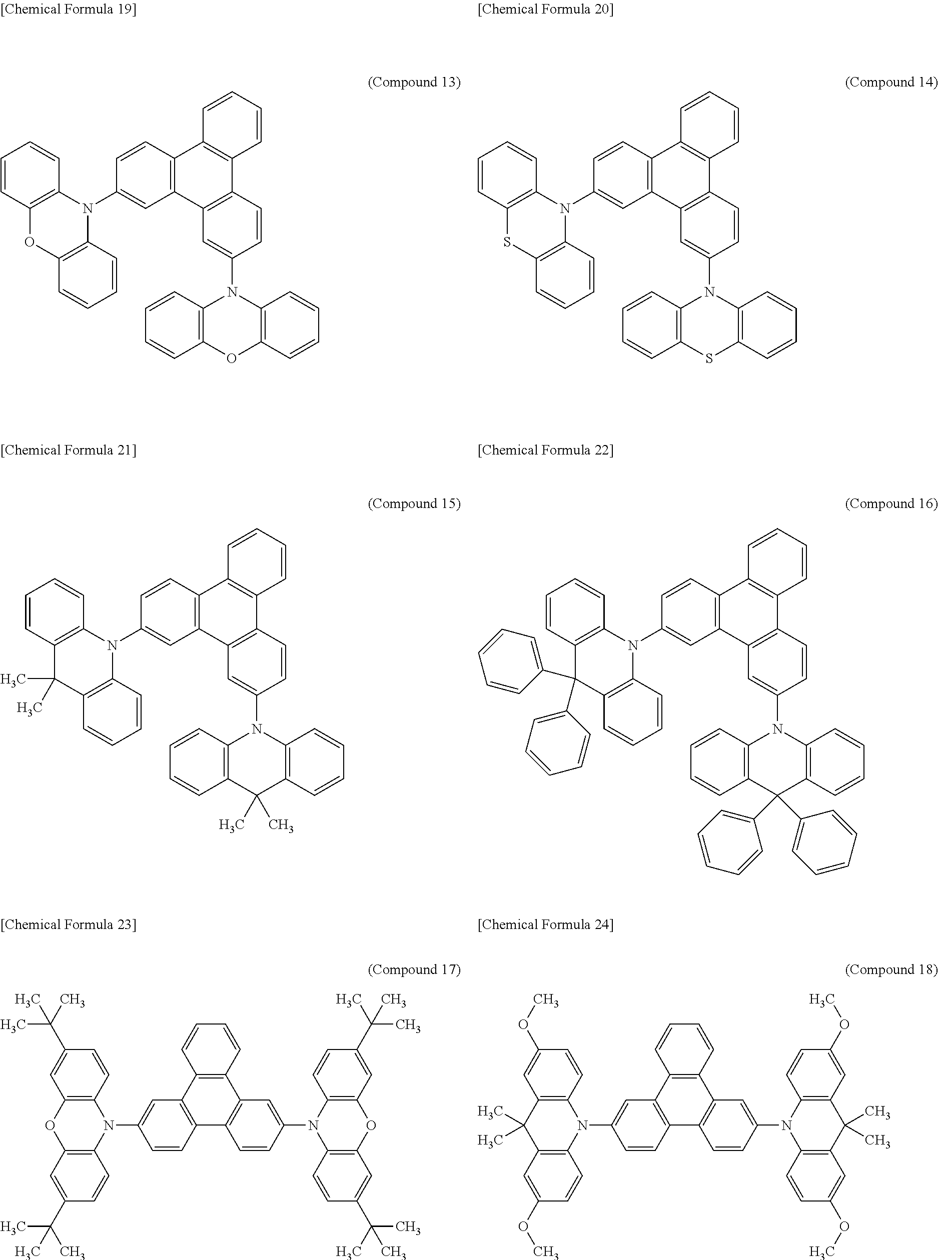

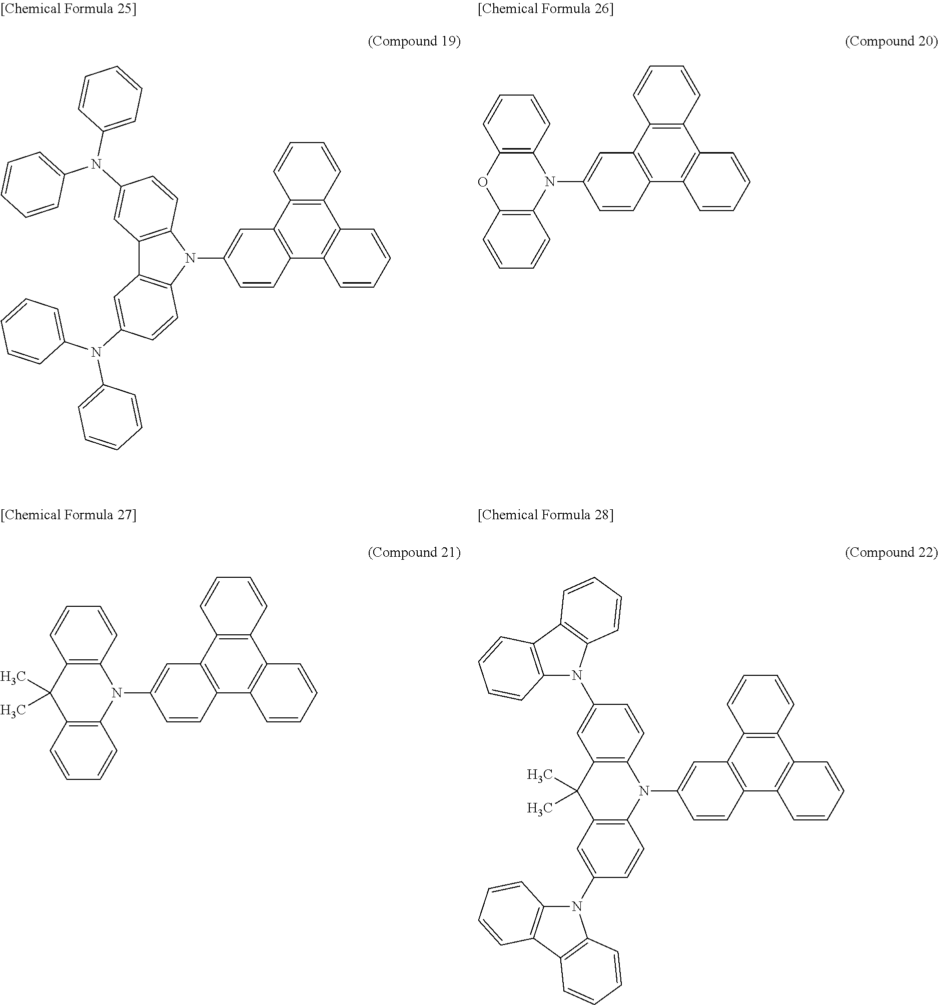

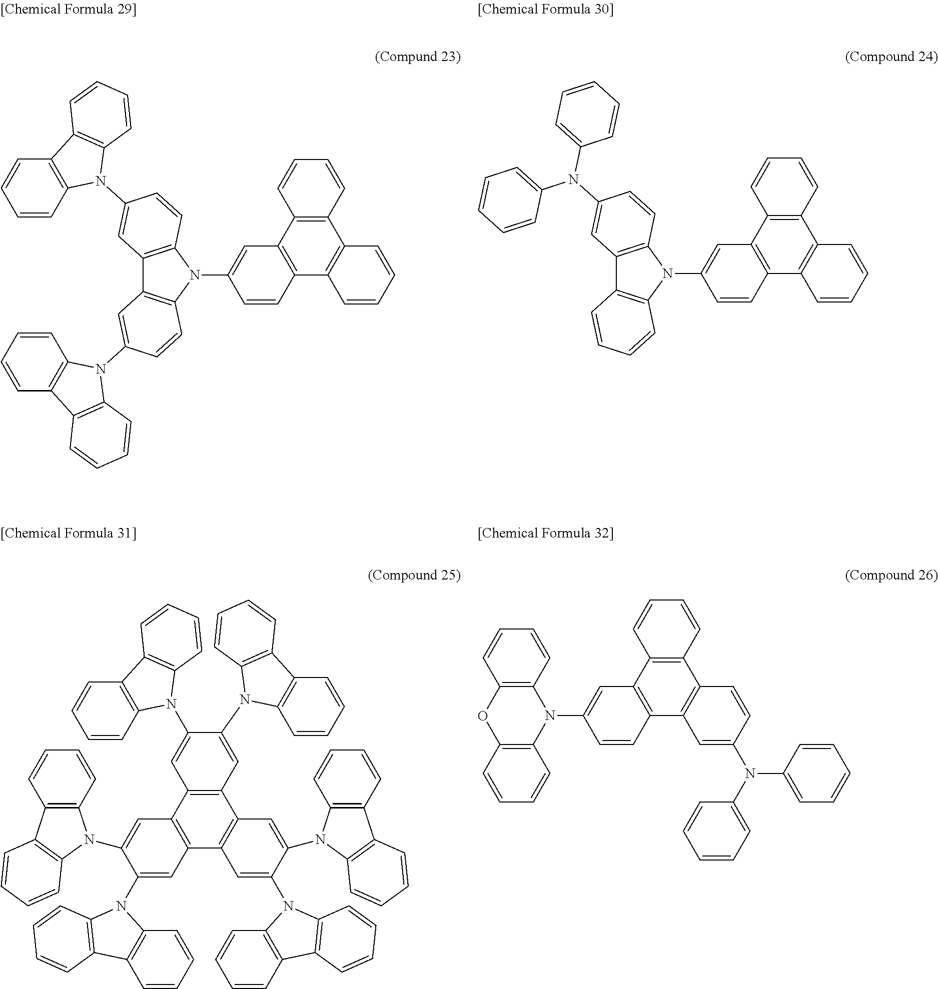

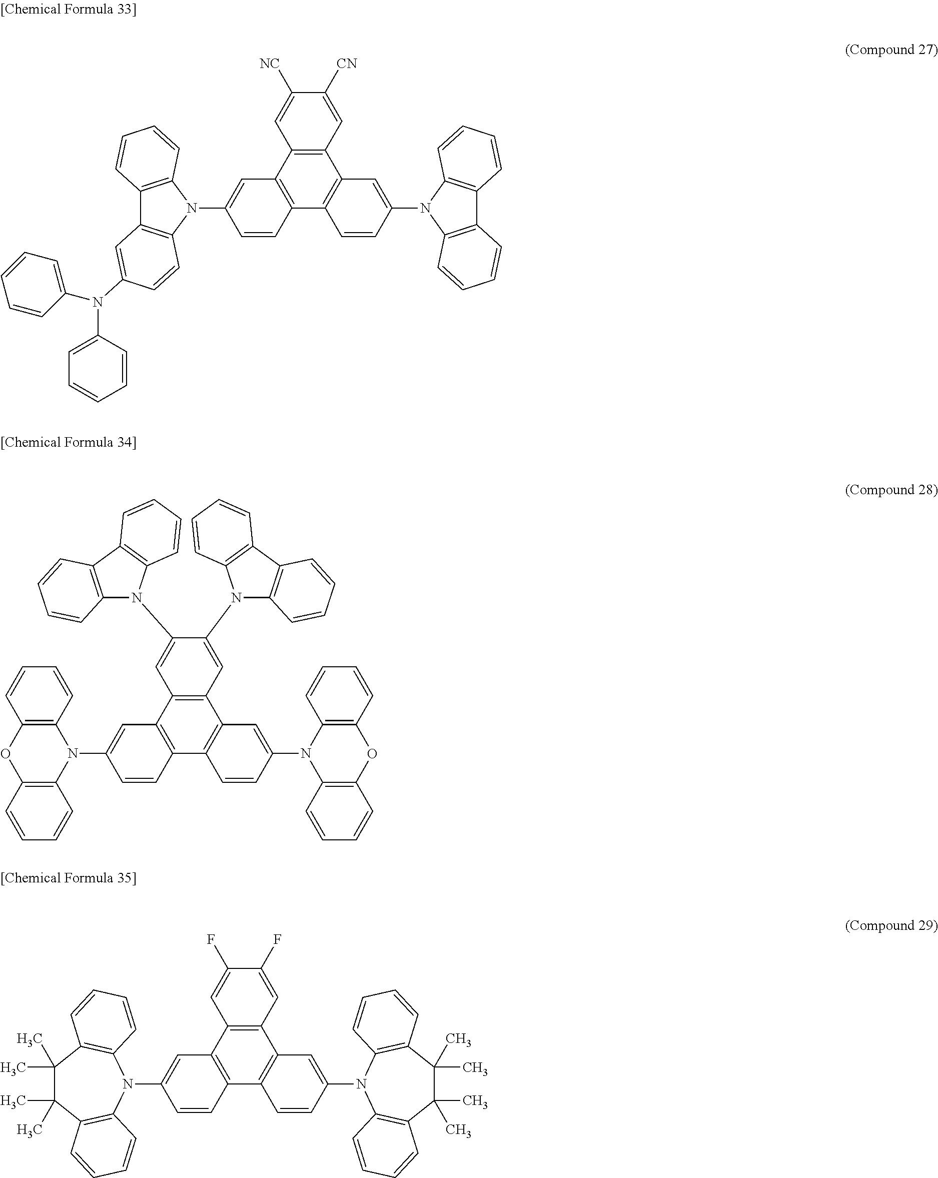

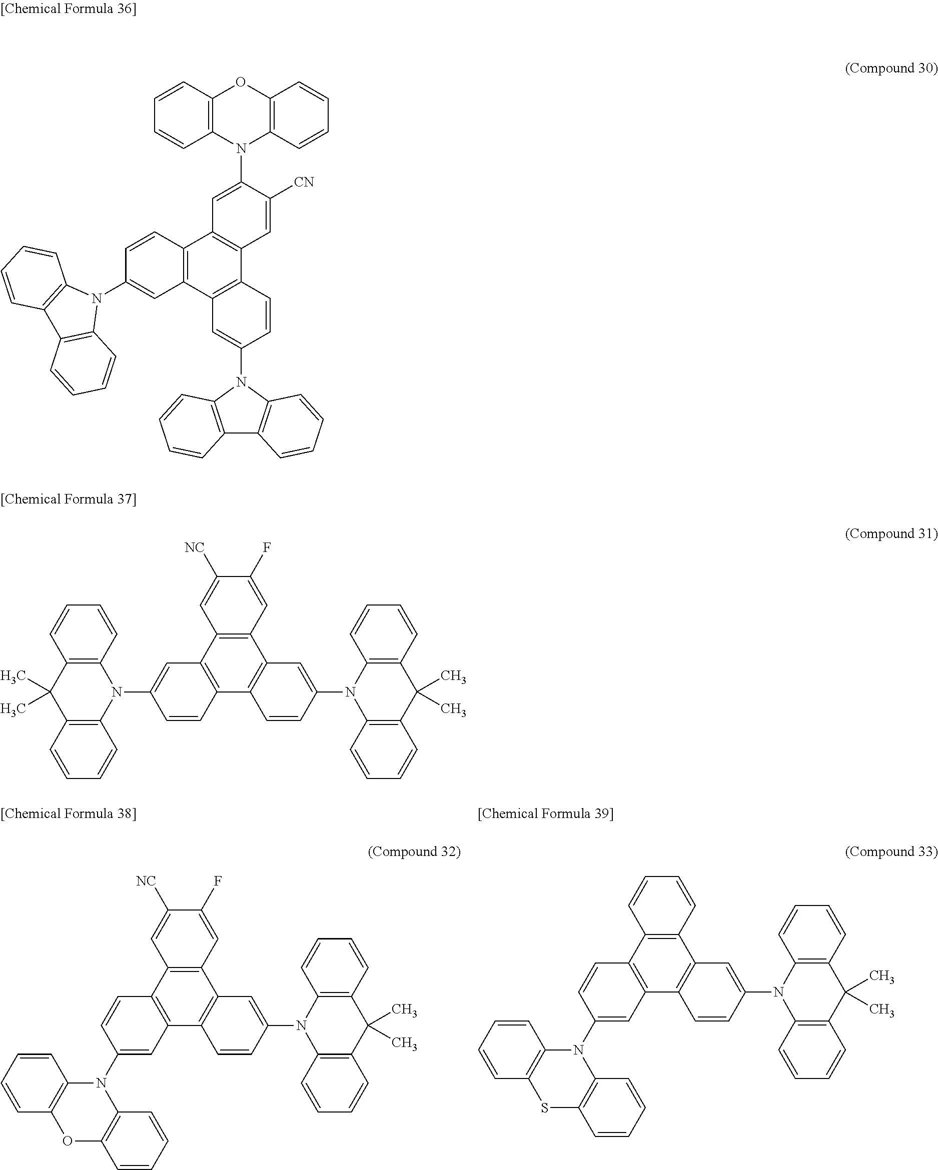

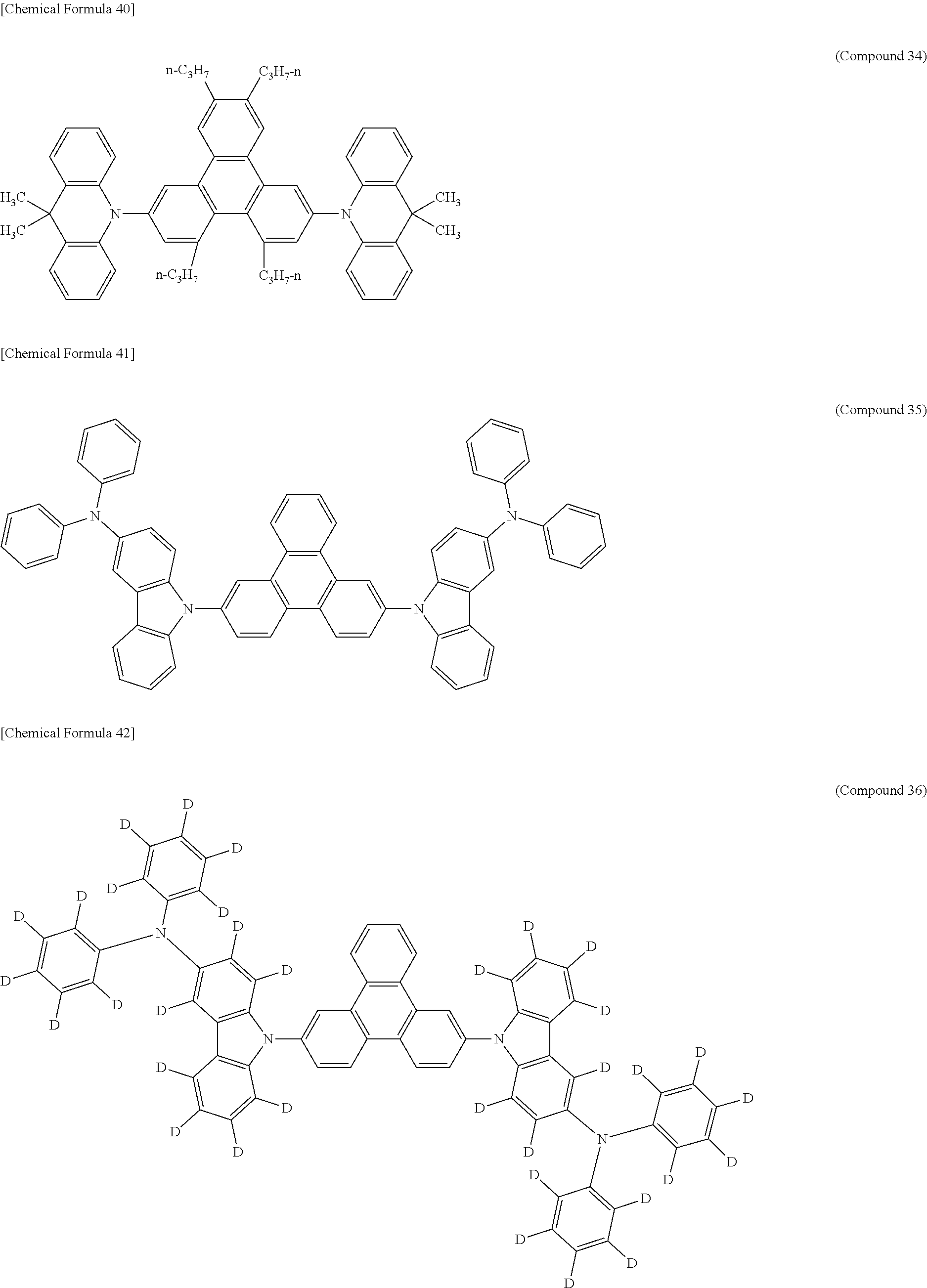

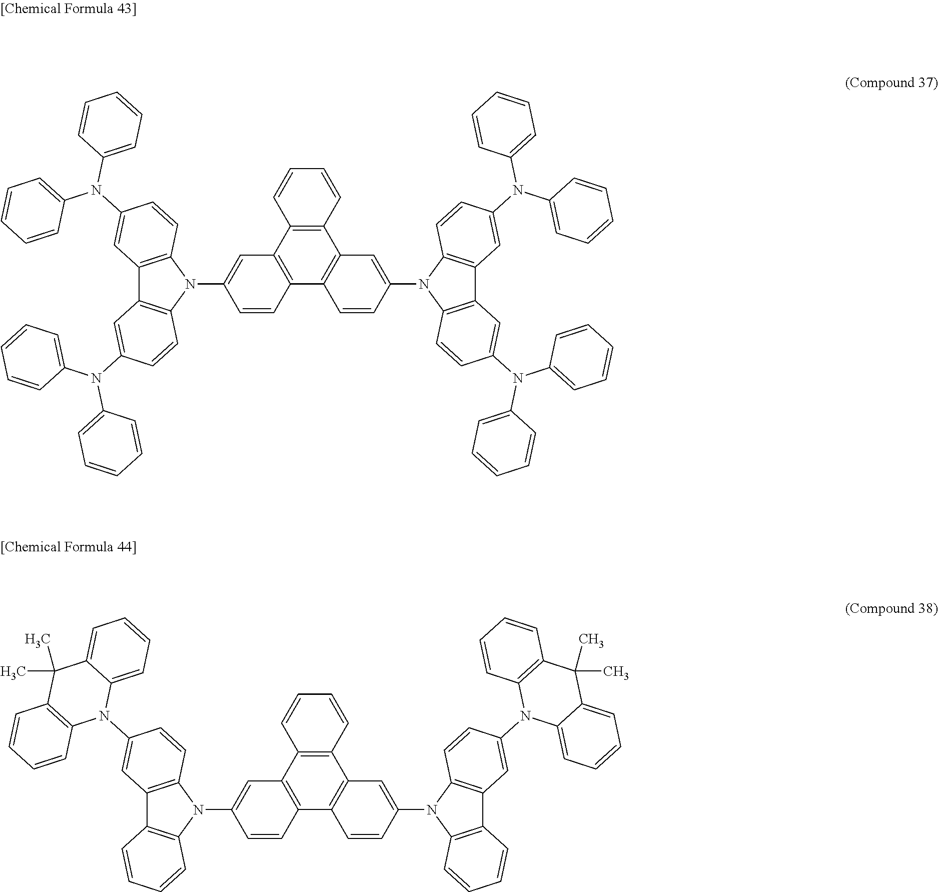

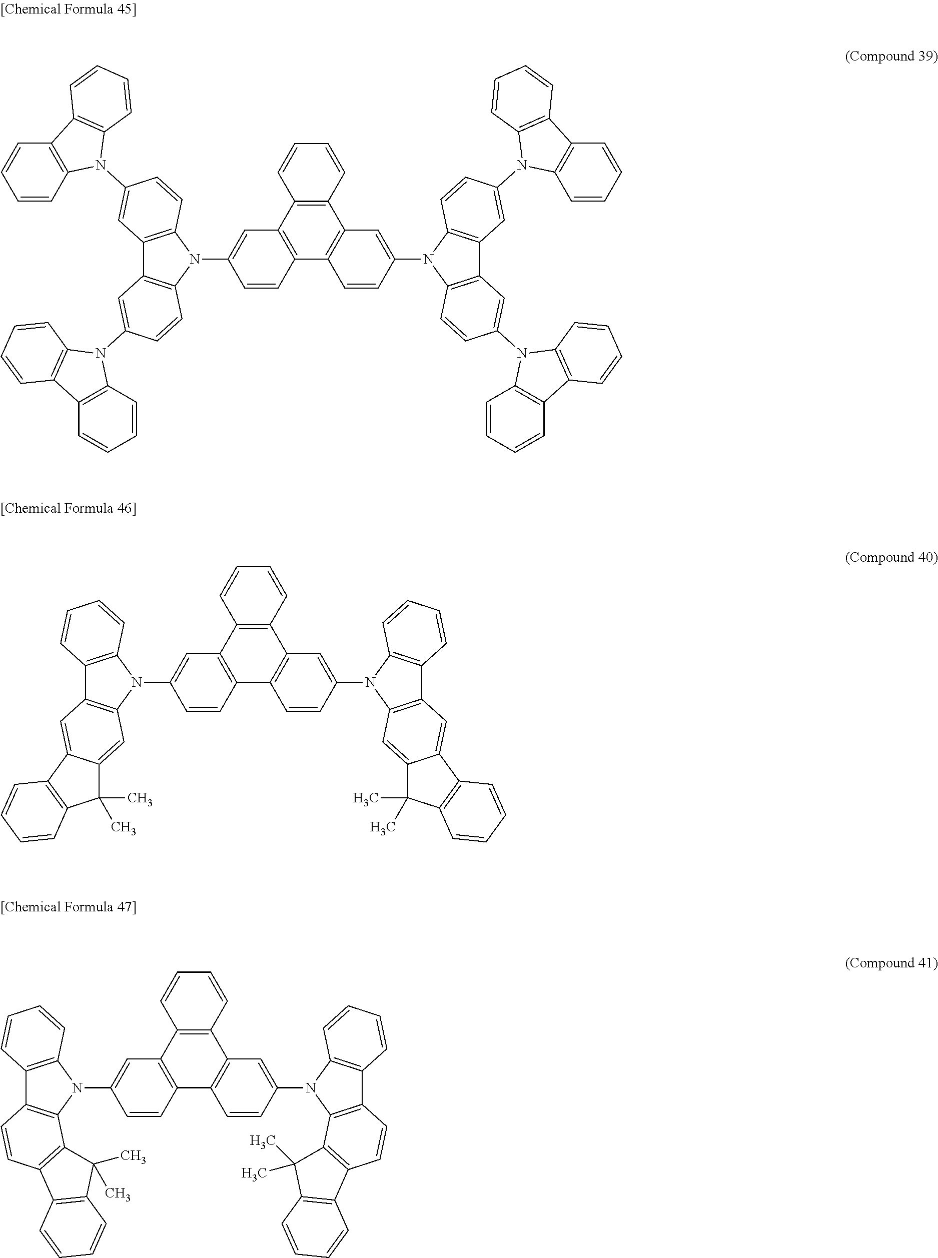

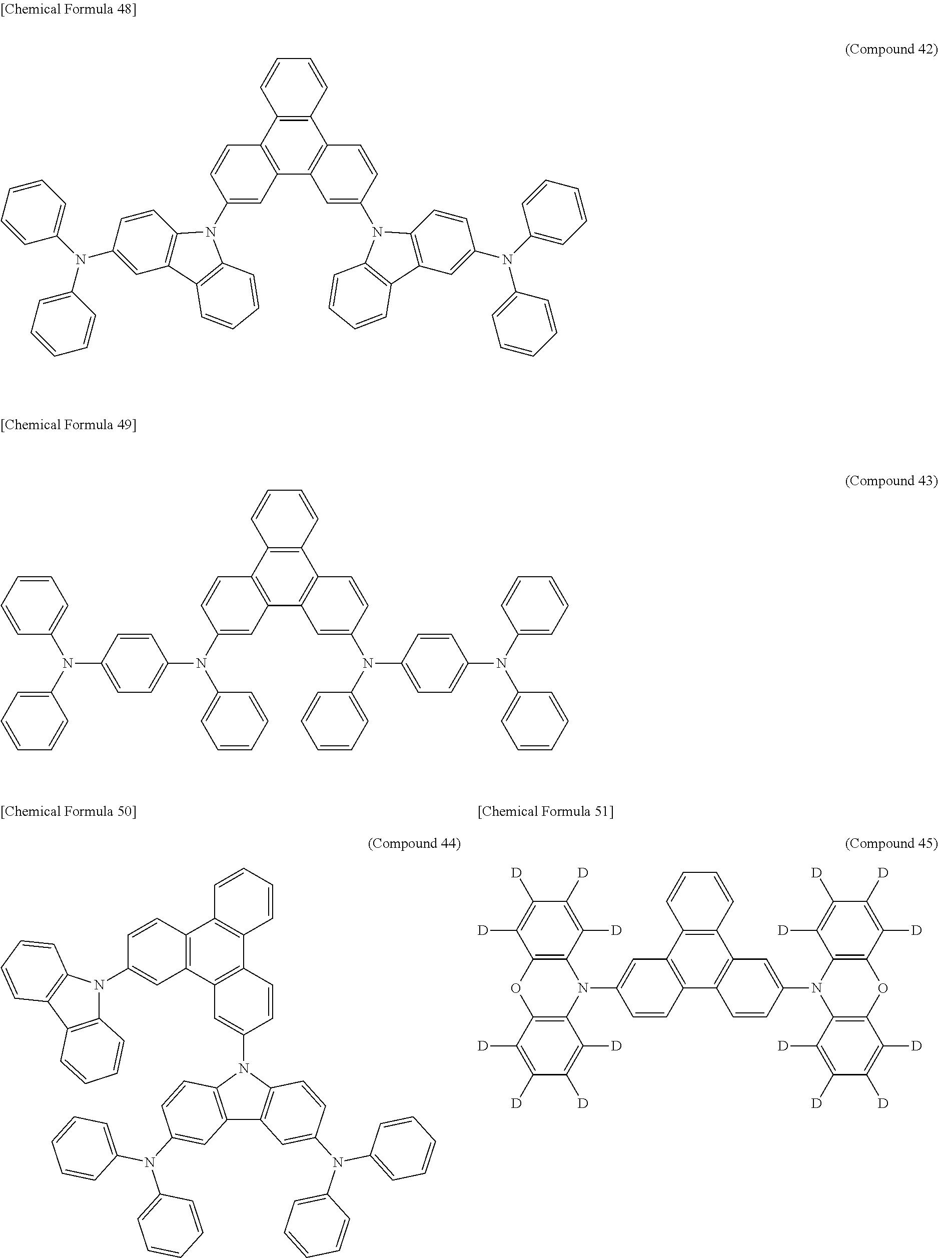

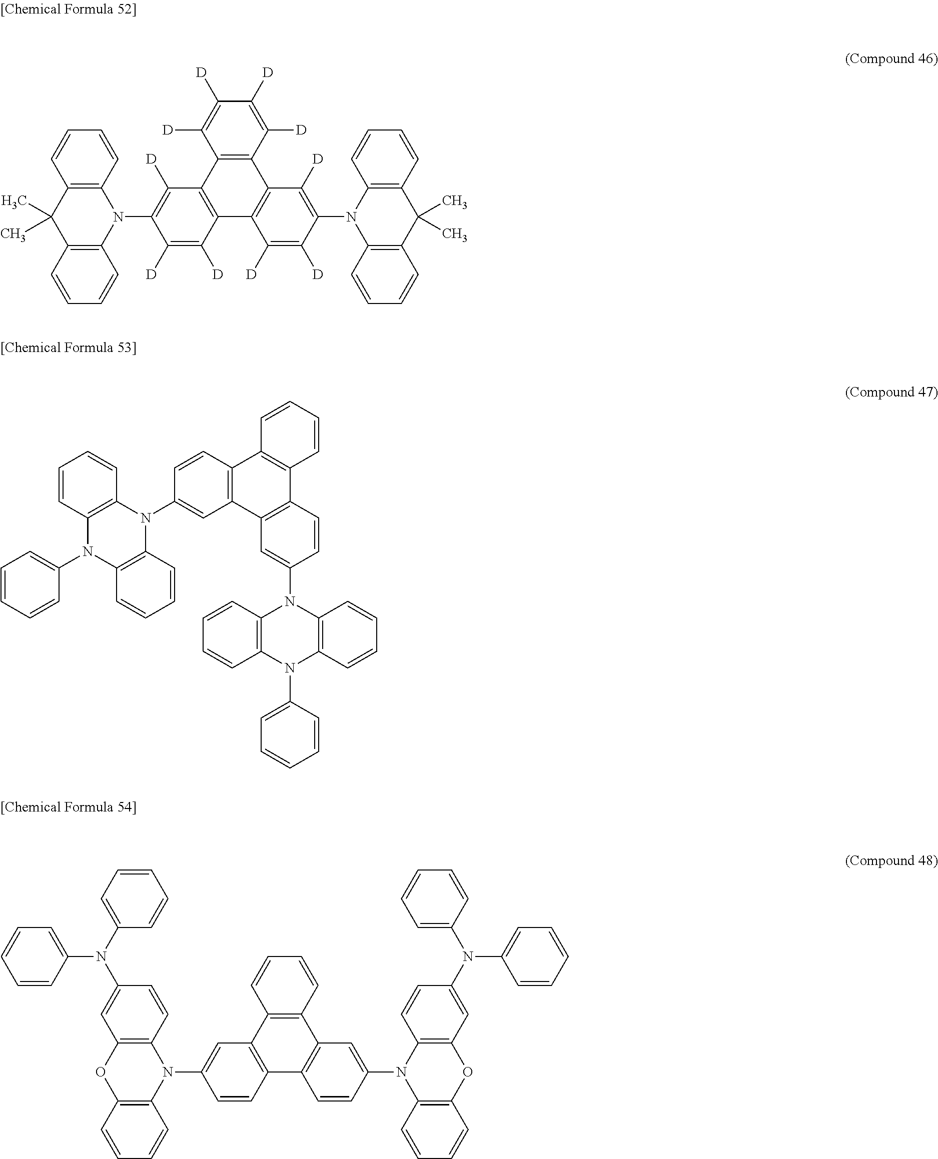

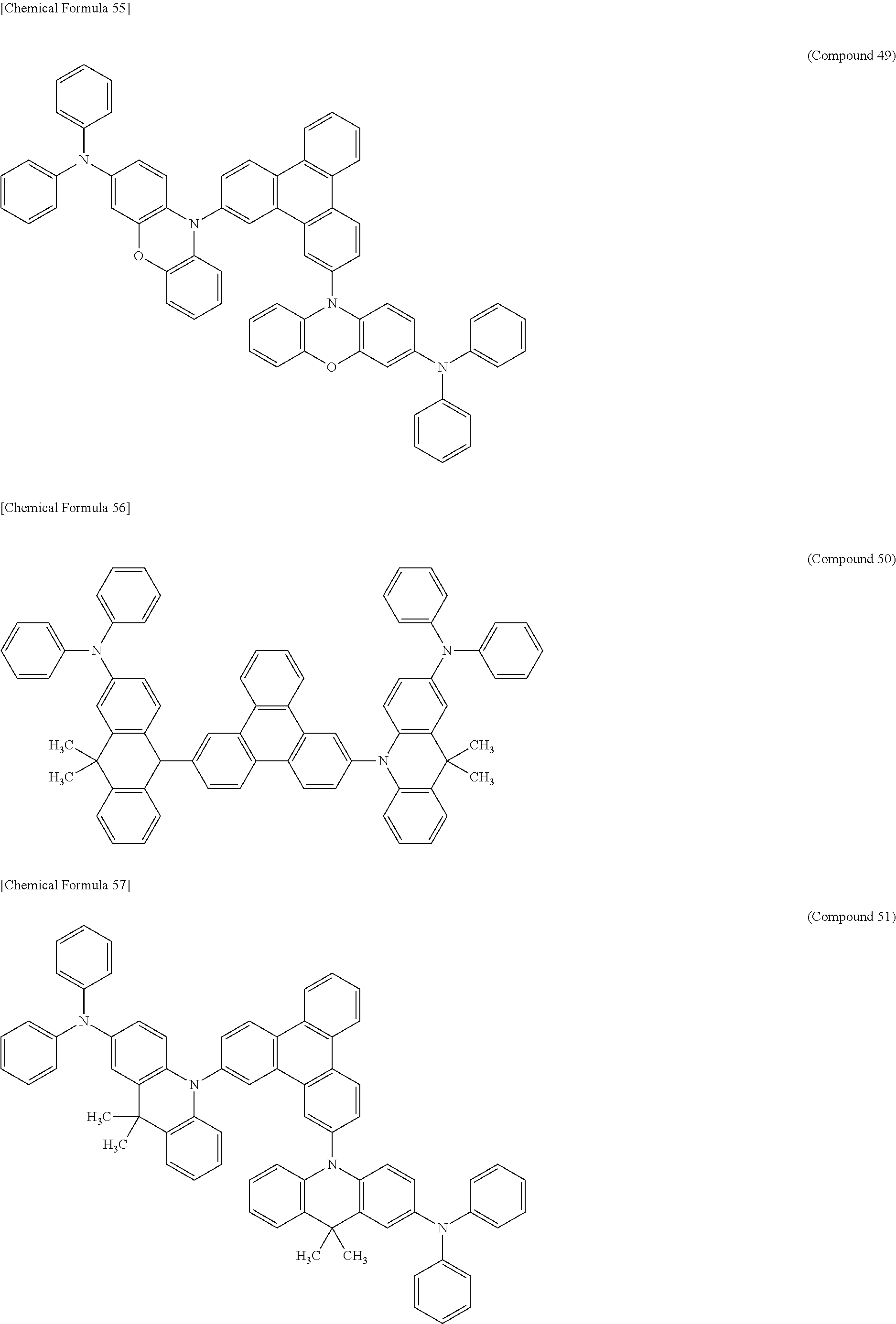

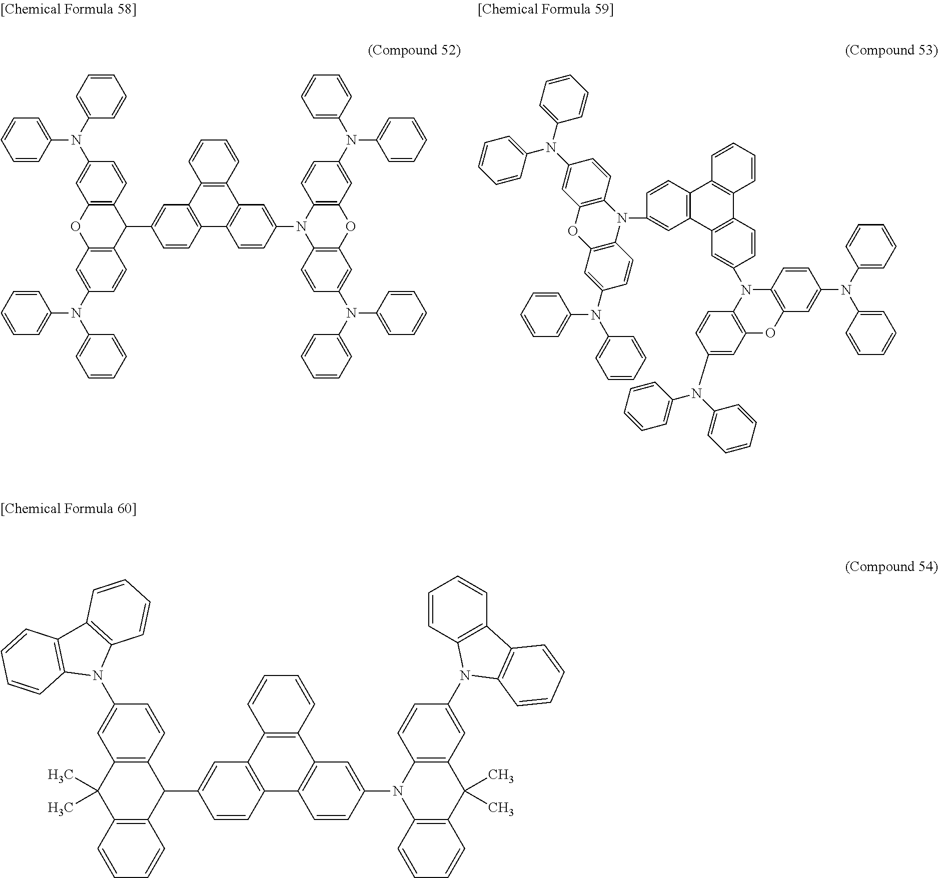

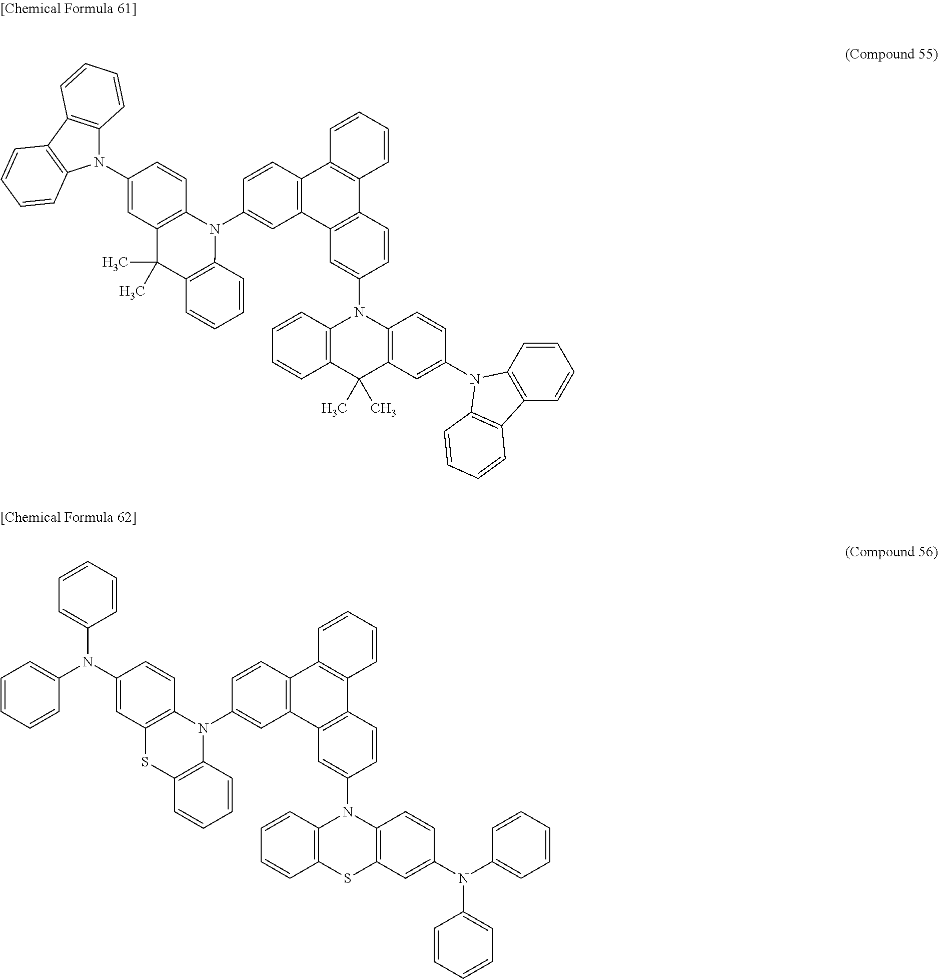

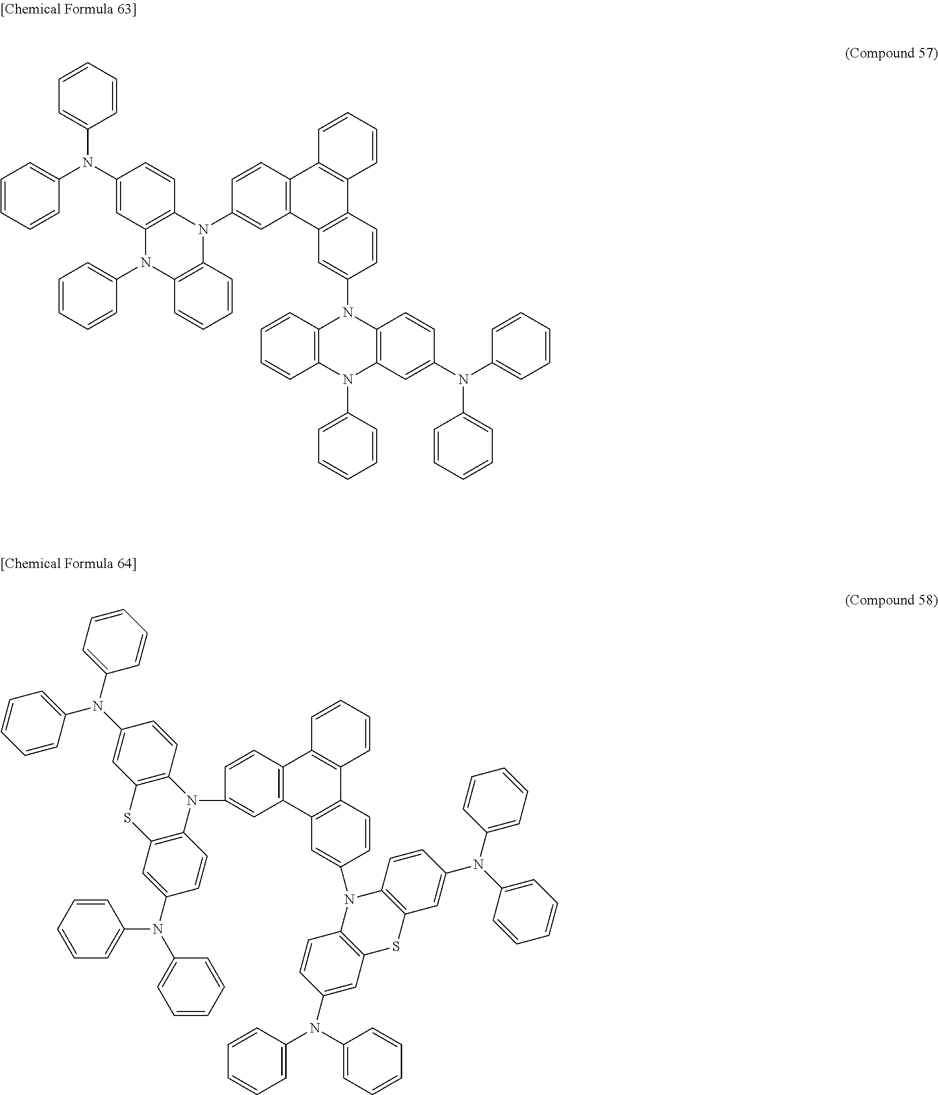

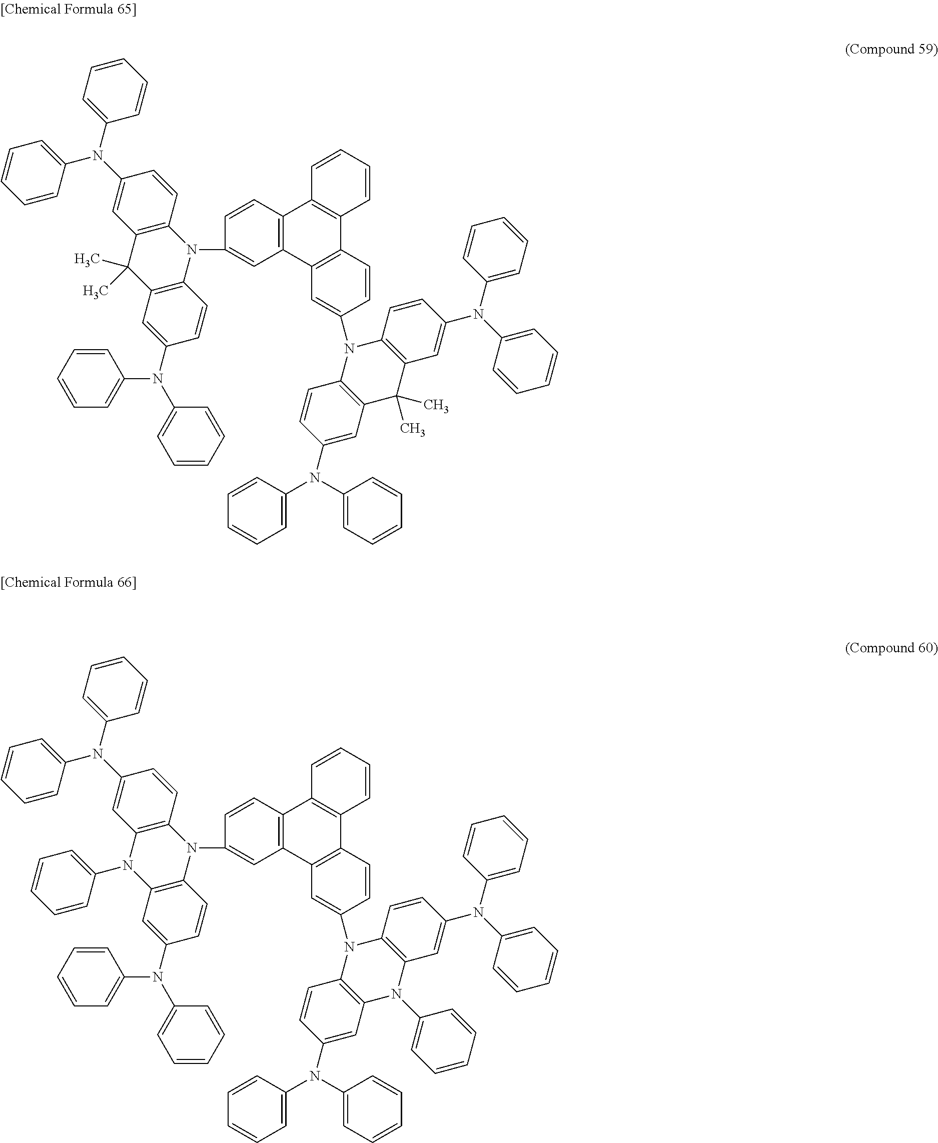

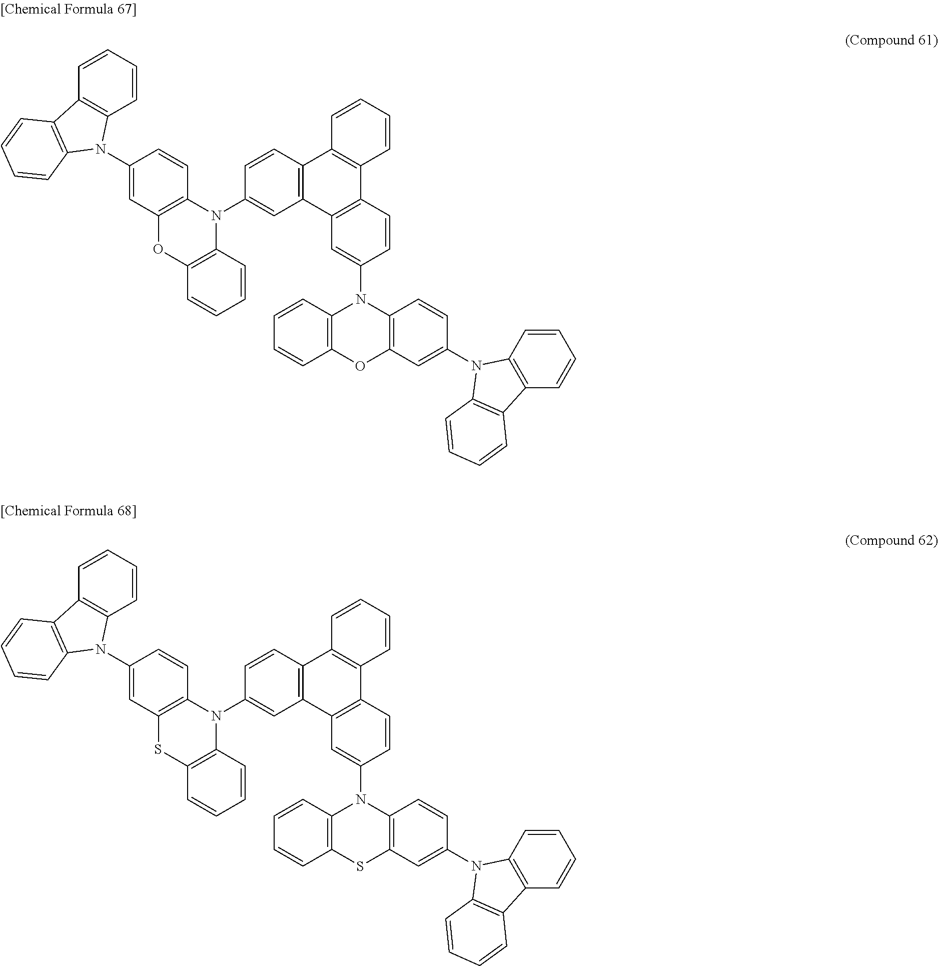

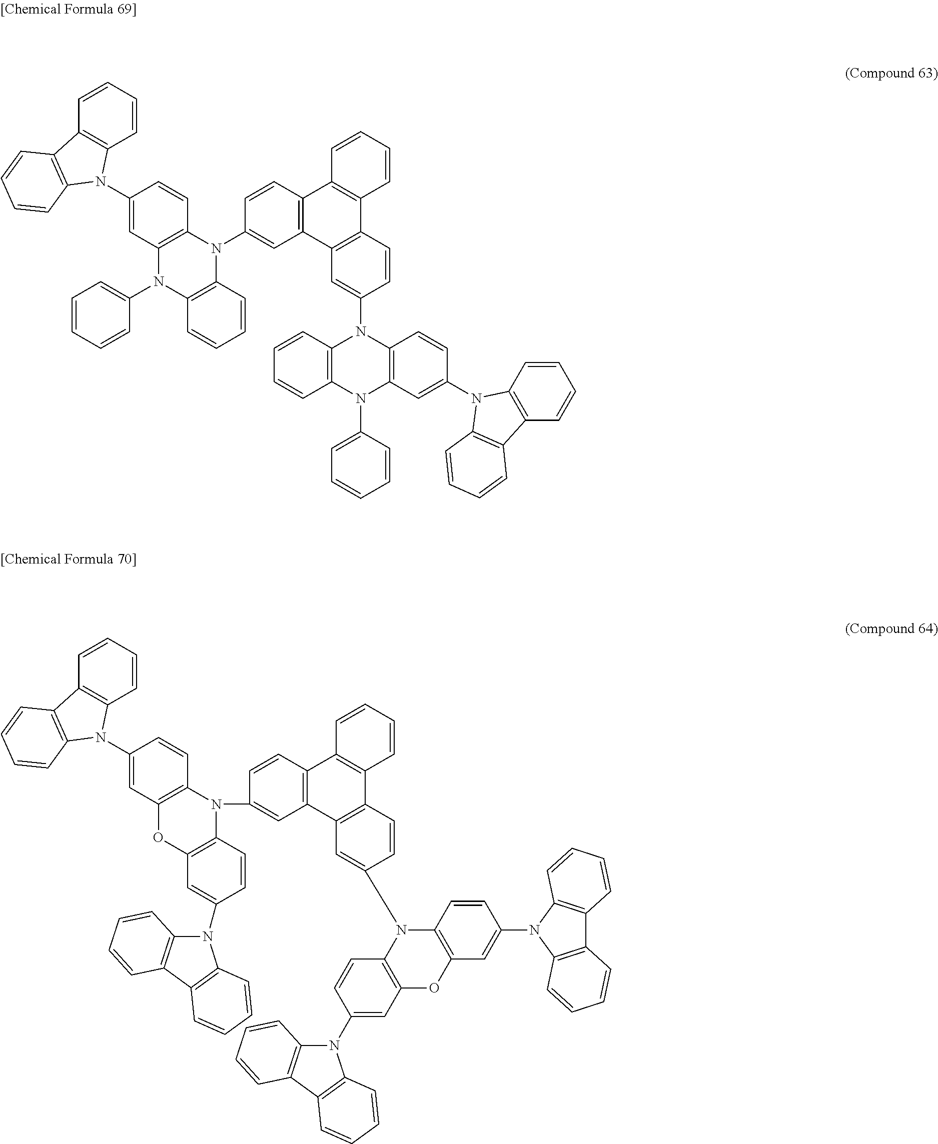

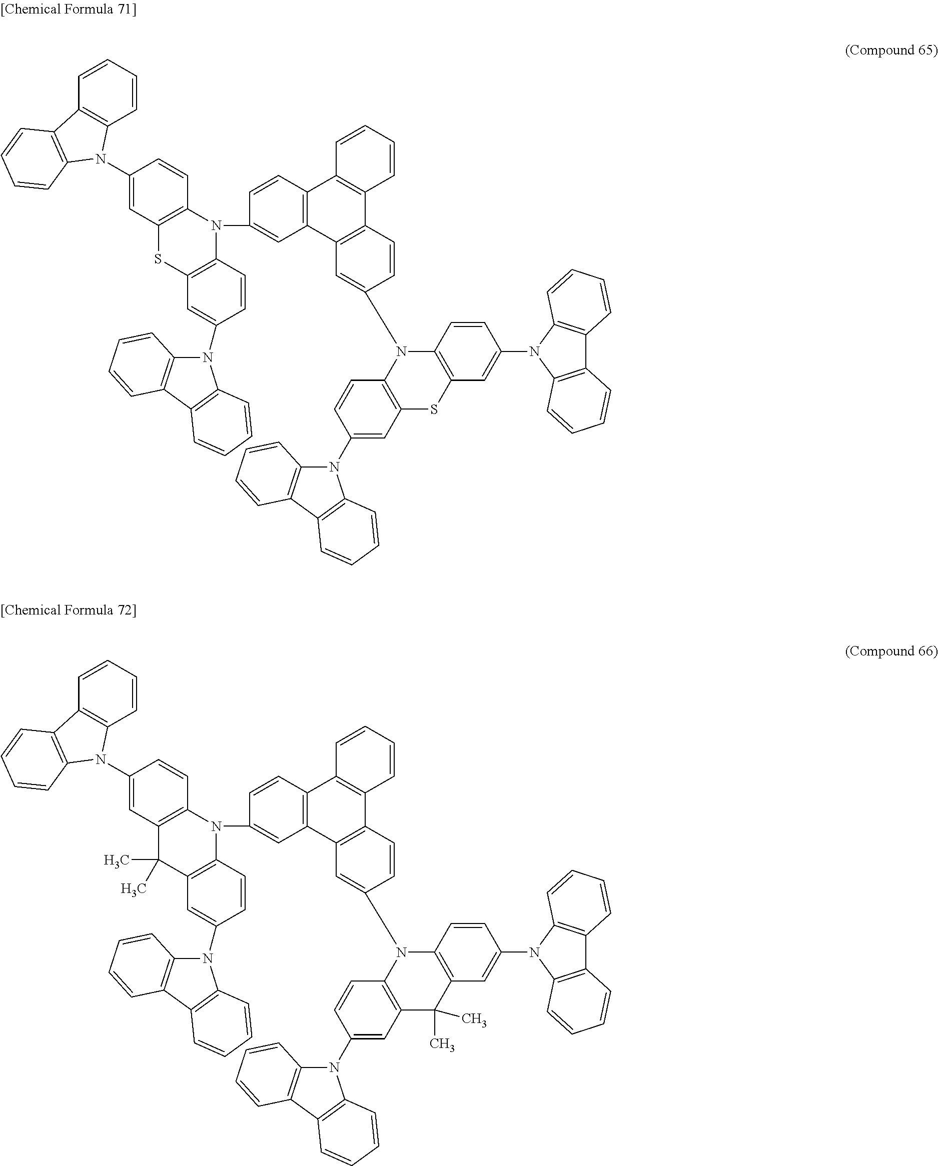

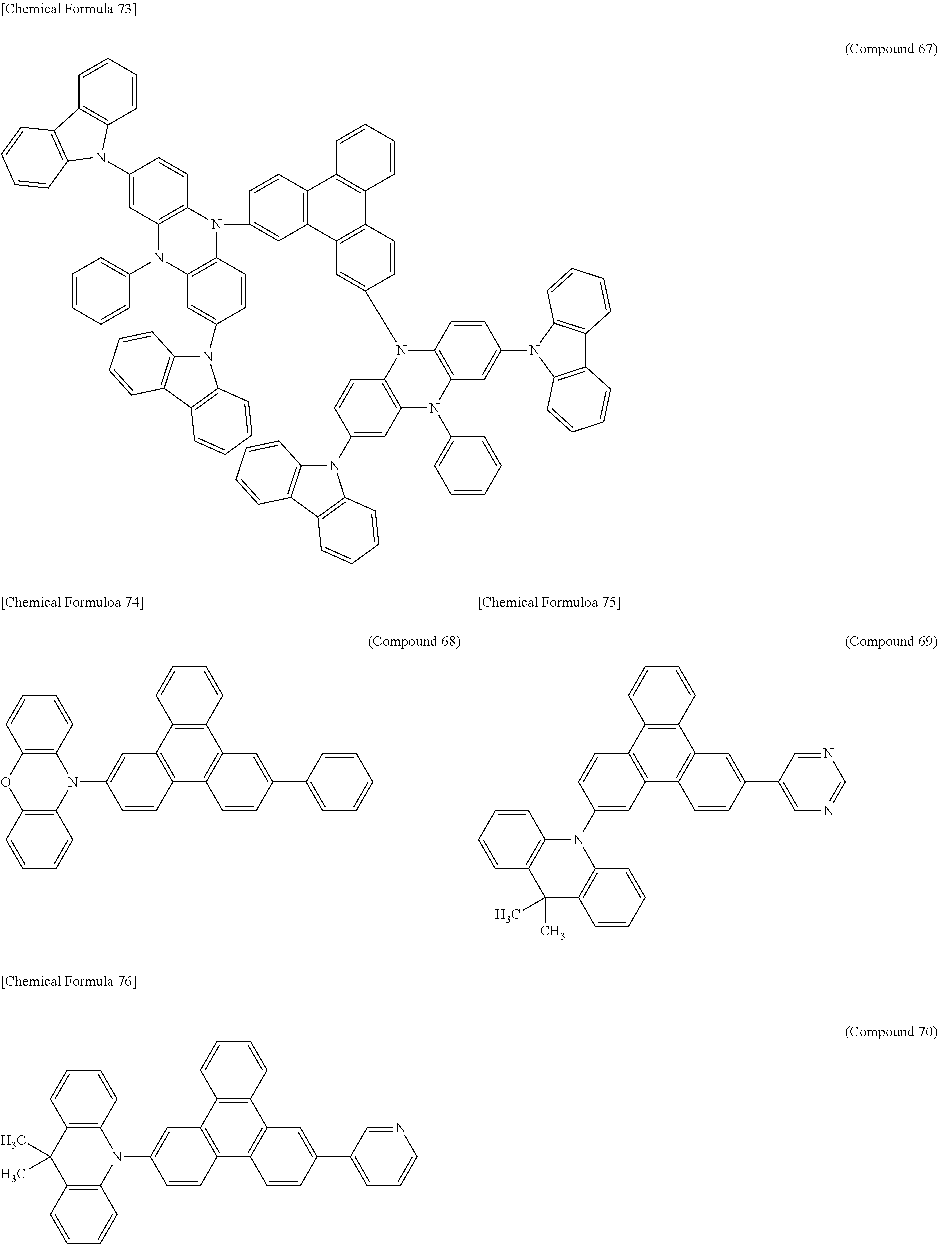

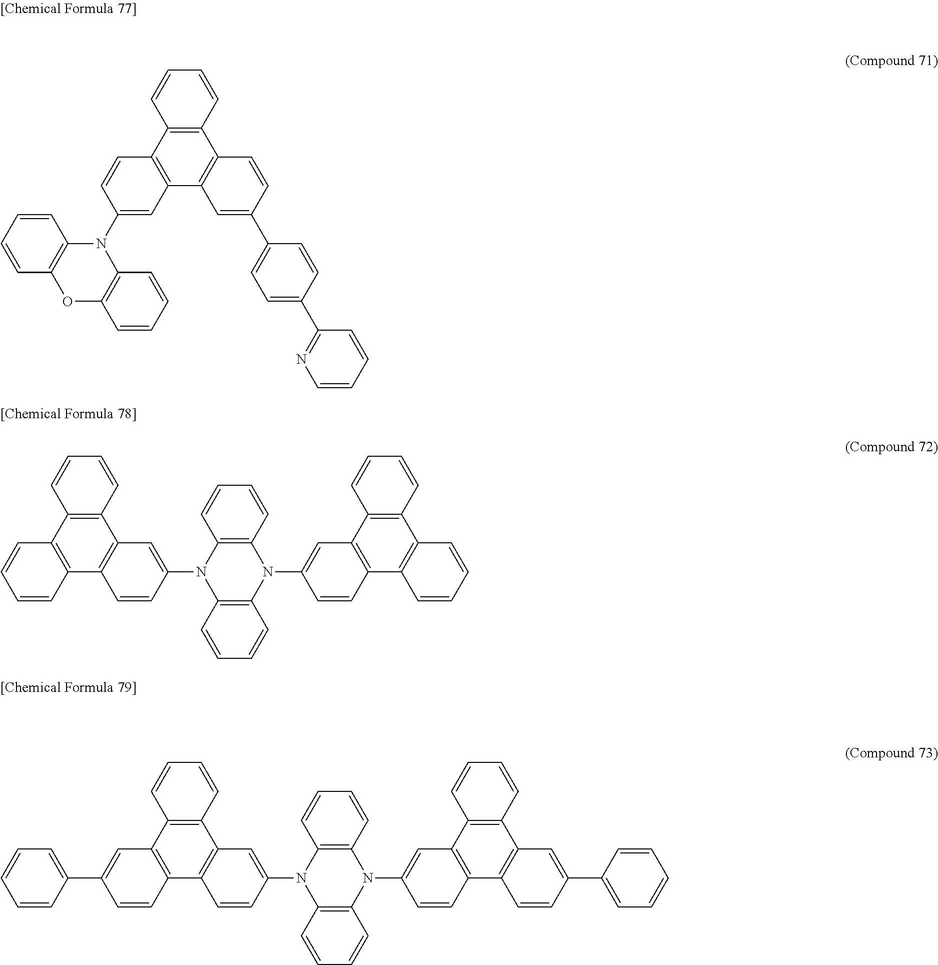

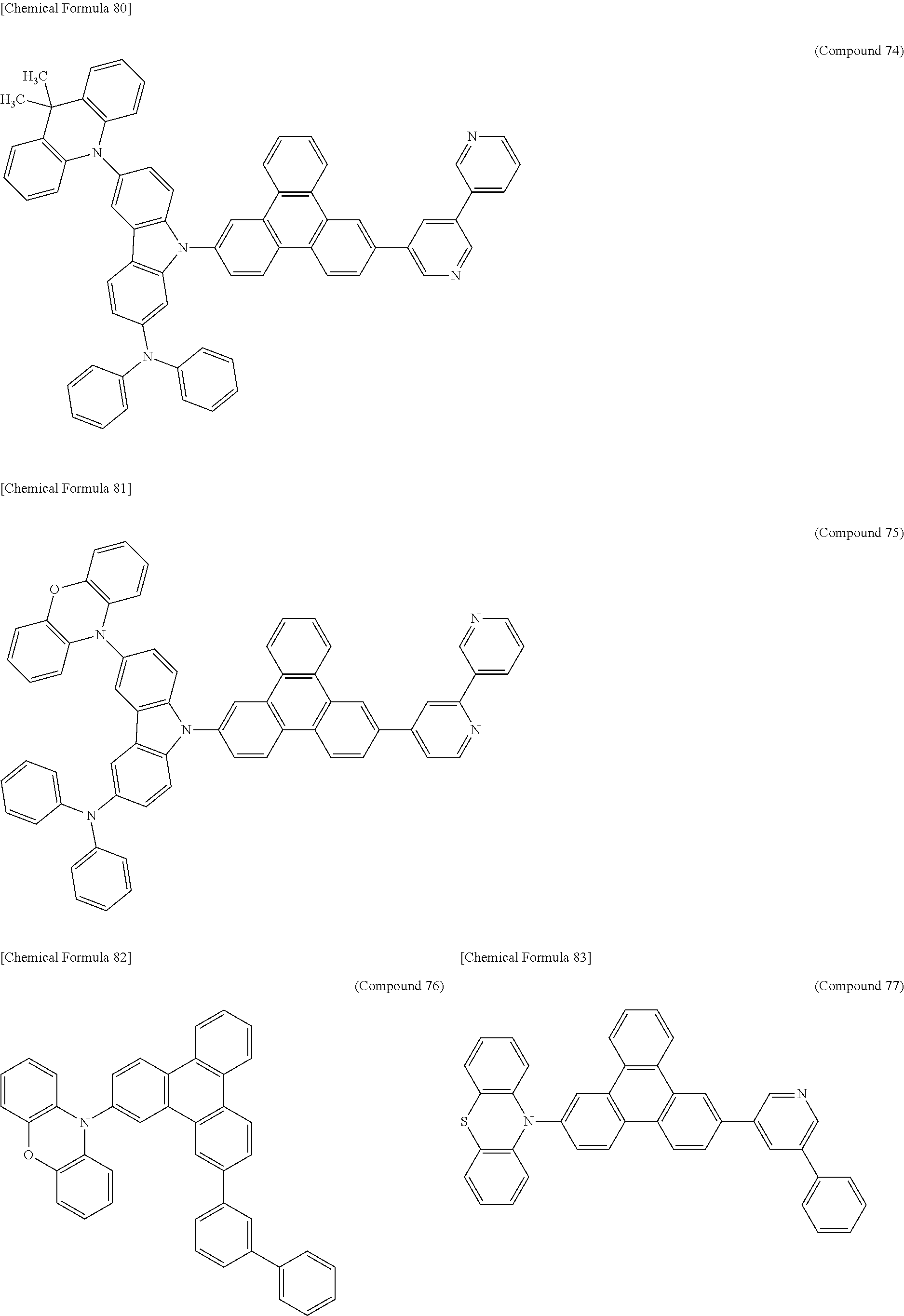

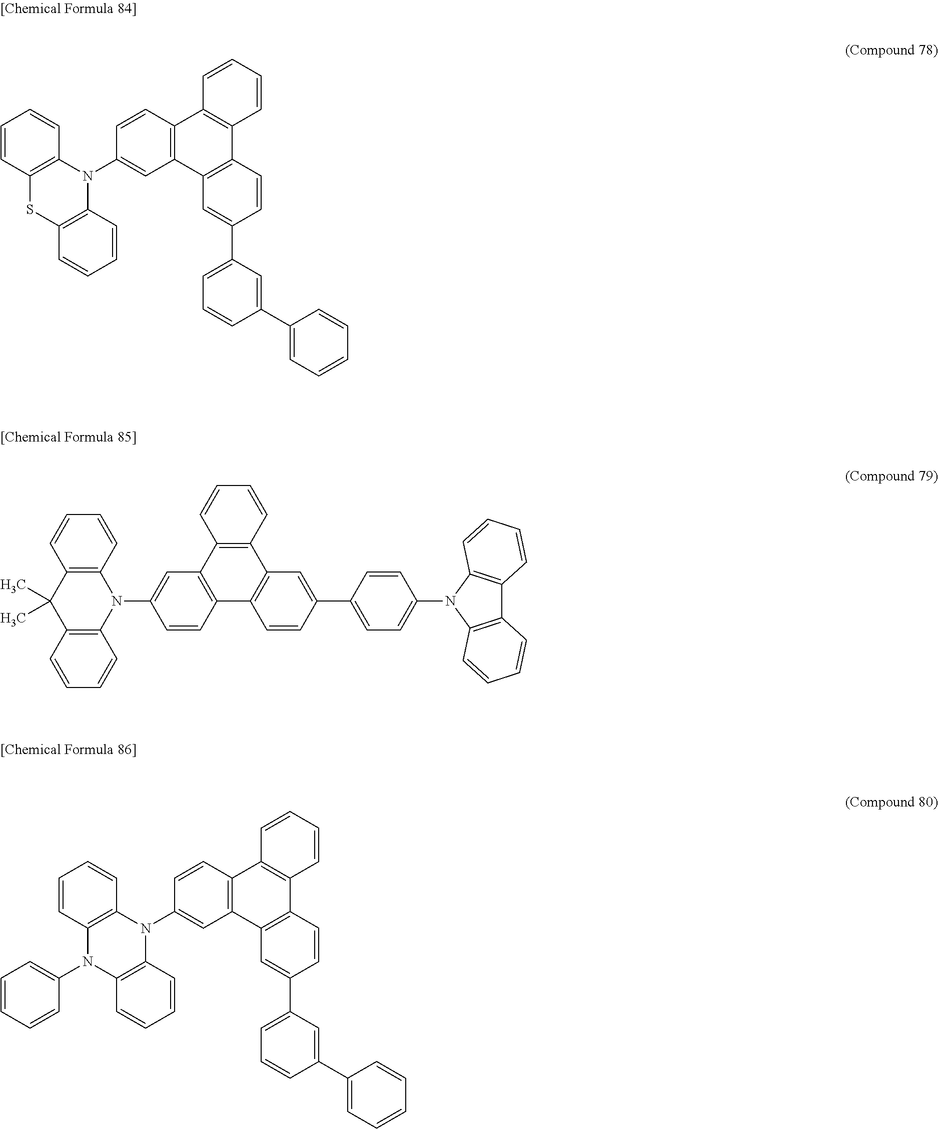

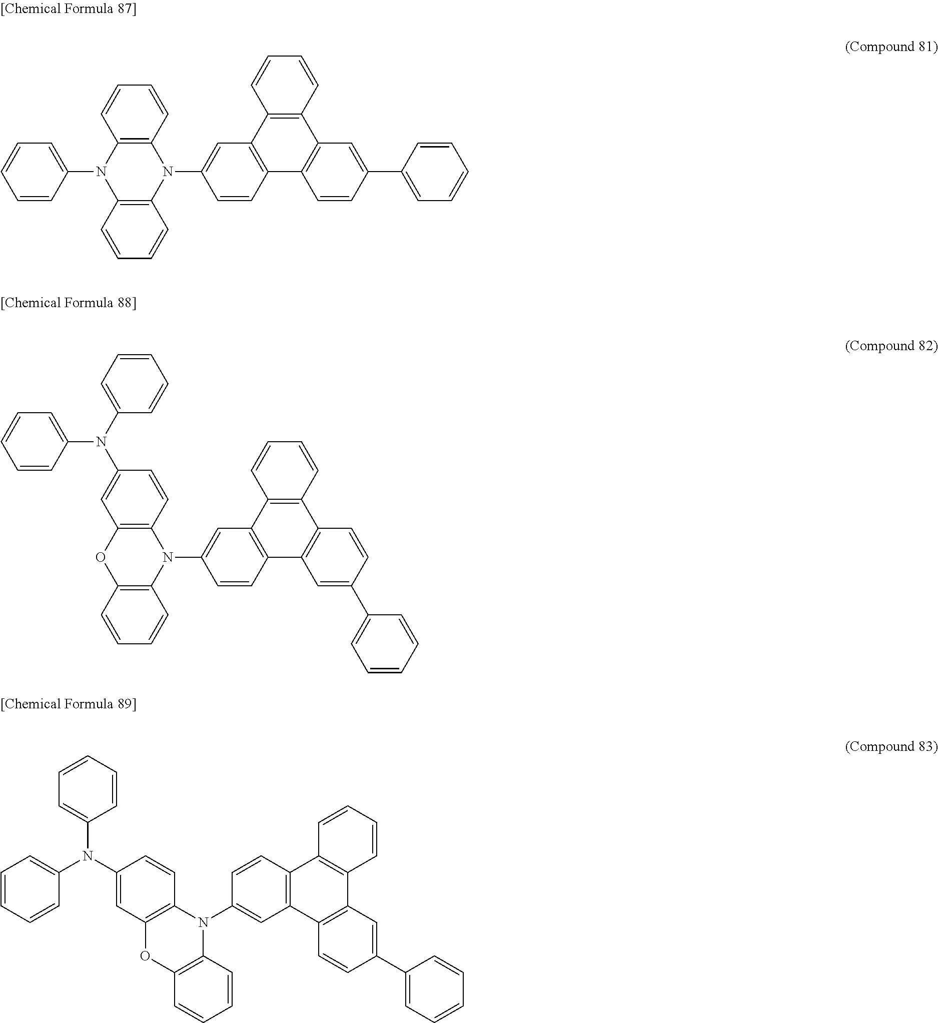

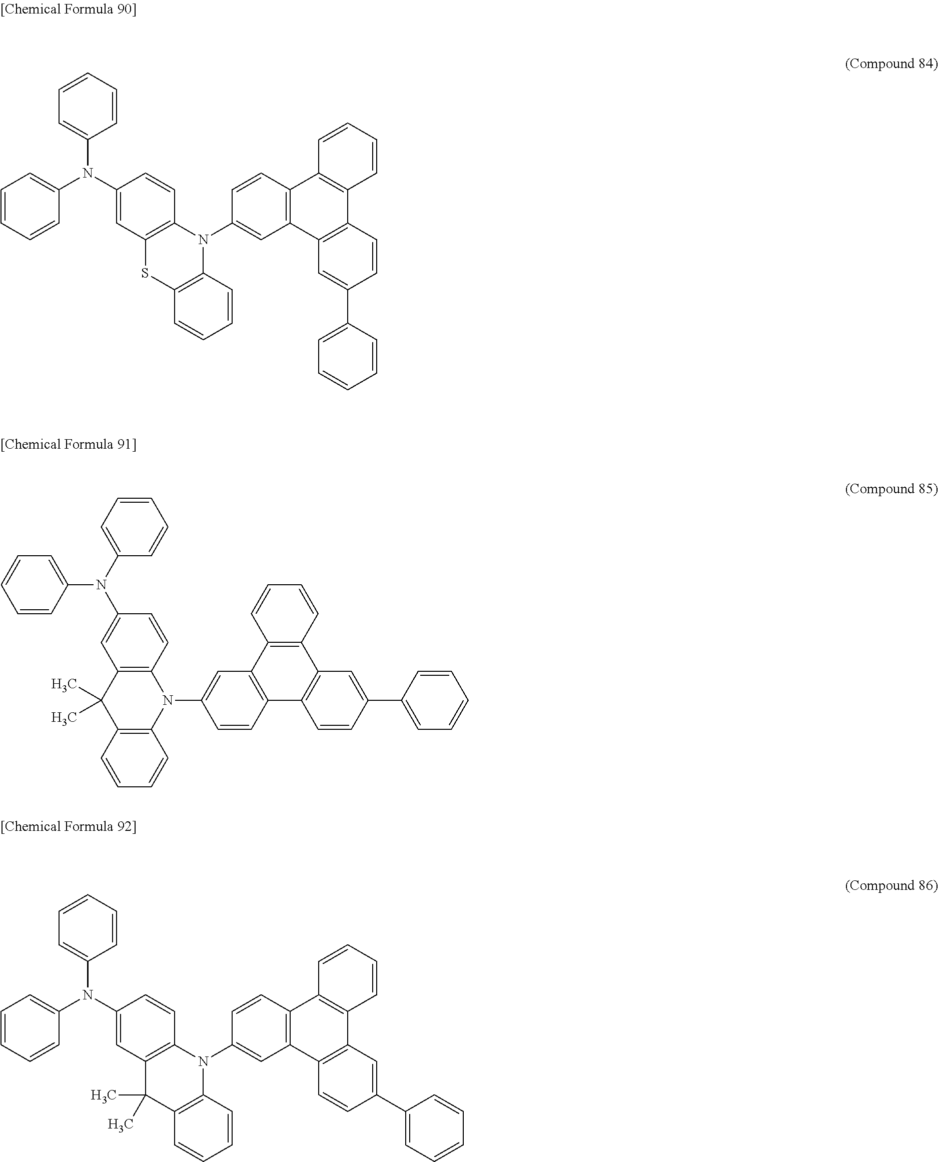

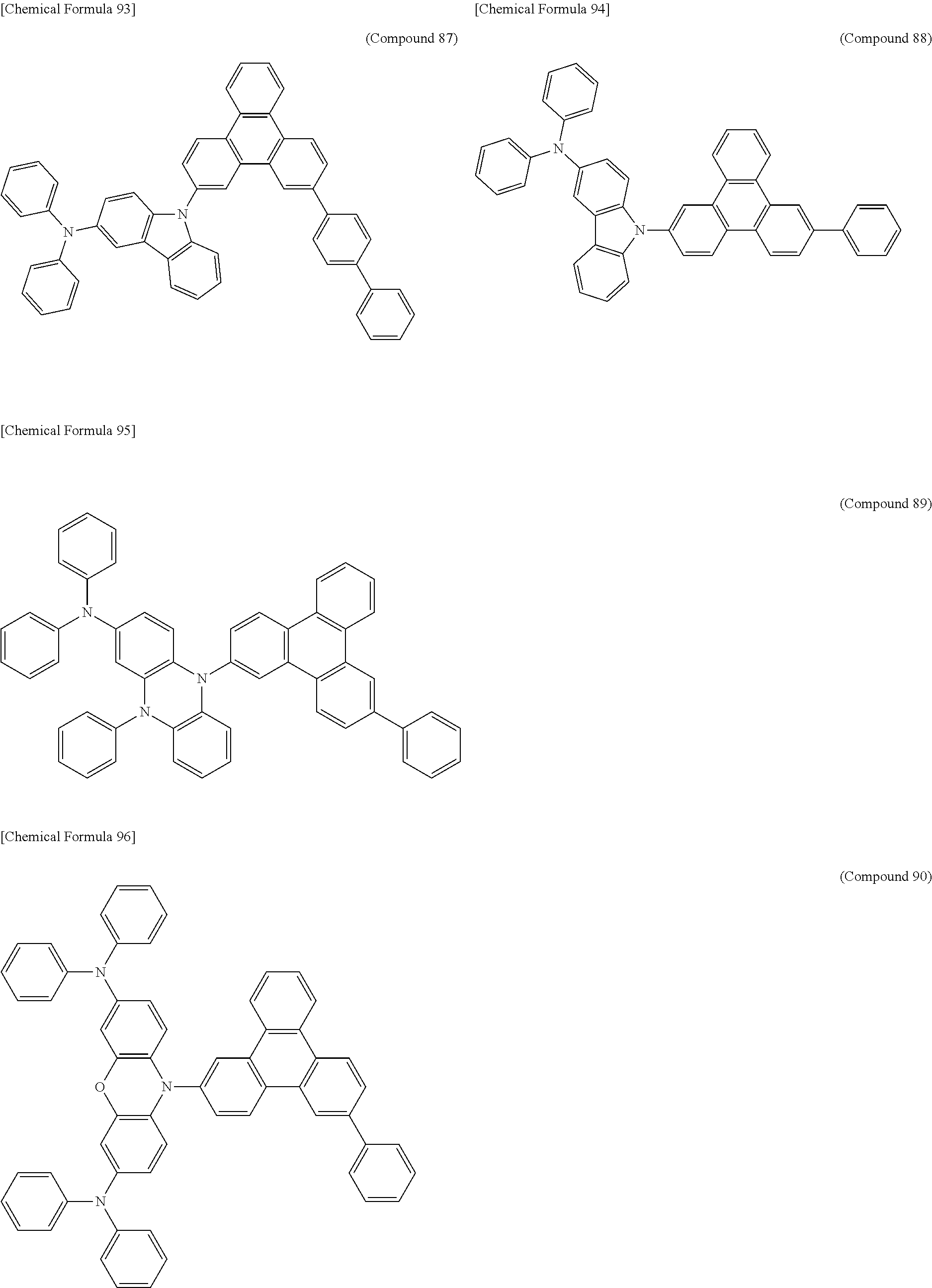

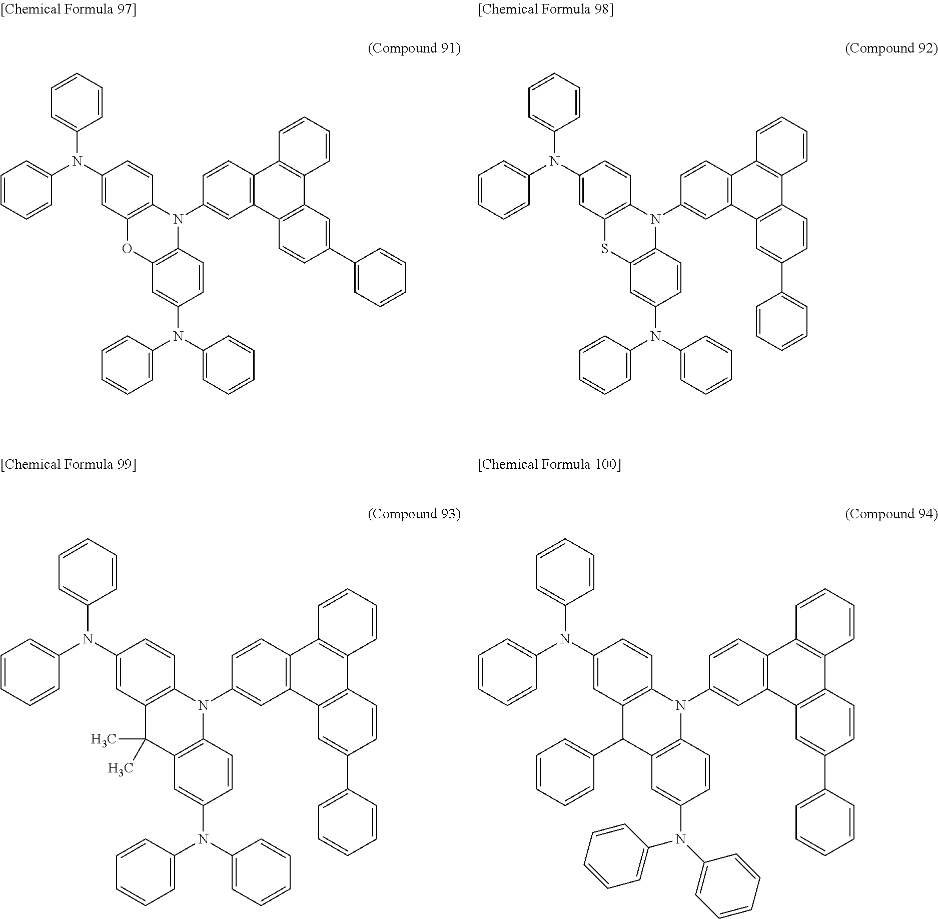

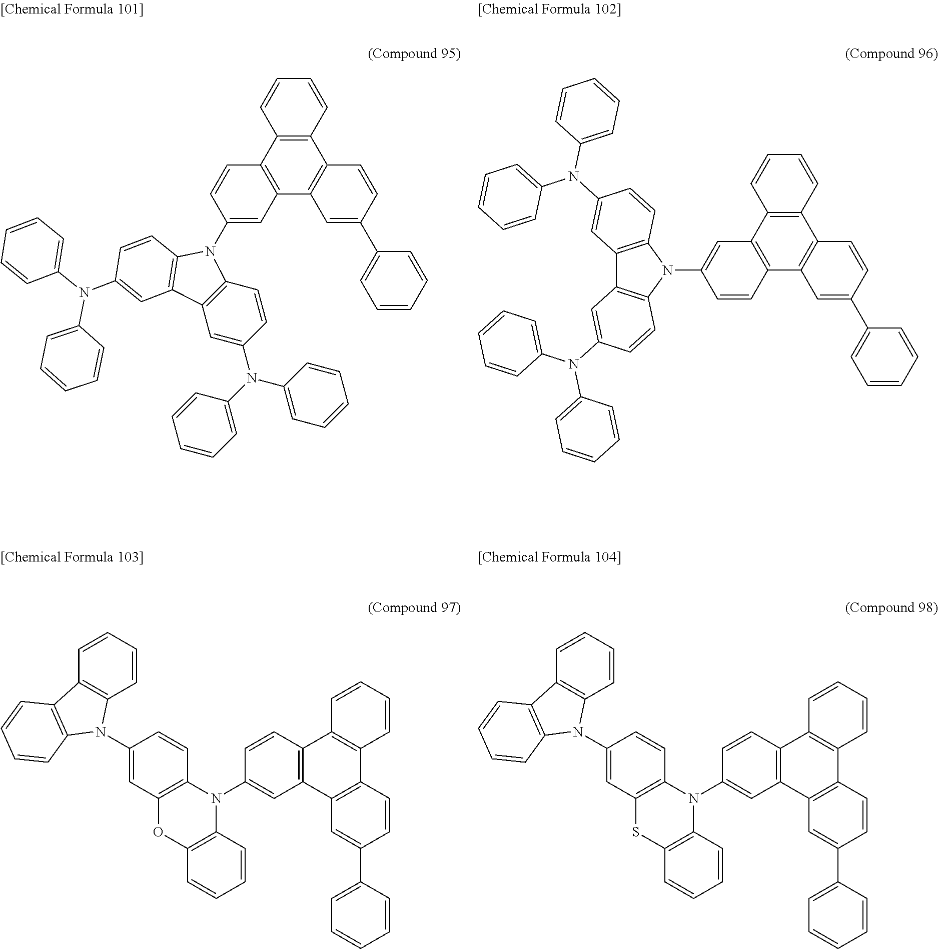

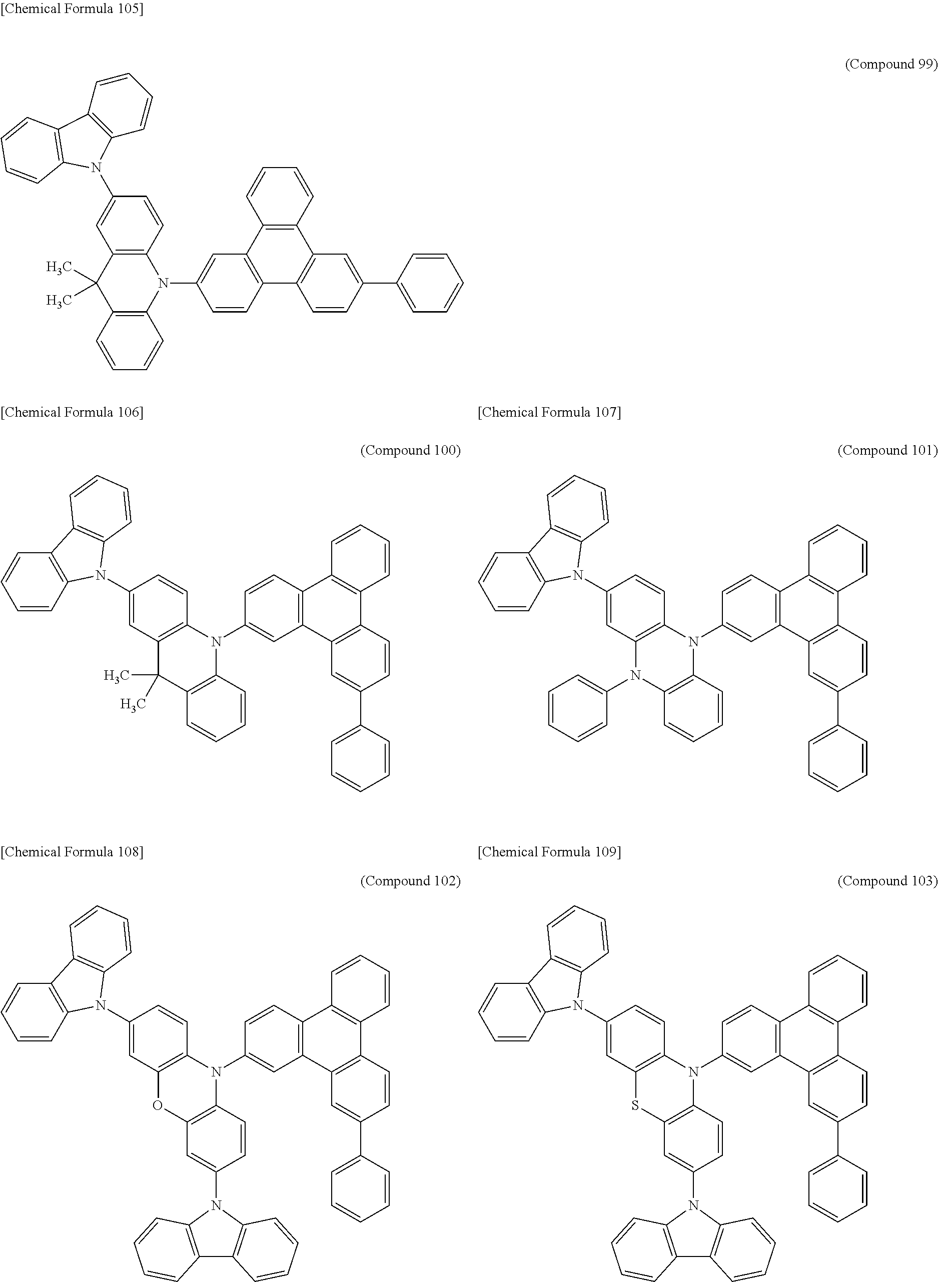

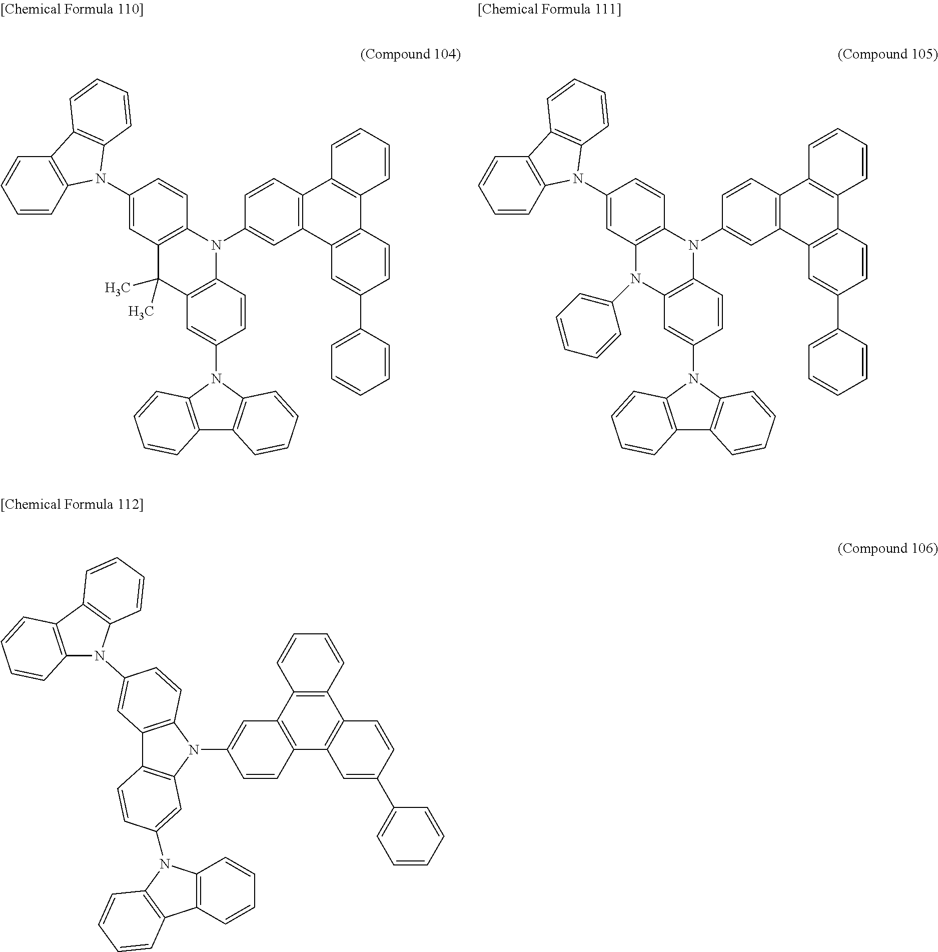

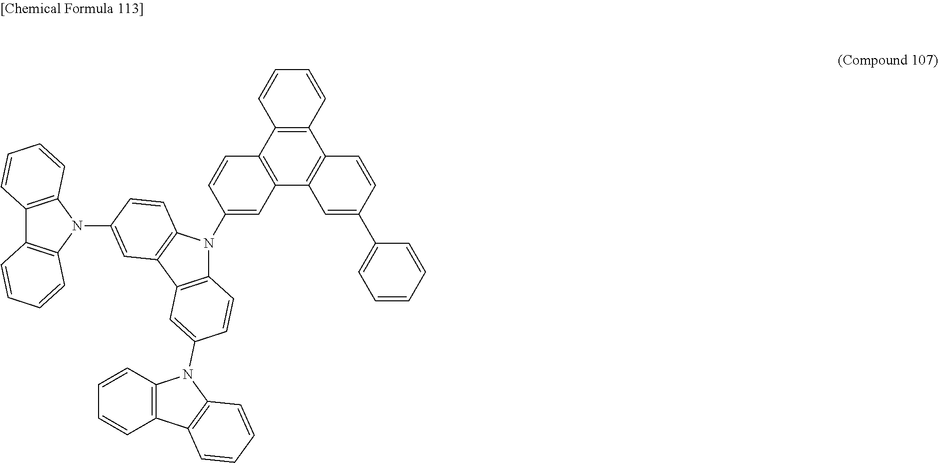

These compounds were purified by methods such as column chromatography; adsorption using, for example, a silica gel, activated carbon, or activated clay; recrystallization or crystallization using a solvent; and sublimation. The compounds were identified by an NMR analysis. A work function was measured as a material property value. The work function can be used as an index of energy level as a material for a light emitting layer.

For the measurement of the work function, a 100 nm-thick thin film was fabricated on an ITO substrate, and an atmosphere photoelectron spectrometer (AC-3 produced by Riken Keiki Co., Ltd.) was used.

The organic EL device of the present invention may have a structure including an anode, a hole injection layer, a hole transport layer, an electron blocking layer, a light emitting layer, a hole blocking layer, an electron transport layer, and a cathode successively formed on a substrate, optionally with an electron injection layer between the electron transport layer and the cathode. In such multilayer structures, some of the organic layers may be omitted. For example, the device may be configured to include an anode, a hole transport layer, a light emitting layer, an electron transport layer, an electron injection layer, and a cathode successively formed on a substrate, or to include an anode, a hole transport layer, a light emitting layer, an electron transport layer, and a cathode successively formed on a substrate.

Each of the light emitting layer, the hole transport layer, and the electron transport layer may have a laminate structure of two or more layers.

Electrode materials with high work functions such as ITO and gold are used as the anode of the organic EL device of the present invention. Examples of material used for the hole injection layer of the organic EL device of the present invention can be naphthalenediamine derivatives; starburst-type triphenylamine derivatives; triphenylamine trimers and tetramers such as an arylamine compound having a structure in which three or more triphenylamine structures are joined within a molecule via a single bond or a divalent group that does not contain a heteroatom; accepting heterocyclic compounds such as hexacyano azatriphenylene; and coating-type polymer materials, in addition to porphyrin compounds as represented by copper phthalocyanine. These materials may be formed into a thin film by a vapor deposition method or other known methods such as a spin coating method and an inkjet method.

Examples of material used for the hole transport layer of the organic EL device of the present invention can be benzidine derivatives such as N,N′-diphenyl-N,N′-di(m-tolyl)-benzidine (hereinafter referred to as TPD), N,N′-diphenyl-N,N′-di(α-naphthyl)-benzidine (hereinafter referred to as NPD), and N,N,N′,N′-tetrabiphenylylbenzidine; 1,1-bis[(di-4-tolylamino)phenyl]cyclohexane (TAPC); various triphenylamine trimers and tetramers; and carbazole derivatives, in addition to compounds containing an m-carbazolylphenyl group. These may be individually deposited for film forming, may be used as a single layer deposited mixed with other materials, or may be formed as a laminate of individually deposited layers, a laminate of mixedly deposited layers, or a laminate of the individually deposited layer and the mixedly deposited layer. Examples of material used for the hole injection/transport layer can be coating-type polymer materials such as poly(3,4-ethylenedioxythiophene) (PEDOT)/poly(styrene sulfonate) (PSS). These materials may be formed into a thin-film by a vapor deposition method or other known methods such as a spin coating method and an inkjet method.

Material used for the hole injection layer or the hole transport layer may be obtained by p-doping trisbromophenylamine hexachloroantimony into the material commonly used for these layers, or may be, for example, polymer compounds each having, as a part of the compound structure, a structure of a benzidine derivative such as TPD.

Examples of material used for the electron blocking layer of the organic EL device of the present invention can be compounds having an electron blocking effect, including carbazole derivatives such as 4,4′,4″-tri(N-carbazolyl)triphenylamine (hereinafter referred to as TCTA), 9,9-bis[4-(carbazol-9-yl)phenyl]fluorene, 1,3-bis(carbazol-9-yl)benzene (hereinafter referred to as mCP), and 2,2-bis(4-carbazol-9-ylphenyl)adamantane (Ad-Cz); and compounds having a triphenylsilyl group and a triarylamine structure, as represented by 9-[4-(carbazol-9-yl)phenyl]-9-[4-(triphenylsilyl)phenyl]-9H-fluorene. These may be individually deposited for film forming, may be used as a single layer deposited mixed with other materials, or may be formed as a laminate of individually deposited layers, a laminate of mixedly deposited layers, or a laminate of the individually deposited layer and the mixedly deposited layer. These materials may be formed into a thin film by a vapor deposition method or other known methods such as a spin coating method and an inkjet method.

Examples of material used for the light emitting layer of the organic EL device of the present invention can be the compounds of the general formula (1) having a triphenylene ring structure of the present invention; delayed fluorescence-emitting materials such as CDCB derivatives of PIC-TRZ (refer to Non-Patent Document 1, for example), CC2TA (refer to Non-Patent Document 3, for example), PXZ-TRZ (refer to Non-Patent Document 4, for example), 4CzIPN or the like (refer to Non-Patent Document 5, for example); various metal complexes including, for example, quinolinol derivative metal complexes such as tris(8-hydroxyquinoline)aluminum (hereinafter referred to as Alg₃); anthracene derivatives; bis(styryl)benzene derivatives; pyrene derivatives; oxazole derivatives; and polyparaphenylene vinylene derivatives. Further, the light emitting layer may be made of a host material and a dopant material. In this case, examples of the host material can be mCP, thiazole derivatives, benzimidazole derivatives, and polydialkyl fluorene derivatives. Examples of the dopant material can be the compounds of the general formula (1) having a triphenylene ring structure of the present invention; delayed fluorescence-emitting materials such as CDCB derivatives of PIC-TRZ, CC2TA, PXZ-TRZ, 4CzIPN or the like; quinacridone, coumarin, rubrene, anthracene, perylene, and derivatives thereof; benzopyran derivatives; rhodamine derivatives; and aminostyryl derivatives. These may be individually deposited for film forming, may be used as a single layer deposited mixed with other materials, or may be formed as a laminate of individually deposited layers, a laminate of mixedly deposited layers, or a laminate of the individually deposited layer and the mixedly deposited layer.

Further, the light-emitting material may be phosphorescent light-emitting material. Phosphorescent materials as metal complexes of metals such as iridium and platinum may be used as the phosphorescent light-emitting material. Examples of the phosphorescent materials include green phosphorescent materials such as Ir(ppy)₃, blue phosphorescent materials such as FIrpic and FIr6, and red phosphorescent materials such as Btp₂Ir(acac) and Ir(piq)₃. Here, carbazole derivatives such as 4,4′-di(N-carbazolyl)biphenyl (hereinafter referred to as CBP), TCTA, and mCP may be used as the hole injecting and transporting host material. Compounds such as p-bis(triphenylsilyl)benzene (UGH2), and 2,2′,2″-(1,3,5-phenylene)-tris(1-phenyl-1H-benzimidazole) (hereinafter referred to as TPBI) may be used as the electron transporting host material. These may be individually deposited for film forming, may be used as a single layer deposited mixed with other materials, or may be formed as a laminate of individually deposited layers, a laminate of mixedly deposited layers, or a laminate of the individually deposited layer and the mixedly deposited layer.

In order to avoid concentration quenching, the doping of the host material with the phosphorescent light-emitting material should preferably be made by co-evaporation in a range of 1 to 30 weight percent with respect to the whole light emitting layer.

These materials may be formed into a thin-film by using a vapor deposition method or other known methods such as a spin coating method and an inkjet method.

It is also possible to produce a device of a structure that includes a light emitting layer produced with the compound of the present invention, and an adjacently laminated light emitting layer produced by using a compound of a different work function as the host material (refer to Non-Patent Document 6, for example).

The hole blocking layer of the organic EL device of the present invention may be formed by using hole blocking compounds such as various rare earth complexes, oxazole derivatives, triazole derivatives, and triazine derivatives, in addition to the compounds of the general formula (1) having a triphenylene ring structure of the present invention, the metal complexes of phenanthroline derivatives such as bathocuproin (BCP), and the metal complexes of quinolinol derivatives such as aluminum(III) bis(2-methyl-8-quinolinate)-4-phenylphenolate (hereinafter referred to as BAlq). These materials may also serve as the material of the electron transport layer. These may be individually deposited for film forming, may be used as a single layer deposited mixed with other materials, or may be formed as a laminate of individually deposited layers, a laminate of mixedly deposited layers, or a laminate of the individually deposited layer and the mixedly deposited layer. These materials may be formed into a thin-film by using a vapor deposition method or other known methods such as a spin coating method and an inkjet method.

The electron transport layer of the organic EL device of the present invention may be formed by using various metal complexes, triazole derivatives, triazine derivatives, oxadiazole derivatives, thiadiazole derivatives, carbodiimide derivatives, quinoxaline derivatives, phenanthroline derivatives, silole derivatives, and benzimidazole derivatives such as TPBI, in addition to the compounds of the general formula (1) having a triphenylene ring structure of the present invention, and metal complexes of quinolinol derivatives such as Alq₃ and BAlq. These may be individually deposited for film forming, may be used as a single layer deposited mixed with other materials, or may be formed as a laminate of individually deposited layers, a laminate of mixedly deposited layers, or a laminate of the individually deposited layer and the mixedly deposited layer. These materials may be formed into a thin-film by using a vapor deposition method or other known methods such as a spin coating method and an inkjet method.

Examples of material used for the electron injection layer of the organic EL device of the present invention can be alkali metal salts such as lithium fluoride and cesium fluoride; alkaline earth metal salts such as magnesium fluoride; and metal oxides such as aluminum oxide. However, the electron injection layer may be omitted in the preferred selection of the electron transport layer and the cathode.

Material used for the electron injection layer or the electron transport layer may be obtained by N-doping metals such as cesium into the material commonly used for these layers.

The cathode of the organic EL device of the present invention may be made of an electrode material with a low work function such as aluminum, or an alloy of an electrode material with an even lower work function such as a magnesium-silver alloy, a magnesium-indium alloy, or an aluminum-magnesium alloy.

Specific examples of preferred materials that may be used in the organic EL device of the present invention are shown below, but the materials that may be used in the present invention are not construed as being limited to the following exemplified compounds. The compound that is shown as a material having a particular function may also be used as a material having another function. In the structural formulae of the following exemplified compounds, R and R₂ to R₇ each independently represent a hydrogen atom or a substituent, and n represents an integer of 3 to 5.

Preferred examples of a compound that may also be used as the host material of the light emitting layer are shown below.

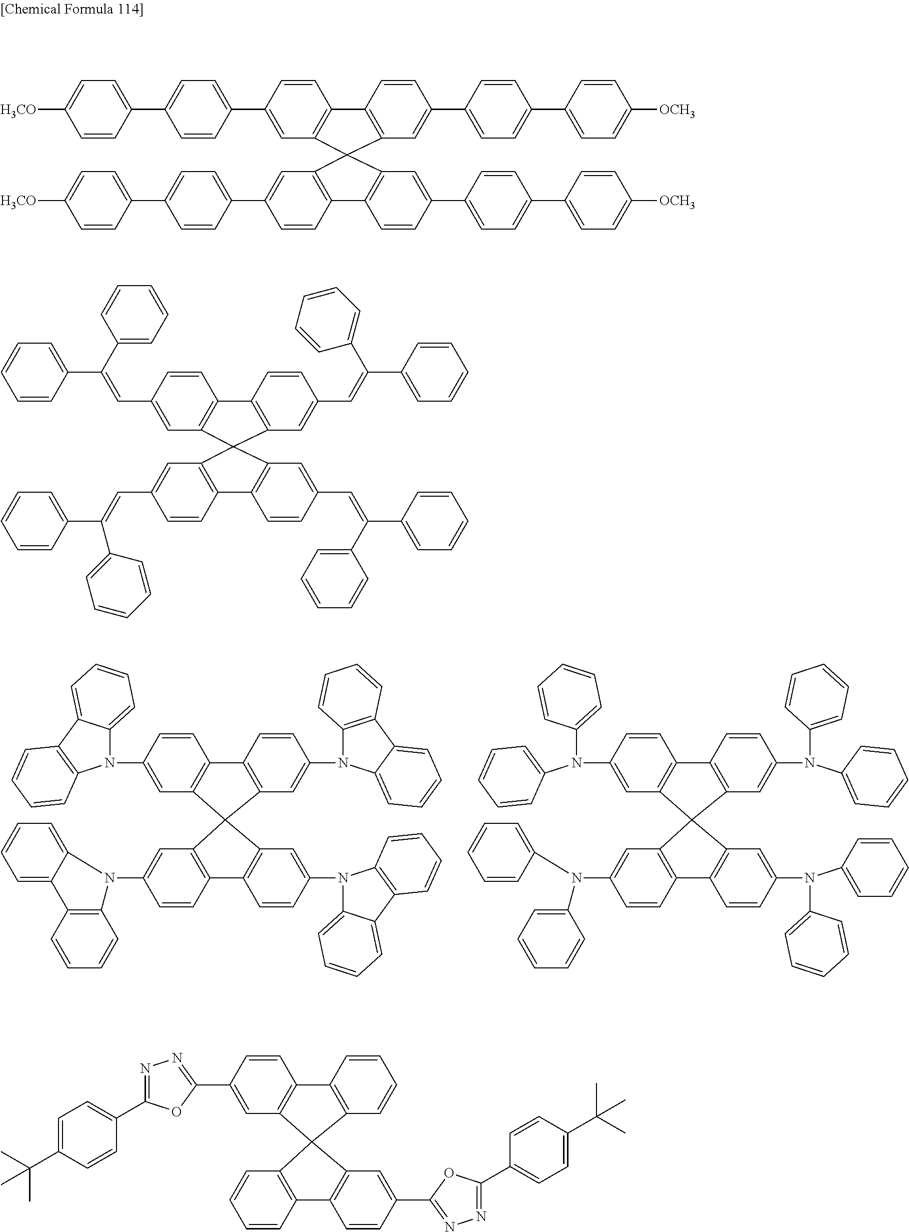

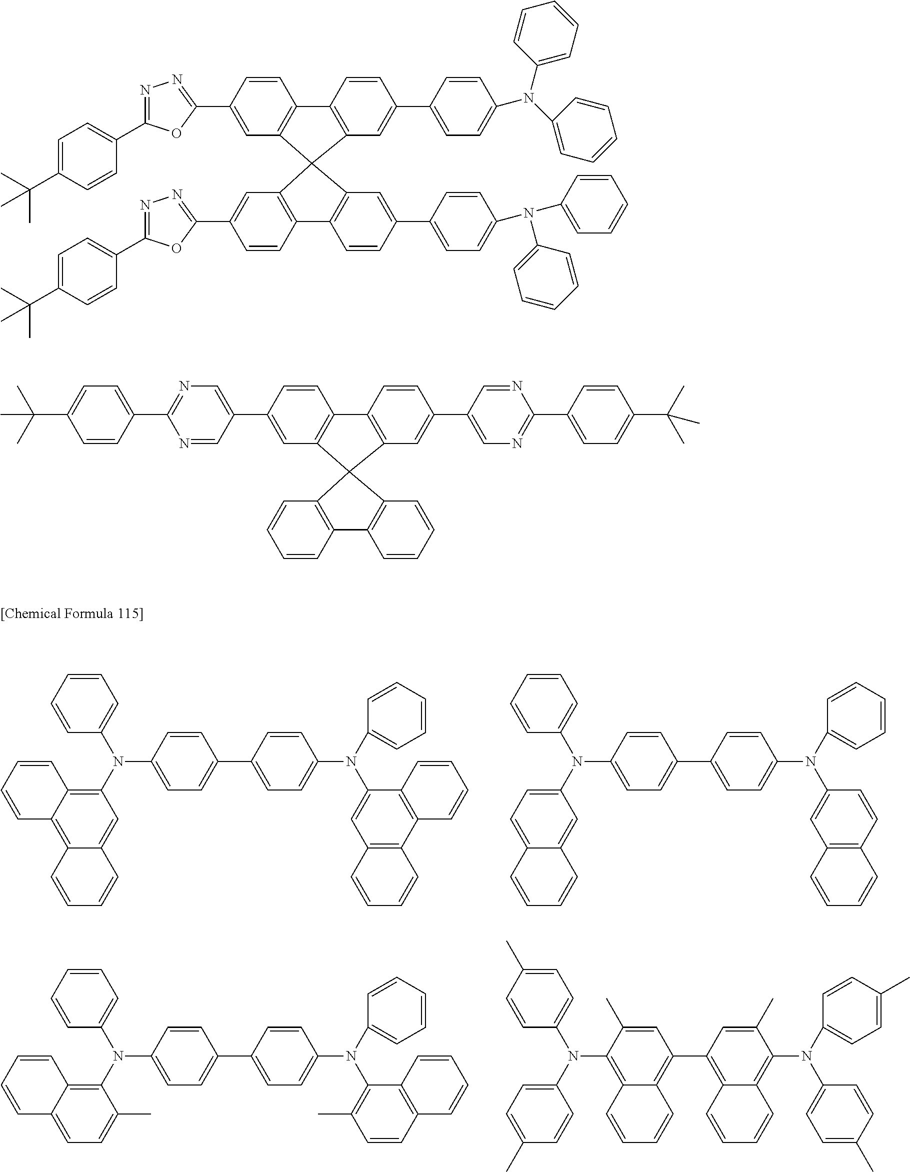

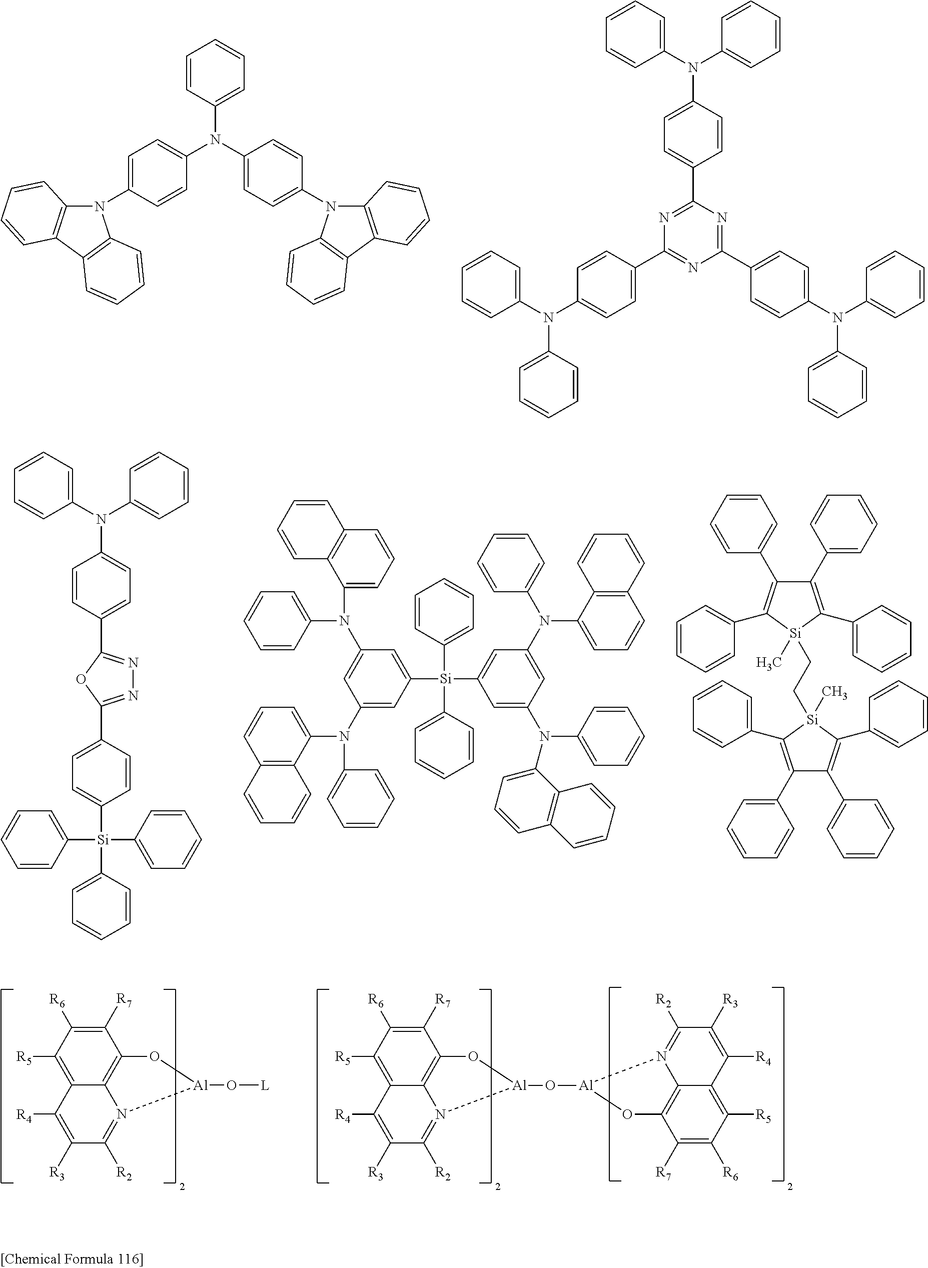

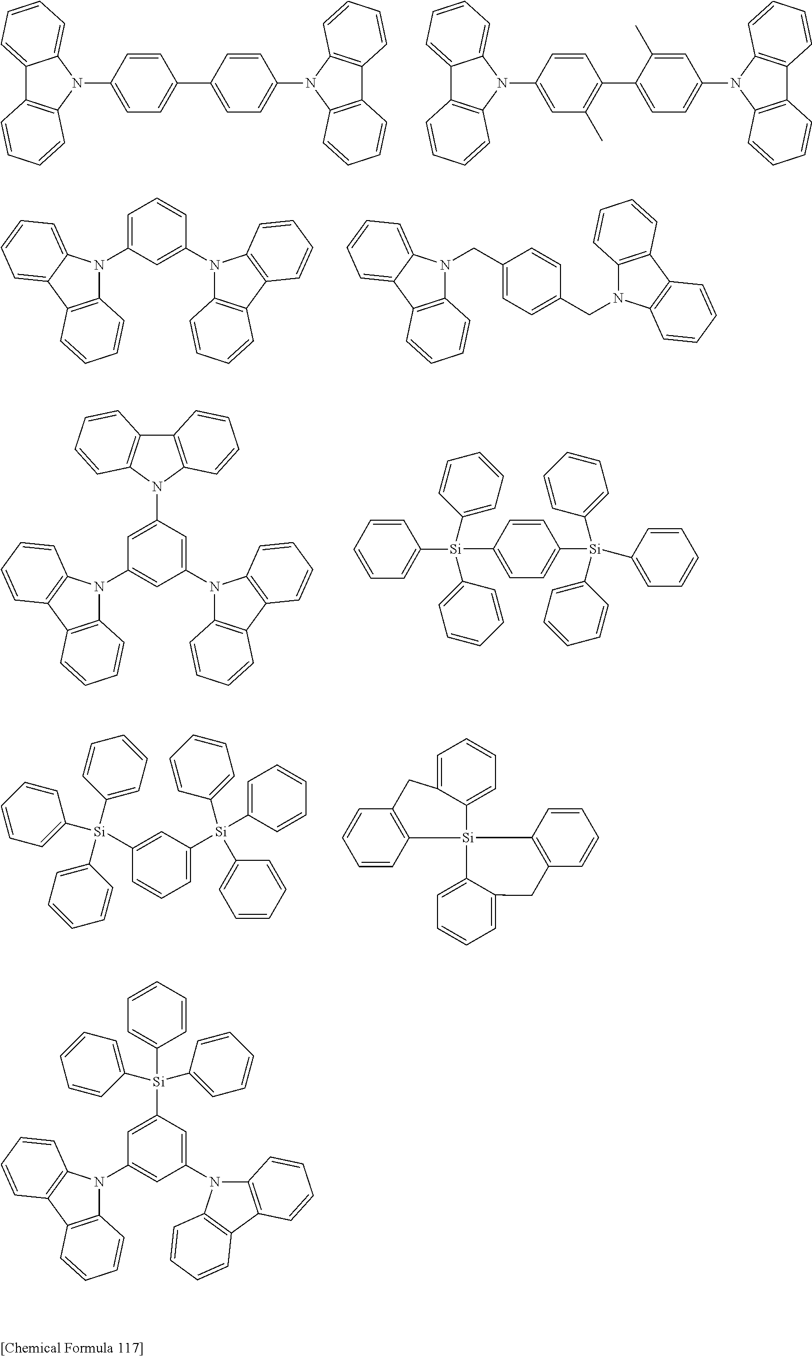

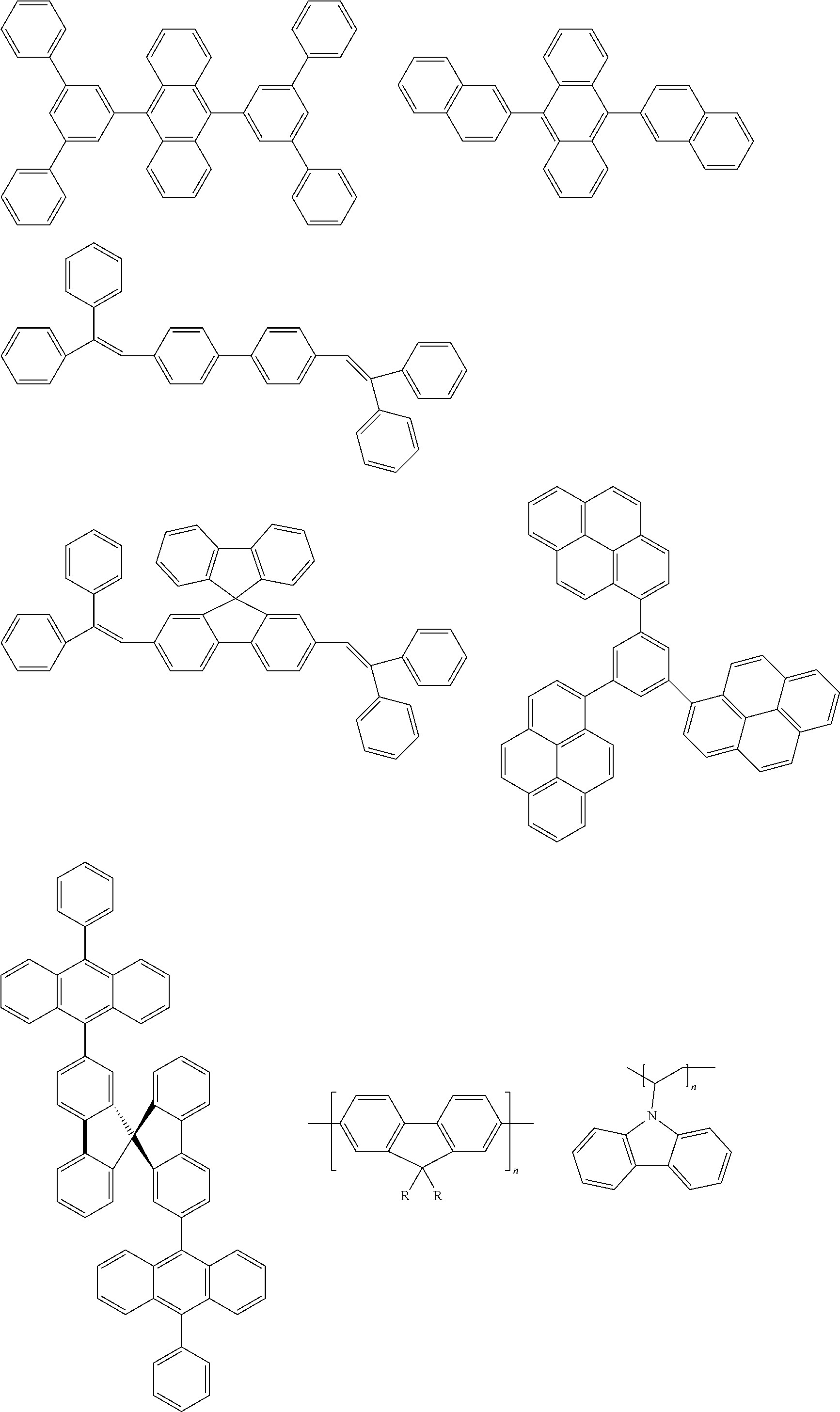

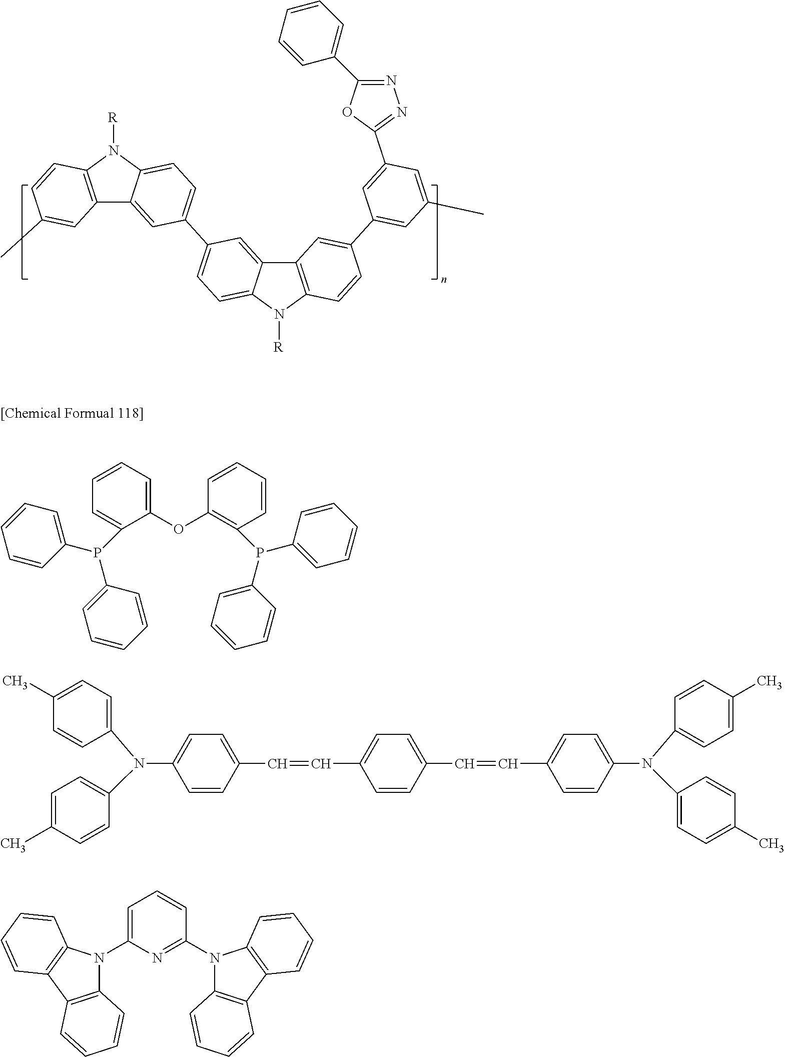

Preferred examples of a compound that may also be used as the material of the hole injection layer are shown below.

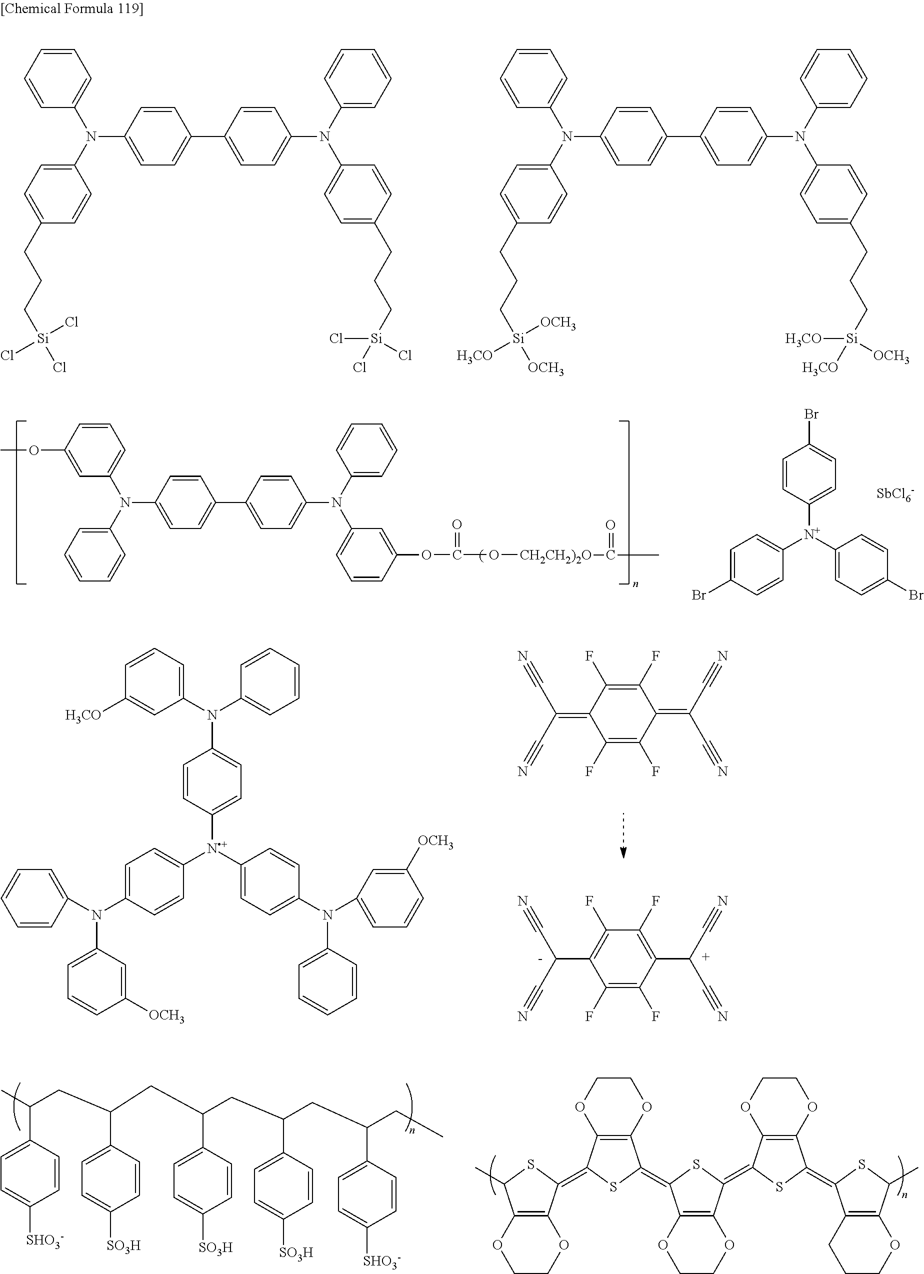

Preferred examples of a compound that may also be used as the material of the hole transport layer are shown below.

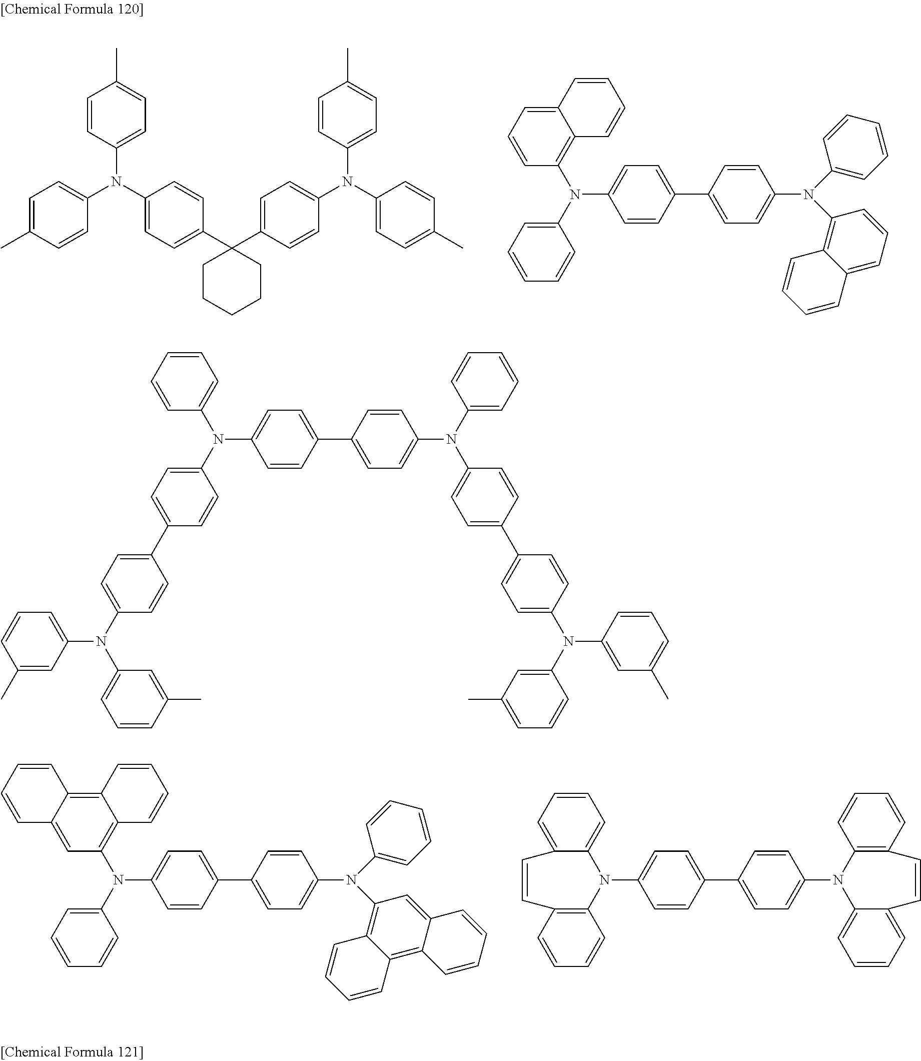

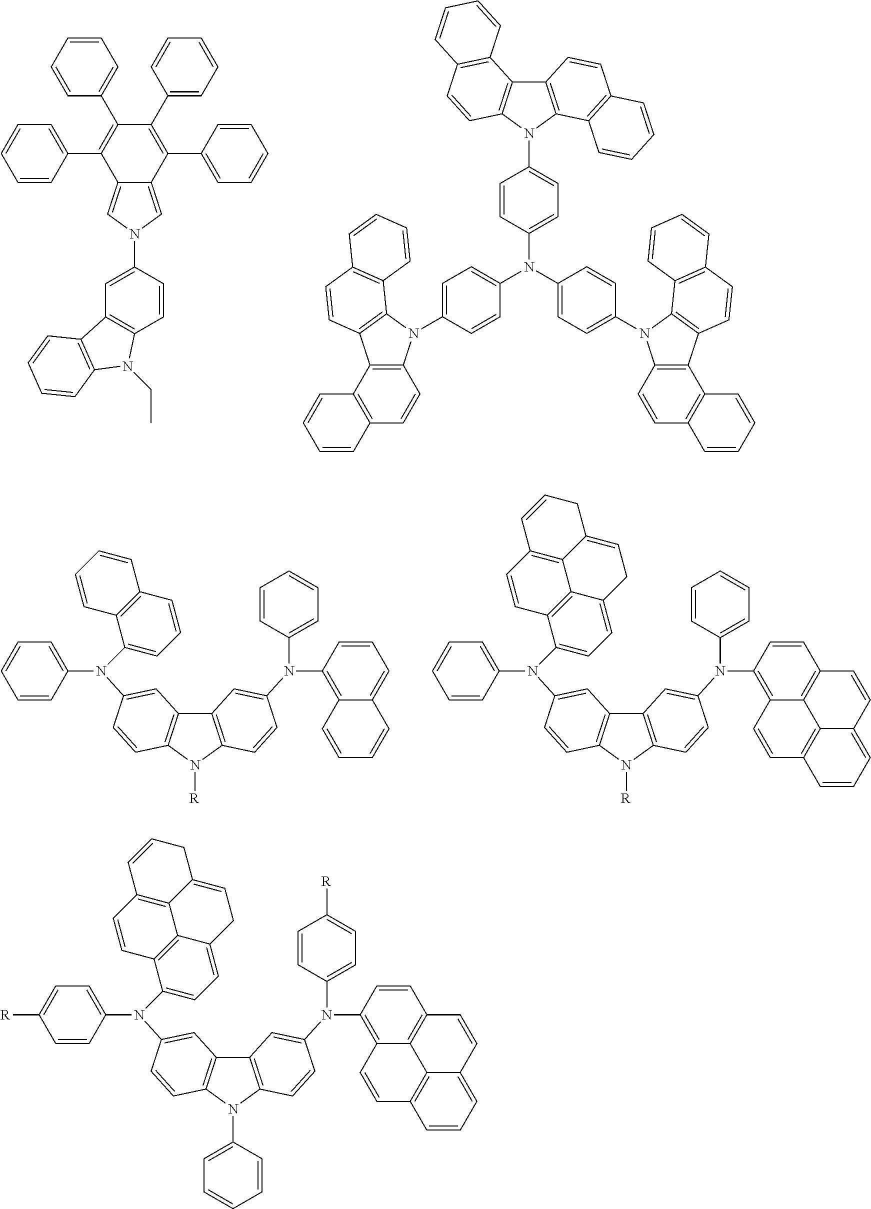

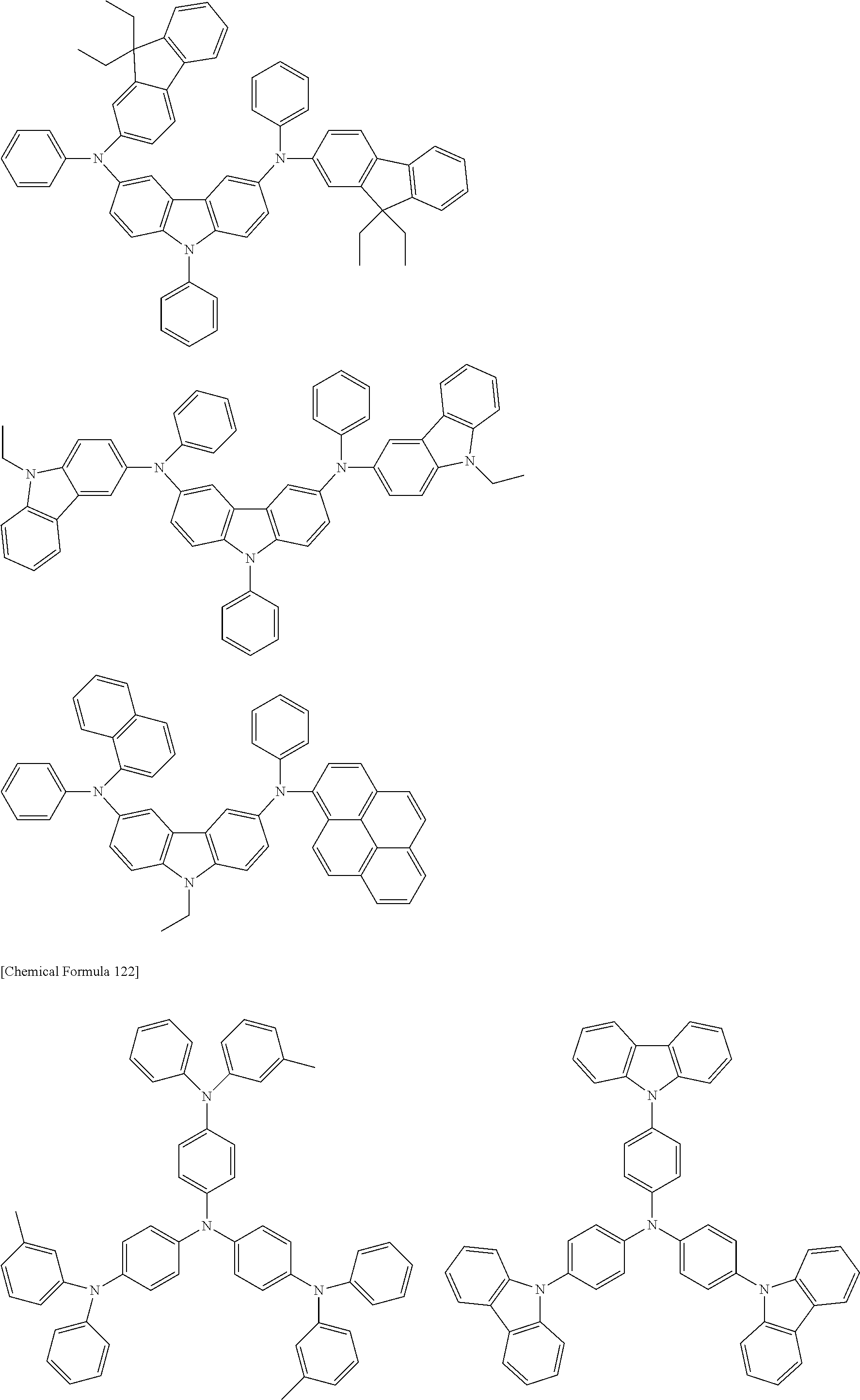

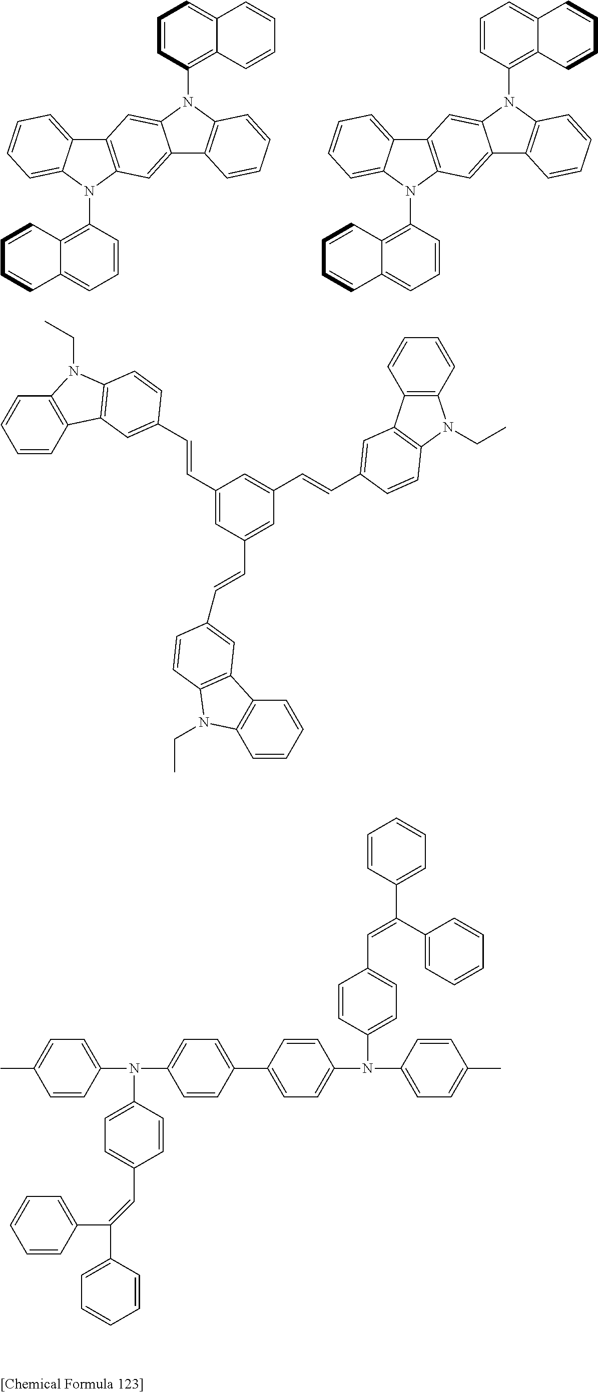

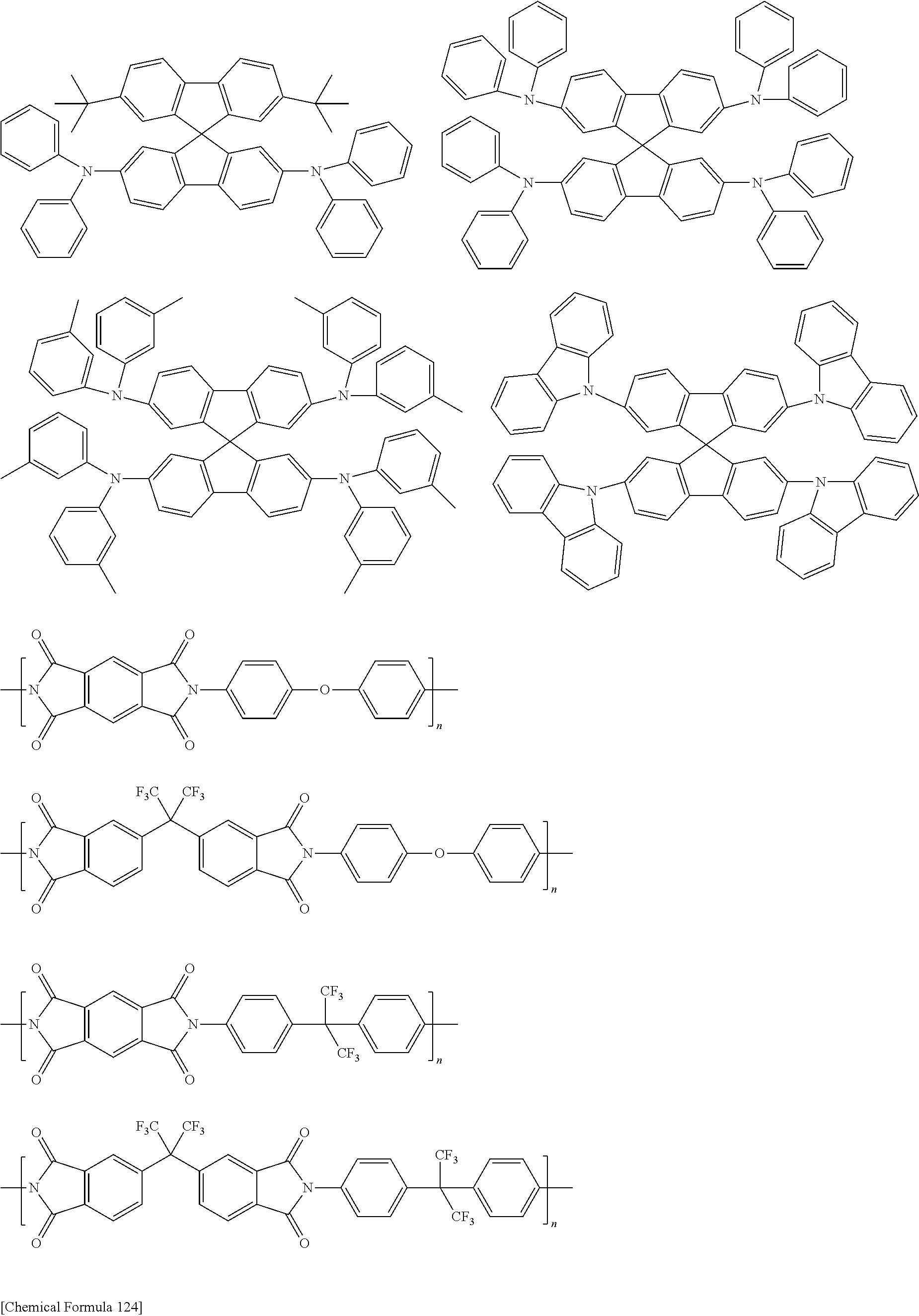

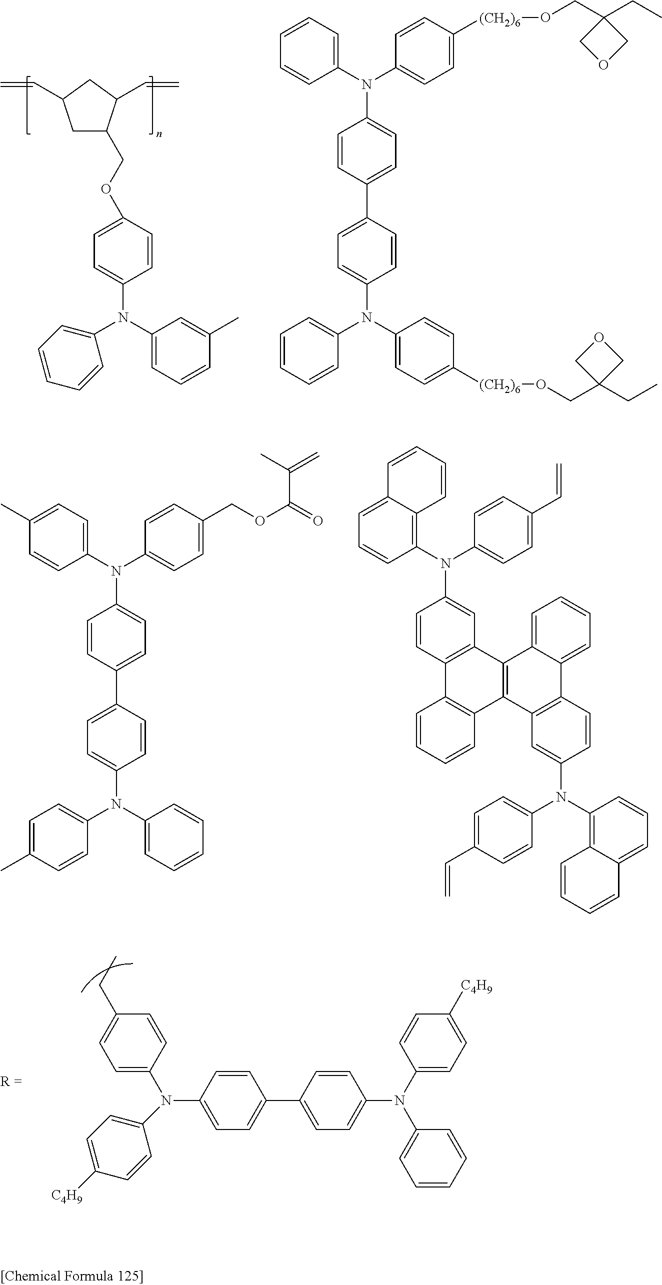

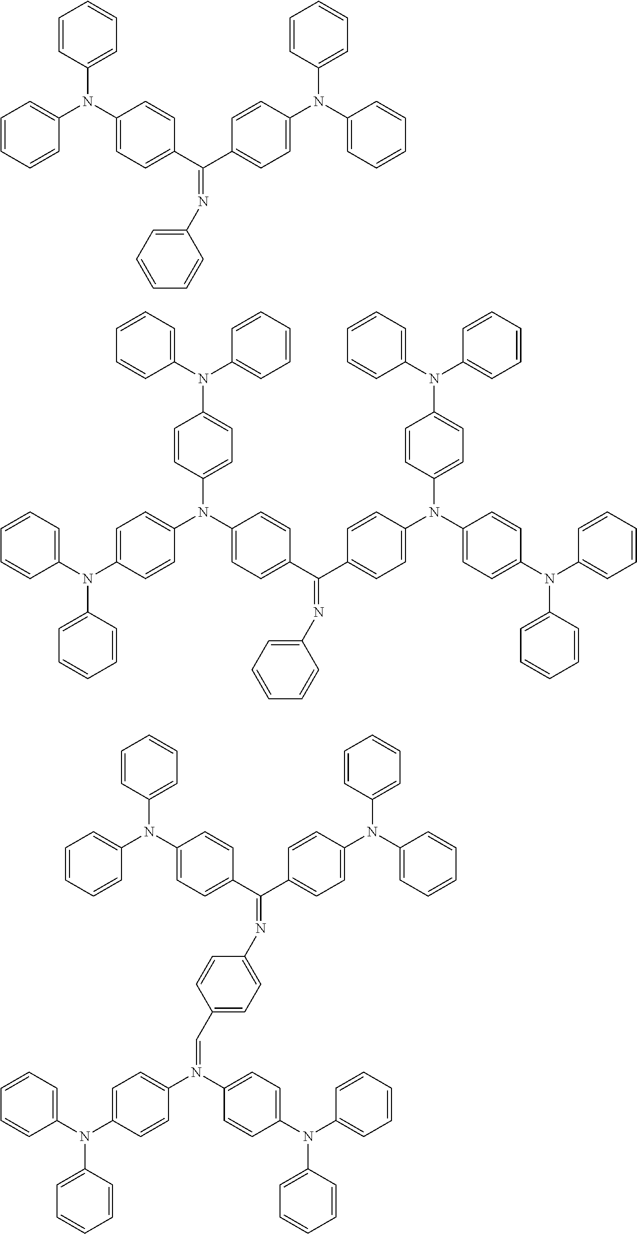

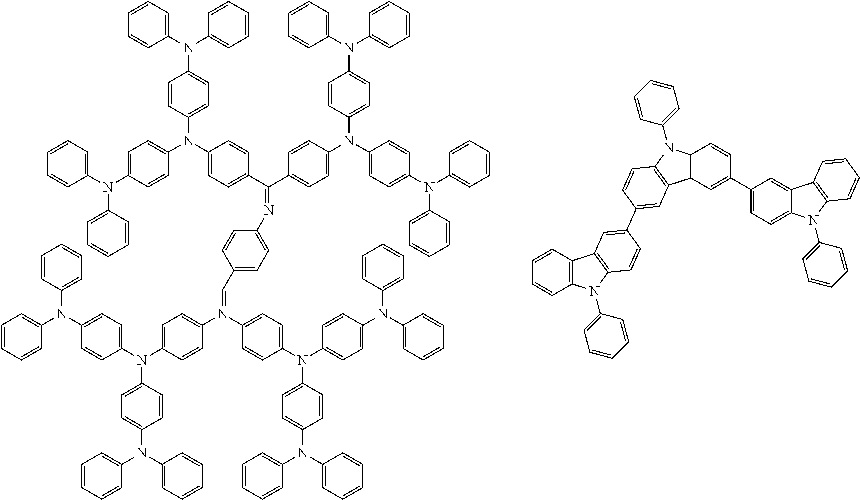

Preferred examples of a compound that may also be used as the material of the electron blocking layer are shown below.

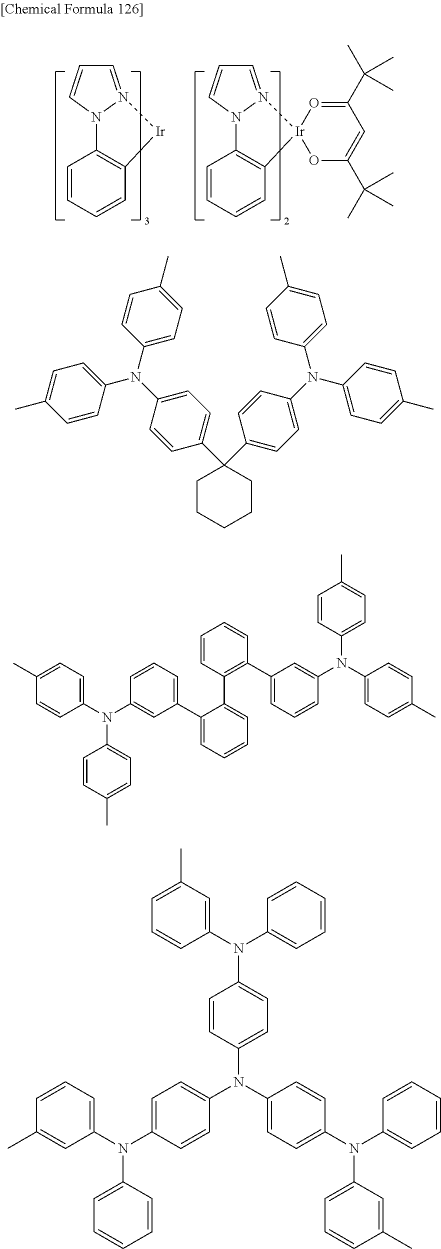

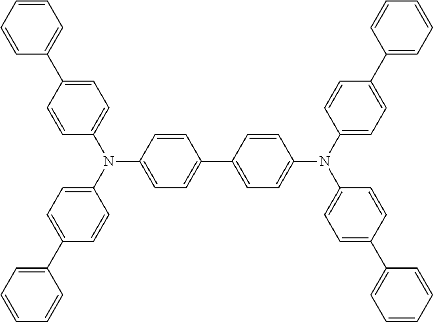 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

Preferred examples of a compound that may also be used as the material of the hole blocking layer are shown below.

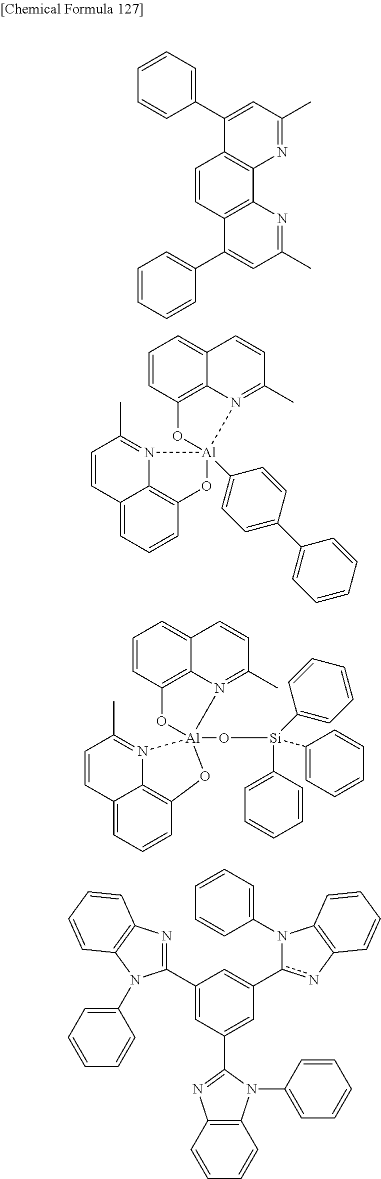

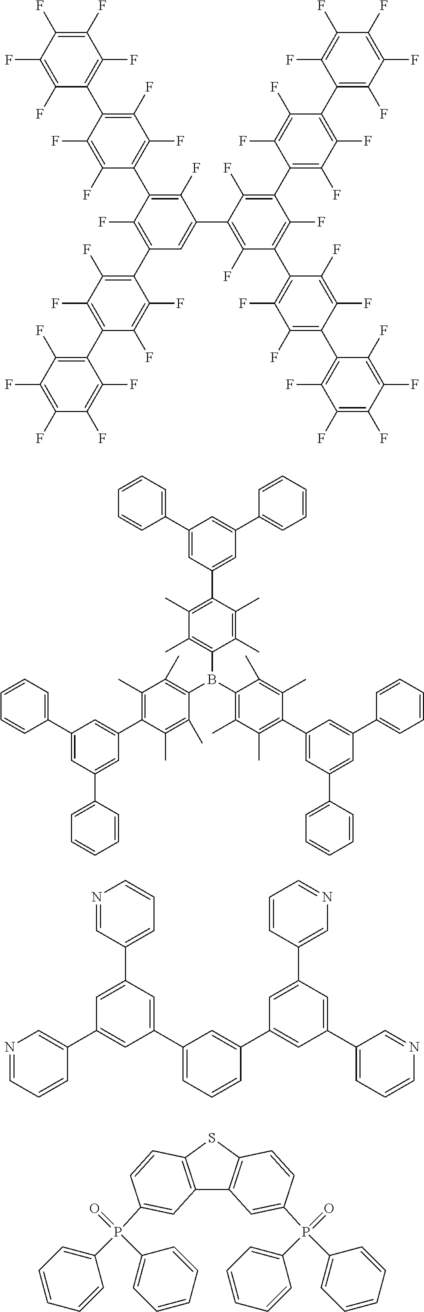

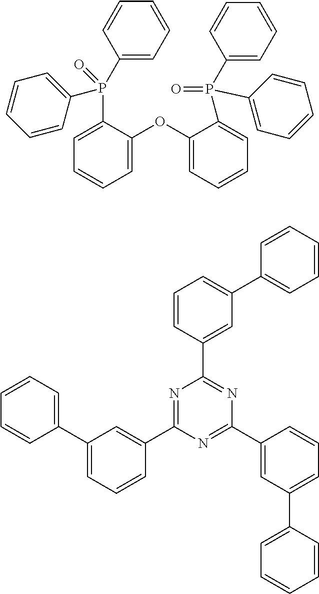 [SMILES: O=P(c1ccccc1)(c1ccccc1)c1ccccc1Oc1ccccc1P(=O)(c1ccccc1)c1ccccc1 ; c1ccc(-c2cccc(-c3nc(-c4cccc(-c5ccccc5)c4)nc(-c4cccc(-c5ccccc5)c4)n3)c2)cc1]

Preferred examples of a compound that may also be used as the material of the electron transport layer are shown below.

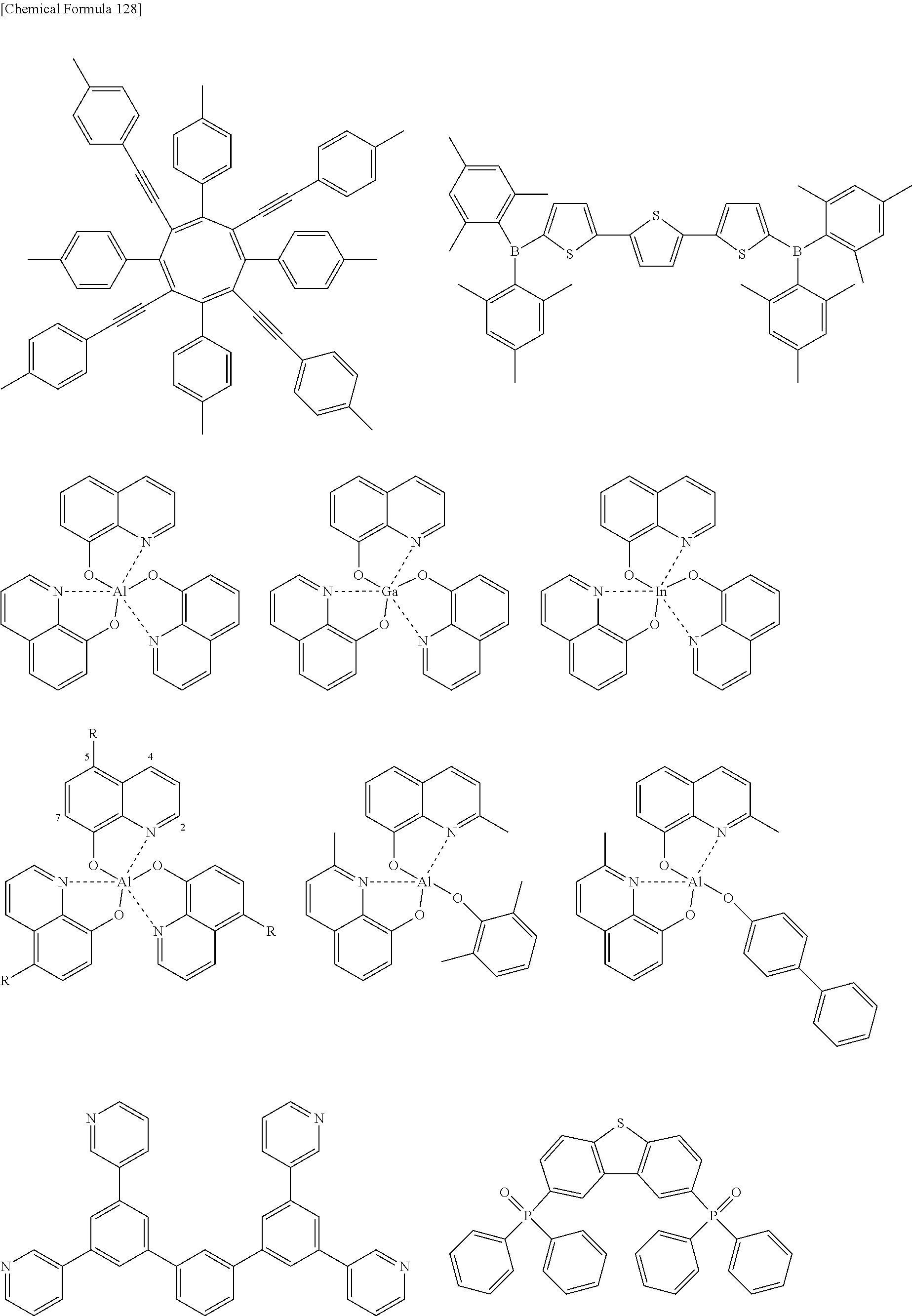

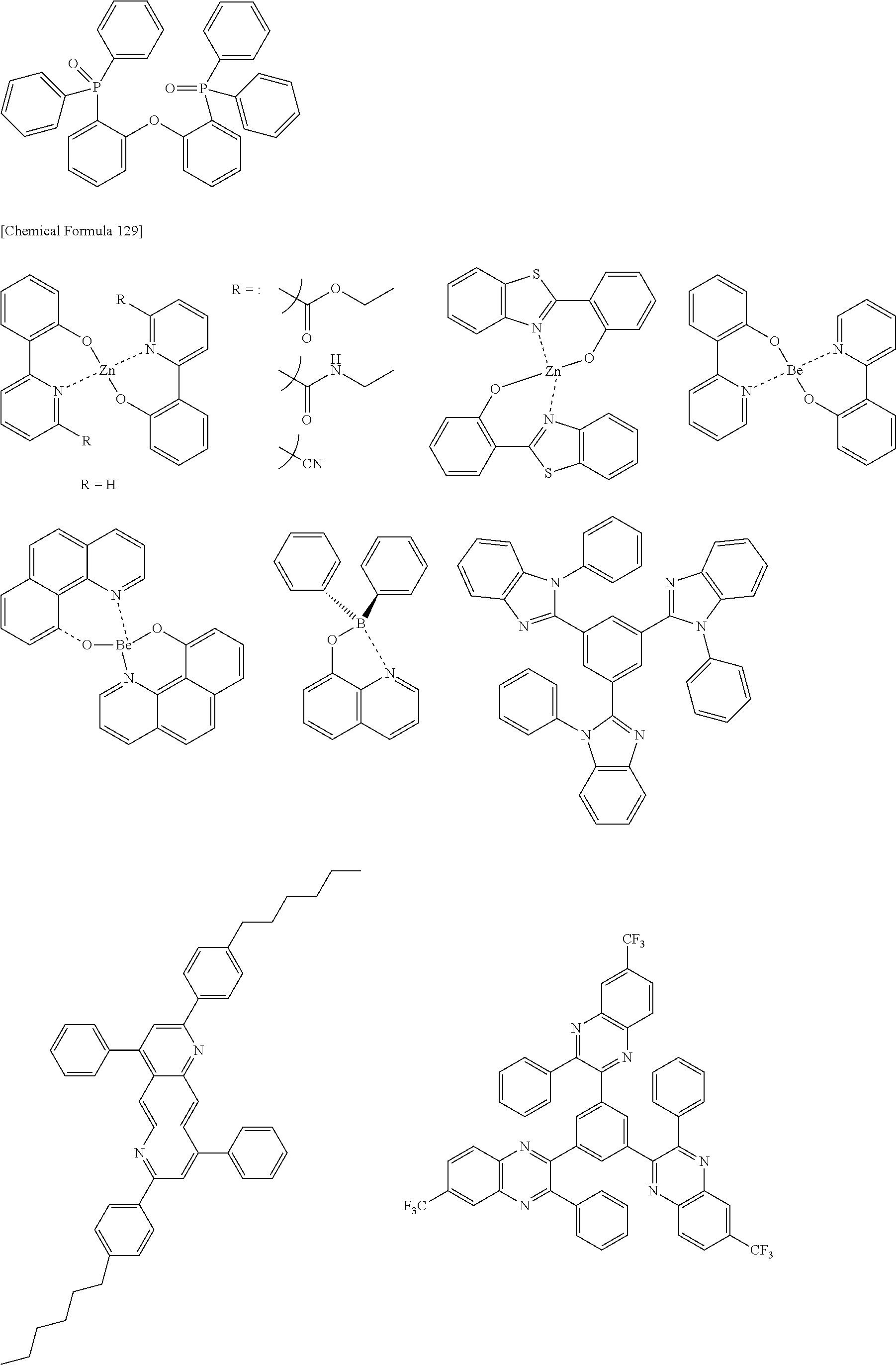

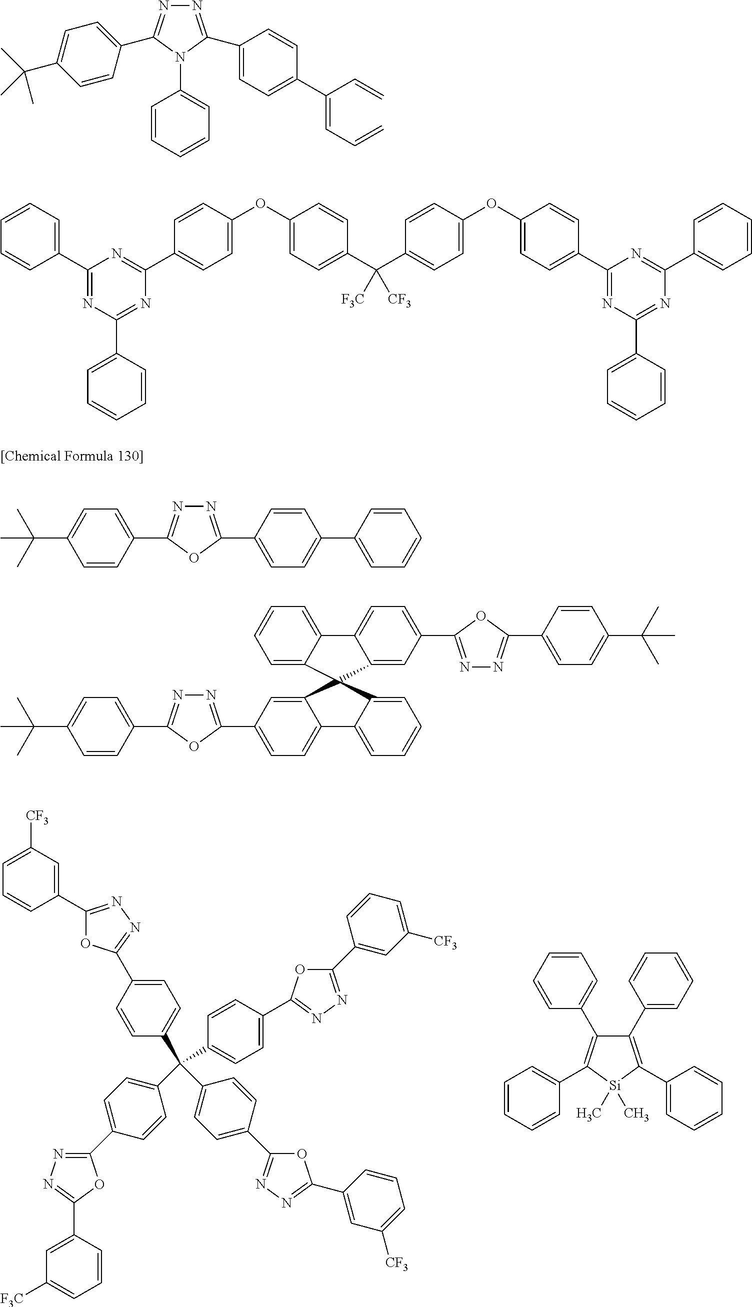

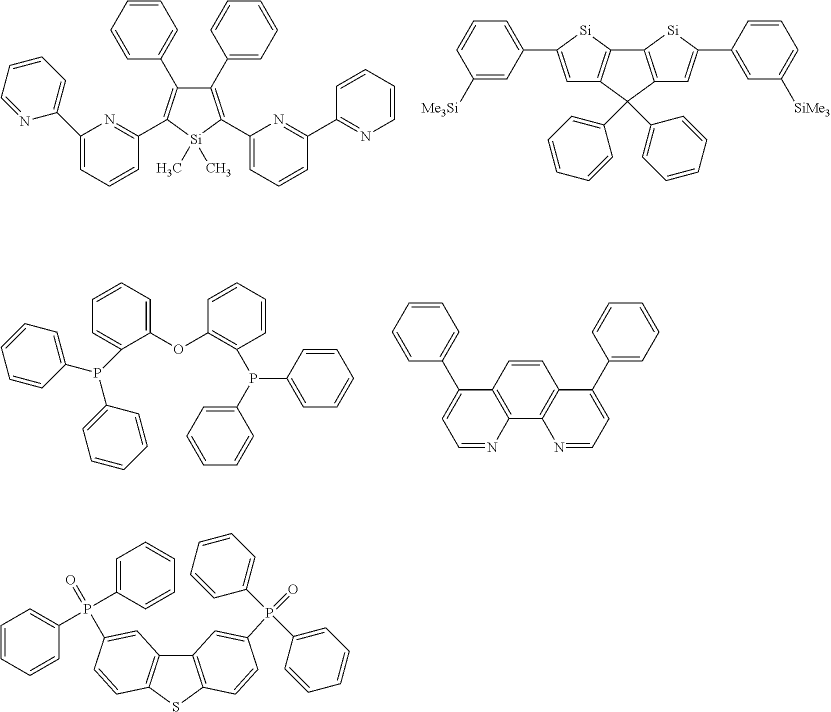

Preferred examples of a compound that may also be used as the material of the electron injection layer are shown below.

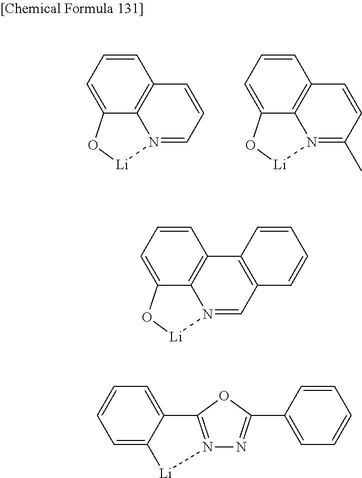

Preferred examples of a compound as a material that may be added are shown below. For example, the compound may be added as a stabilizing material.

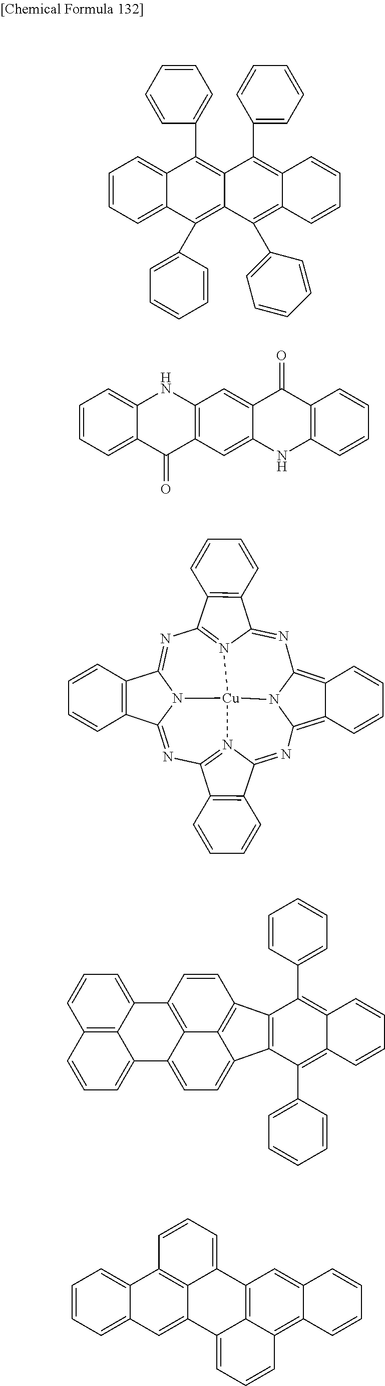

The following describes an embodiment of the present invention in more detail based on Examples. The present invention, however, is not restricted to the following Examples.

### Example 1

### Synthesis of 2,7-bis(phenoxazin-10-yl)triphenylene (Compound 1)

1,2-diiodobenzene (42 g), 3-(trimethylsilyl)phenylboronic acid (52 g), sodium hydroxide (15 g), diglyme (270 mL), and water (70 mL) were added into a nitrogen-substituted reaction vessel and stirred. After adding tetrakis(triphenylphosphine)palladium(0) (7 g), the mixture was heated and refluxed for 15 hours while being stirred. After the mixture was left to cool, water (150 mL) and toluene (80 mL) were added, and an organic layer was collected by liquid separation. After the organic layer was concentrated, purification by silica gel column chromatography was performed to obtain a white powder of 3,3″-bis(trimethylsilyl)-1,1′:2′,1″-terphenyl (yield 70%).

The obtained 3,3″-bis(trimethylsilyl)-1,1′:2′,1″-terphenyl (20 g) and chloroform (80 mL) were added into a nitrogen-substituted reaction vessel, and a chloroform solution (80 mL) of bromine (36 g) was dropped into the vessel while taking a time of 2 hours. After the mixture was stirred at a room temperature for 30 hours, a saturated sodium sulfite aqueous solution (150 mL) was dropped. An organic layer was collected by an extraction procedure using chloroform and concentrated to obtain a crude product. Recrystallization with ethanol was carried out to obtain a white powder of 3,3″-dibromo-1,1′:2′,1″-terphenyl (yield 78%).

The obtained 3,3″-dibromo-1,1′:2′,1″-terphenyl (15 g), molybdenum chloride(V) (30 g), and dichloromethane (100 mL) were added into a nitrogen-substituted reaction vessel and stirred at a room temperature for 20 hours. Water was added, and a precipitate was collected by filtration. The precipitate was dissolved in chloroform (500 mL), and a crude product obtained by concentration after performing purification by adsorption with a silica gel was subjected to washing and purification using methanol to obtain 2,7-dibromotriphenylene (yield 30%).

The obtained 2,7-dibromotriphenylene (1.0 g), phenoxazine (1.5 g), sodium tert-butoxide (0.6 g), tri-tert-butylphosphine (0.1 g), and toluene (80 mL) were added into a nitrogen-substituted reaction vessel. Then, tris(dibenzylideneacetone)palladium-chloroform adduct (0.07 g) was added, and the mixture was heated and refluxed for 10 hours while being stirred. After the mixture was left to cool, methanol was added, and a precipitated crude product was collected by filtration. The crude product was subjected to purification by silica gel column chromatography to obtain a yellow powder of 2,7-bis(phenoxazin-10-yl)triphenylene (Compound 1; yield 80%).

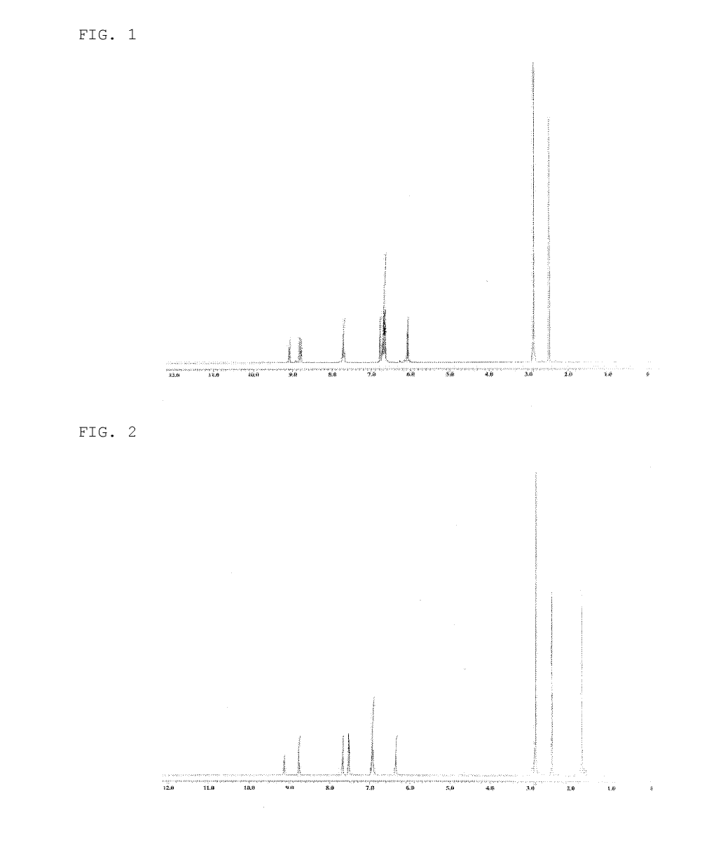

The structure of the obtained yellow powder was identified by NMR. The ¹H-NMR measurement result is shown in FIG. 1.

¹H-NMR (DMSO-d₆) detected 26 hydrogen signals, as follows. δ (ppm)=6.07 (4H), 6.62-6.83 (12H), 7.64-7.76 (4H), 8.79 (2H), 8.83 (2H), 9.08 (2H).

### Example 2

### Synthesis of 2,7-bis(9,9-dimethylacridan-10-yl)triphenylene (Compound 5)

2,7-dibromotriphenylene synthesized in Example 1 (1.0 g), 9,9-dimethylacridan (1.6 g), sodium tert-butoxide (0.6 g), tri-tert-butylphosphine (0.1 g), and toluene (60 mL) were added into a nitrogen-substituted reaction vessel. Then, tris(dibenzylideneacetone)palladium-chloroform adduct (0.07 g) was added, and the mixture was heated and refluxed for 5 hours while being stirred. After the mixture was left to cool, methanol was added, and a precipitated crude product was collected by filtration. The crude product was subjected to purification by silica gel column chromatography to obtain a yellowish white powder of 2,7-bis(9,9-dimethylacridan-10-yl)triphenylene (Compound 5; yield 40%).

The structure of the obtained yellowish white powder was identified by NMR. The ¹H-NMR measurement result is shown in FIG. 2.

¹H-NMR (DMSO-d₆) detected 38 hydrogen signals, as follows. δ (ppm)=1.73 (12H), 6.35 (4H), 6.86-7.04 (8H), 7.53 (4H), 7.62-7.74 (4H), 8.70-8.84 (4H), 9.13 (2H).

### Example 3

### Synthesis of 2-(phenoxazin-10-yl)triphenylene (Compound 20)

2-Bromotriphenylene (2.1 g), phenoxazine (1.8 g), sodium tert-butoxide (0.8 g), tri-tert-butylphosphine (0.2 g), and toluene (80 mL) were added into a nitrogen-substituted reaction vessel. Then, tris(dibenzylideneacetone)palladium-chloroform adduct (0.17 g) was added, and the mixture was heated and refluxed for 3 hours while being stirred. After the mixture was left to cool to a room temperature, the mixture was concentrated under reduced pressure and washed with methanol, followed by purification by silica gel column chromatography to obtain a white powder of 2-(phenoxazin-10-yl)triphenylene (Compound 20; yield 50%).

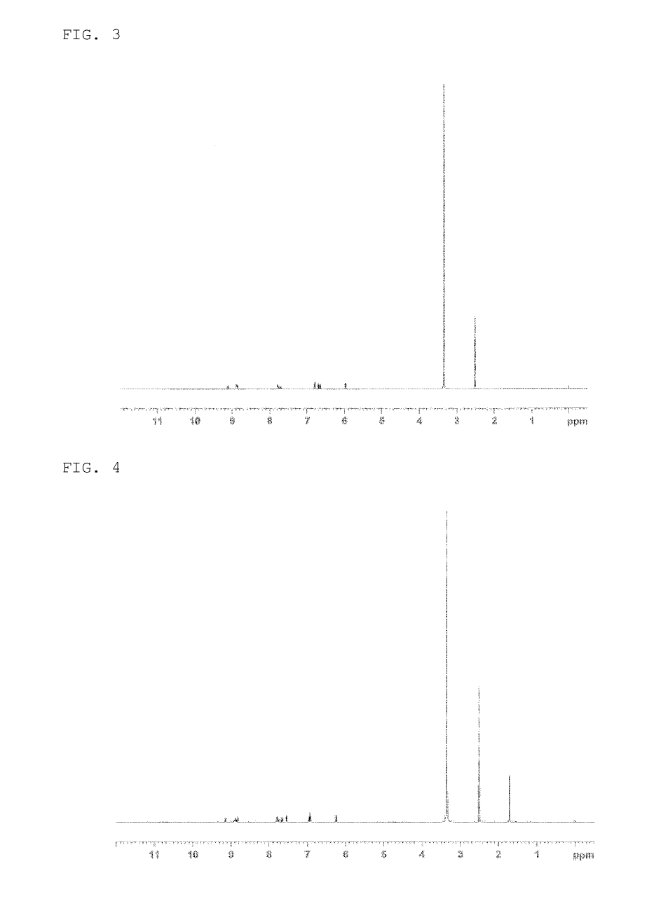

The structure of the obtained white powder was identified by NMR. The ¹H-NMR measurement result is shown in FIG. 3.

¹H-NMR (DMSO-d₆) detected 19 hydrogen signals, as follows. δ (ppm)=5.97 (2H), 6.64 (2H), 6.70 (2H), 6.79 (2H), 7.75-7.85 (5H), 8.79-9.00 (5H), 9.11 (1H).

### Example 4

### Synthesis of 2-(9,9-dimethylacridan-10-yl)triphenylene (Compound 21)

2-Bromotriphenylene (2.1 g), 9,9-dimethylacridane (1.8 g), sodium tert-butoxide (0.8 g), tri-tert-butylphosphine (0.2 g), and toluene (80 mL) were added into a nitrogen-substituted reaction vessel. Then, tris(dibenzylideneacetone)palladium-chloroform adduct (0.17 g) was added, and the mixture was heated and refluxed for 4 hours while being stirred. After the mixture was left to cool to a room temperature, the mixture was concentrated under reduced pressure and washed with methanol, followed by purification by silica gel column chromatography to obtain a white powder of 2-(9,9-dimethylacridan-10-yl)triphenylene (Compound 21; yield 55%).

The structure of the obtained white powder was identified by NMR. The ¹H-NMR measurement result is shown in FIG. 4.

¹H-NMR (DMSO-d₆) detected 25 hydrogen signals, as follows. δ (ppm)=1.72 (6H), 6.24 (2H), 6.87-7.05 (4H), 7.55 (2H), 7.65 (1H), 7.68 (1H), 7.69-7.90 (3H), 8.75-8.99 (5H), 9.14 (1H).

### Example 5

### Synthesis of 2-{3-(diphenylamino)carbazol-10-yl}triphenylene (Compound 24)

2-Bromotriphenylene (2.1 g), 3-(diphenylamino)carbazole (3.7 g), sodium tert-butoxide (0.8 g), tri-tert-butylphosphine (0.4 g), and xylene (80 mL) were added into a nitrogen-substituted reaction vessel. Then, tris(dibenzylideneacetone)palladium-chloroform adduct (0.34 g) was added, and the mixture was heated and refluxed for 8 hours while being stirred. After the mixture was left to cool to a room temperature, the mixture was concentrated under reduced pressure and washed with methanol, followed by purification by silica gel column chromatography to obtain a white powder of 2-{3-(diphenylamino)carbazol-10-yl}triphenylene (Compound 24; yield 33%).

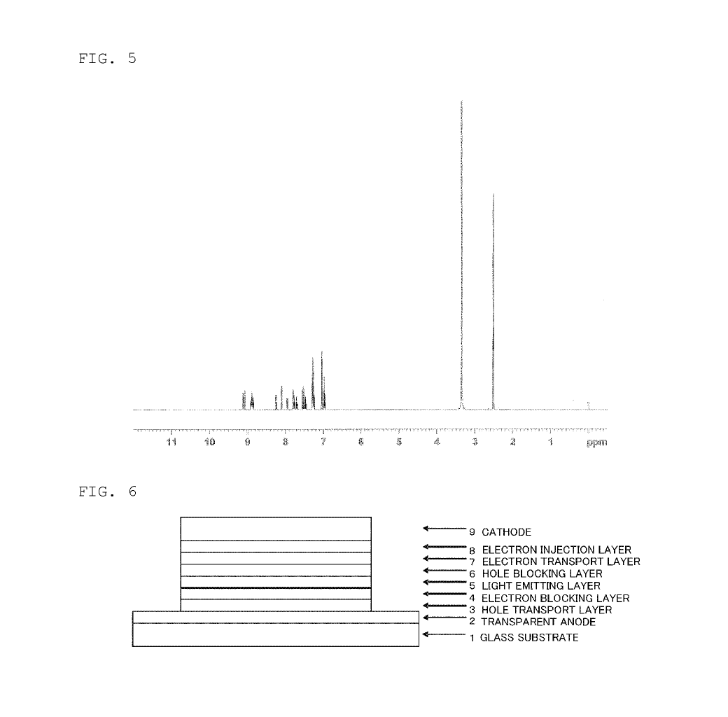

The structure of the obtained white powder was identified by NMR. The ¹H-NMR measurement result is shown in FIG. 5.

¹H-NMR (DMSO-d₆) detected 28 hydrogen signals, as follows. δ (ppm)=6.96 (2H), 7.03 (2H), 7.04 (2H), 7.22-7.32 (6H), 7.43-7.57 (3H), 7.70 (1H), 7.72-7.83 (3H), 7.95 (1H), 8.10 (1H), 8.24 (1H), 8.82-8.94 (4H), 9.06 (1H), 9.11 (1H).

### Example 6

A 100 nm-thick vapor-deposited film was fabricated on an ITO substrate using the compounds of the present invention. The work function was measured using an atmosphere photoelectron spectrometer (AC-3 produced by Riken Keiki Co., Ltd.).

| | Work function |
|---|---|
| Compound of Example 1 of the present invention | 5.70 eV |
| Compound of Example 2 of the present invention | 5.90 eV |
| CBP | 6.00 eV |

Thus, the compounds of the present invention have a preferable energy level as a light emitting layer material, which is about the same as that of CBP used as a common light emission host.

Further, the compounds of the present invention have work functions greater than the work function 5.4 eV of common hole transport materials such as NPD and TPD, and have high hole blocking capability.

### Example 7

A 10⁻⁵ mol/L toluene solution was prepared for the compound of Example 1 of the present invention (Compound 1). This toluene solution was irradiated with ultraviolet light at 300 K while being aerated with nitrogen, and fluorescence having a peak wavelength of 469 nm was observed.

The time-resolved spectrum of the above toluene solution was also measured before and after the aeration of nitrogen by using a compact fluorescence lifetime spectrometer (Quantaurus-tau produced by Hamamatsu Photonics K.K.). As a result, fluorescence having a light emission lifetime of 0.0029 μs and delayed fluorescence having light emission lifetimes of 0.0082 μs and 0.716 μs were observed.

The photoluminescence (hereinafter referred to as PL) quantum yield of the above toluene solution was also measured before and after the aeration of nitrogen by using an absolute PL quantum yields measurement system (Quantaurus-QY produced by Hamamatsu Photonics K.K.) at 300 K. As a result, the PL quantum yield before the aeration of nitrogen was 7.1%, and the PL quantum yield after the aeration of nitrogen was 11.6%.

### Example 8

A 10⁻⁵ mol/L toluene solution was prepared for the compound of Example 2 of the present invention (Compound 5) instead of the compound of Example 1 of the present invention (Compound 1) in Example 7, and the characteristics of the toluene solution were measured in the same manner as Example 7. As a result, fluorescence having a peak wavelength of 419 nm was observed, and fluorescence having a light emission lifetime of 0.00482 μs and delayed fluorescence having light emission lifetimes of 0.0154 μs and 0.187 μs were observed.

The PL quantum yield before the aeration of nitrogen was 11.3%, and the PL quantum yield after the aeration of nitrogen was 15.8%.

### Example 9

A 10⁻⁵ mol/L toluene solution was prepared for the compound of Example 3 of the present invention (Compound 20) instead of the compound of Example 1 of the present invention (Compound 1) in Example 7, and the characteristics of the toluene solution were measured in the same manner as Example 7. As a result, fluorescence having a peak wavelength of 441 nm was observed, and fluorescence having a light emission lifetime of 0.0035 μs and delayed fluorescence having a light emission lifetime of 0.0091 μs were observed.

The PL quantum yield before the aeration of nitrogen was 4.5%, and the PL quantum yield after the aeration of nitrogen was 6.2%.

### Example 10

A 10⁻⁵ mol/L toluene solution was prepared for the compound of Example 4 of the present invention (Compound 21) instead of the compound of Example 1 of the present invention (Compound 1) in Example 7, and the characteristics of the toluene solution were measured in the same manner as Example 7. As a result, fluorescence having a peak wavelength of 390 nm was observed, and fluorescence having a light emission lifetime of 0.0085 μs and delayed fluorescence having a light emission lifetime of 0.0265 μs were observed.

The PL quantum yield before the aeration of nitrogen was 14.7%, and the PL quantum yield after the aeration of nitrogen was 23.7%.

### Example 11

A 10⁻⁵ mol/L toluene solution was prepared for the compound of Example 5 of the present invention (Compound 24) instead of the compound of Example 1 of the present invention (Compound 1) in Example 7, and the characteristics of the toluene solution were measured in the same manner as Example 7. As a result, fluorescence having a peak wavelength of 412 nm was observed, and fluorescence having a light emission lifetime of 0.0046 μs and delayed fluorescence having a light emission lifetime of 0.15 μs were observed.

The PL quantum yield before the aeration of nitrogen was 11.9%, and the PL quantum yield after the aeration of nitrogen was 16.1%.

### Example 12

On a glass substrate, dual vapor deposition of mCBP of the following structural formula and the compound of Example 1 of the present invention (Compound 1) was performed at a vapor deposition rate ratio of 94:6 (mCBP:compound of Example 1 of the present invention (Compound 1)). Thus, an organic PL device was obtained. The device was irradiated with light at 337 nm with N₂ laser through the use of an absolute PL quantum yields measurement system (Quantaurus-QY produced by Hamamatsu Photonics K.K.). An emission spectrum from the thin film upon the irradiation was measured at 300 K under a nitrogen stream. As a result, the PL quantum yield was 32.7%.

Next, the device was irradiated with light at 337 nm with N₂ laser, and a time-resolved spectrum upon the irradiation was evaluated with a streak camera (C4334 produced by Hamamatsu Photonics K.K.). A component having a light emission lifetime of 115 μs or less and a component having a light emission lifetime of more than 115 μs were determined as fluorescence and delayed fluorescence, respectively. As a result, the light emission of the device included 40% of a fluorescence component and 60% of a delayed fluorescence component.

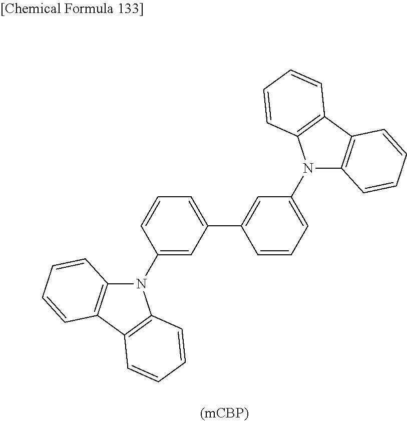 [SMILES: c1cc(-c2cccc(-n3c4ccccc4c4ccccc43)c2)cc(-n2c3ccccc3c3ccccc32)c1]

### Example 13

The organic EL device, as shown in FIG. 6, was fabricated by vapor-depositing a hole transport layer 3, an electron blocking layer 4, a light emitting layer 5, a hole blocking layer 6, an electron transport layer 7, an electron injection layer 8, and a cathode (aluminum electrode) 9 in this order on a glass substrate 1 on which an ITO electrode was formed as a transparent anode 2 beforehand.

Specifically, the glass substrate 1 having ITO (a film thickness of 100 nm) formed thereon was washed with an organic solvent, and subjected to a UV ozone treatment to wash the surface. The glass substrate with the ITO electrode was then installed in a vacuum vapor deposition apparatus, and the pressure was reduced to 0.001 Pa or less.

This was followed by formation of the hole transport layer 3 by vapor depositing NPD over the transparent anode 2 in a film thickness of 30 nm at a vapor deposition rate of 2.0 Å/sec. The electron blocking layer 4 was then formed on the hole transport layer 3 by forming mCP in a film thickness of 10 nm at 2.0 Å/sec. Then, the light emitting layer 5 was formed on the electron blocking layer 4 in a film thickness of 15 nm by dual vapor deposition of DPEPO of the following structural formula and the compound of Example 1 of the present invention (Compound 1) at a vapor deposition rate ratio of 94:6 (DPEPO:compound of Example 1 of the present invention (Compound 1)). The hole blocking layer 6 was then formed on the light emitting layer 5 by forming DPEPO of the following structural formula in a film thickness of 10 nm at 2.0 Å/sec. The electron transport layer 7 was then formed on the hole blocking layer 6 by forming TPBI in a film thickness of 40 nm at a vapor deposition rate of 2.0 Å/sec. The electron injection layer 8 was then formed on the electron transport layer 7 by forming lithium fluoride in a film thickness of 0.8 nm at a vapor deposition rate of 0.1 Å/sec. Finally, the cathode 9 was formed by vapor depositing aluminum in a film thickness of 100 nm. The characteristics of the organic EL device thus fabricated were measured in the atmosphere at an ordinary temperature.

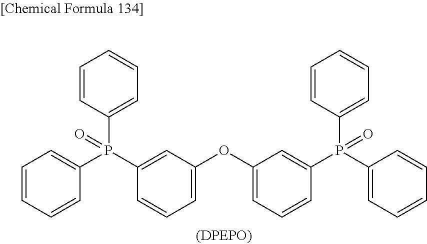 [SMILES: O=P(c1ccccc1)(c1ccccc1)c1cccc(Oc2cccc(P(=O)(c3ccccc3)c3ccccc3)c2)c1]

The maximum external quantum efficiency was 5.9% when applying a DC voltage to the organic EL device fabricated using the compound of Example 1 of the present invention (Compound 1).

As shown in the above results, the compounds having a triphenylene ring structure of the present invention can emit delayed fluorescence and have a high PL quantum yield. Further, as the above result demonstrates, the organic EL devices using the compounds of the present invention have a high external quantum efficiency.

### INDUSTRIAL APPLICABILITY

The compounds having a triphenylene ring structure of the present invention can emit delayed fluorescence and have desirable thin-film stability, and the compounds are therefore excellent as material of a light emitting layer, especially as a dopant material of a light emitting layer. The organic EL devices produced by using the compounds can have greatly improved luminance and luminous efficiency over conventional organic EL devices.

### DESCRIPTION OF REFERENCE NUMERAL

1 Glass substrate
2 Transparent anode
3 Hole transport layer
4 Electron blocking layer
5 Light emitting layer
6 Hole blocking layer
7 Electron transport layer
8 Electron injection layer
9 Cathode

1. A compound of the following general formula (1) having a triphenylene ring structure, 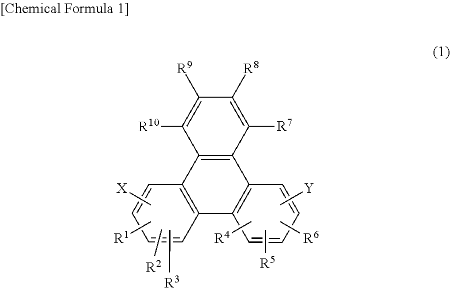
wherein X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group; Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group; and R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

2. The compound having a triphenylene ring structure according to claim 1, wherein the compound is represented by the following general formula (1-1), 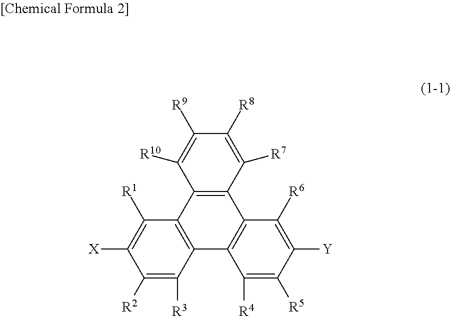
wherein X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group; Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group; and R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

3. The compound having a triphenylene ring structure according to claim 1, wherein the compound is represented by the following general formula (1-2), 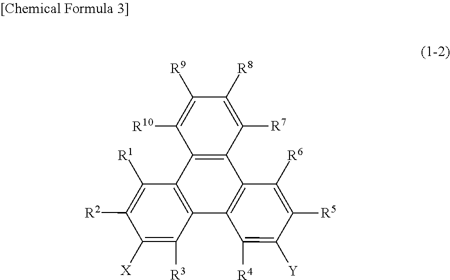
wherein X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group; Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group; and R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

4. The compound having a triphenylene ring structure according to claim 1, wherein the compound is represented by the following general formula (1-3), 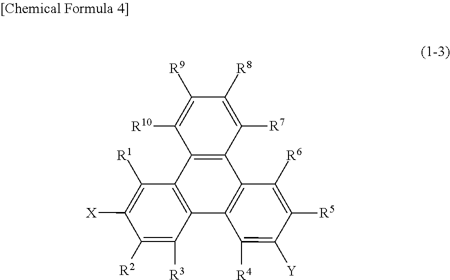
wherein X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group; Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group; and R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

5. The compound having a triphenylene ring structure according to claim 1, wherein the compound is represented by the following general formula (1-4), 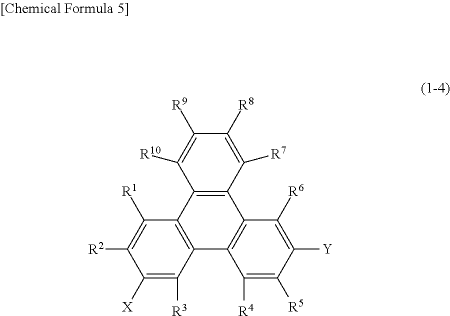
wherein X represents a substituted or unsubstituted aromatic hydrocarbon group, a substituted or unsubstituted aromatic heterocyclic group, a substituted or unsubstituted condensed polycyclic aromatic group, or a disubstituted amino group substituted with a group selected from an aromatic hydrocarbon group, an aromatic heterocyclic group, and a condensed polycyclic aromatic group; Y represents a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group; and R¹ to R¹⁰ may be the same or different, and represent a hydrogen atom, a deuterium atom, a fluorine atom, a chlorine atom, cyano, nitro, linear or branched alkyl of 1 to 6 carbon atoms that may have a substituent, cycloalkyl of 5 to 10 carbon atoms that may have a substituent, linear or branched alkenyl of 2 to 6 carbon atoms that may have a substituent, linear or branched alkyloxy of 1 to 6 carbon atoms that may have a substituent, cycloalkyloxy of 5 to 10 carbon atoms that may have a substituent, a substituted or unsubstituted aromatic hydrocarbon group, a substituted or unsubstituted aromatic heterocyclic group, a substituted or unsubstituted condensed polycyclic aromatic group, substituted or unsubstituted aryloxy, or a disubstituted amino group substituted with a group selected from an aromatic hydrocarbon group, an aromatic heterocyclic group, and a condensed polycyclic aromatic group, where R¹ to R¹⁰ may bind to each other via a single bond, substituted or unsubstituted methylene, an oxygen atom, or a sulfur atom to form a ring.

6. The compound having a triphenylene ring structure according to claim 1, wherein X in the general formula (1), (1-1), (1-2), (1-3), or (1-4) is a monovalent group selected from substituted or unsubstituted phenoxazinyl, substituted or unsubstituted phenothiazinyl, substituted or unsubstituted acridinyl, substituted or unsubstituted phenazinyl, and carbazolyl having, as a substituent, a disubstituted amino group substituted with an aromatic hydrocarbon group or a condensed polycyclic aromatic group.

7. The compound having a triphenylene ring structure according to claim 1, wherein Y in the general formula (1), (1-1), (1-2), (1-3), or (1-4) is a monovalent group selected from substituted or unsubstituted phenoxazinyl, substituted or unsubstituted phenothiazinyl, substituted or unsubstituted acridinyl, substituted or unsubstituted phenazinyl, and carbazolyl having, as a substituent, a disubstituted amino group substituted with an aromatic hydrocarbon group or a condensed polycyclic aromatic group.

8. The compound having a triphenylene ring structure according to claim 1, wherein X and Y in the general formula (1), (1-1), (1-2), (1-3), or (1-4) are a monovalent group selected from substituted or unsubstituted phenoxazinyl, substituted or unsubstituted phenothiazinyl, substituted or unsubstituted acridinyl, substituted or unsubstituted phenazinyl, and carbazolyl having, as a substituent, a disubstituted amino group substituted with an aromatic hydrocarbon group or a condensed polycyclic aromatic group.

9. The compound having a triphenylene ring structure according to claim 1, wherein Y in the general formula (1), (1-1), (1-2), (1-3), or (1-4) is a hydrogen atom or a deuterium atom.

10. A light-emitting material comprising the compound having a triphenylene ring structure of claim 1.

11. The light-emitting material according to claim 10, wherein the light-emitting material emits delayed fluorescence.

12. An organic electroluminescent device comprising a pair of electrodes, and one or more organic layers sandwiched between the pair of electrodes, wherein the compound having a triphenylene ring structure of claim 1 is used as a constituent material of at least one organic layer.

13. The organic electroluminescent device according to claim 12, wherein the organic layer produced with the compound having a triphenylene ring structure is a light emitting layer.

14. The organic electroluminescent device according to claim 12, wherein the organic layer produced with the compound having a triphenylene ring structure emits delayed fluorescence.

15. The organic electroluminescent device according to claim 12, wherein the organic layer produced with the compound having a triphenylene ring structure is an electron transport layer.

16. The organic electroluminescent device according to claim 12, wherein the organic layer produced with the compound having a triphenylene ring structure is a hole blocking layer.

17. The compound having a triphenylene ring structure according to claim 2, wherein X in the general formula (1), (1-1), (1-2), (1-3), or (1-4) is a monovalent group selected from substituted or unsubstituted phenoxazinyl, substituted or unsubstituted phenothiazinyl, substituted or unsubstituted acridinyl, substituted or unsubstituted phenazinyl, and carbazolyl having, as a substituent, a disubstituted amino group substituted with an aromatic hydrocarbon group or a condensed polycyclic aromatic group.

18. The compound having a triphenylene ring structure according to claim 2, wherein Y in the general formula (1), (1-1), (1-2), (1-3), or (1-4) is a monovalent group selected from substituted or unsubstituted phenoxazinyl, substituted or unsubstituted phenothiazinyl, substituted or unsubstituted acridinyl, substituted or unsubstituted phenazinyl, and carbazolyl having, as a substituent, a disubstituted amino group substituted with an aromatic hydrocarbon group or a condensed polycyclic aromatic group.

19. The compound having a triphenylene ring structure according to claim 2, wherein X and Y in the general formula (1), (1-1), (1-2), (1-3), or (1-4) are a monovalent group selected from substituted or unsubstituted phenoxazinyl, substituted or unsubstituted phenothiazinyl, substituted or unsubstituted acridinyl, substituted or unsubstituted phenazinyl, and carbazolyl having, as a substituent, a disubstituted amino group substituted with an aromatic hydrocarbon group or a condensed polycyclic aromatic group.

20. The compound having a triphenylene ring structure according to claim 2, wherein Y in the general formula (1), (1-1), (1-2), (1-3), or (1-4) is a hydrogen atom or a deuterium atom.
